(19) United States
(12) Patent Application Publication
(10) Pub. No.: US 2012/0064142 A1
(43) Pub. Date: Mar. 15, 2012

(54) POLYMERIC PHARMACEUTICAL DOSAGE FORM IN SUSTAINED RELEASE

(75) Inventors: Viness Pillay, Johannesburg (ZA); Yahya Essop Choonara, Johannesburg (ZA); Bongani Sibeko, Johannesburg (ZA); Sheri-Lee Harilall, Johannesburg (ZA); Samatha Pillay, Johannesburg (ZA); Girish Modi, Johannesburg (ZA); Sunny Esayegbemu Iyuke, Johannesburg (ZA); Dinesh Naidoo, Johannesburg (ZA)

(73) Assignee: UNIVERSITY OF THE WITWATERSRAND, JOHANNESBURG, Johannesburg (ZA)

(21) Appl. No.: 13/131,820

(22) PCT Filed: Nov. 30, 2009
(86) PCT No.: PCT/IB2009/007598

§ 371 Date: Nov. 14, 2011

(30) Foreign Application Priority Data
Nov. 30, 2008 (ZA) 2008/05625
Nov. 30, 2008 (ZA) 2008/05626

(51) Int. Cl. A61K 9/00; A61K 31/137; A61P 25/16; A61P 35/00; A61P 25/28; A61K 9/14; A61P 25/00; B82Y 5/00

(52) U.S. Cl. 424/423; 424/486; 514/654; 424/488; 424/487; 424/484; 424/430; 424/434; 424/422; 977/773; 977/906

(57) ABSTRACT

This invention relates to a polymeric pharmaceutical dosage form for the delivery, in use, of at least one pharmaceutical composition in a rate-modulated and site-specific manner. The dosage form comprises a biodegradable, polymeric, scaffold incorporating loaded with at least one active pharmaceutical ingredient (API). The polymer or polymers making up the scaffold degrade in a human or animal body in response to or in the absence of specific biological stimuli and, on degradation, release the API or APIs in an area where said stimuli are encountered. Preferably the polymeric scaffold is formed from poly (D1L-lactide) (PLA) and polymethacrylate (Eudragit S100/ES100) polymers.

### FIELD OF THE INVENTION

This invention relates to a polymeric pharmaceutical dosage form for the delivery of pharmaceutical compositions in a rate-modulated site-specific manner for oral administration or for targeted drug delivery as an implantable embodiment in a human or animal body. The invention extends to a method of manufacturing the polymeric pharmaceutical dosage form and to medicaments consisting of the polymeric pharmaceutical dosage form and at least one active pharmaceutical ingredient.

### BACKGROUND TO THE INVENTION

A site-specific micro- or nano-enabled polymeric configuration would, it is envisaged, serve to enhance the management of debilitating central nervous system disorders such as neurodegenerative disorders (e.g. Parkinson's disease, AIDS Dementia Complex (ADC) and brain cancers (e.g. Primary Central Nervous System Lymphoma (PCNSL).

Cognitive and mental impairments associated with ADC is effectively managed with zidovudine (AZT) therapy, however, bioavailability of the drug is limited due to first pass metabolism. Nanotechnology enables controlled and targeted drug release over predetermined periods, for nanosystems can be manipulated to react in a bioresponsive manner. Poorly soluble drugs can be incorporated into nanosystems for transportation into the Central Nervous System (CNS), due the ability of nanosystems to open tight junctions in the Blood Brain Barrier (BBB), indicating the applicability of this system for a multitude of drug to manage various conditions. The use of biodegradable, biocompatible polymers such as polycaprolactone and epsilon-caprolactone to synthesise a polymer scaffold into which the nanoparticles are dispersed serves to further extend drug release over several months, as the slow degradation of the scaffold allows for prolonged, controlled release of drug-loaded nanoparticles, negating the need for daily oral intake of medication to manage ADC, thereby enhancing the patients quality of life and also compliance with a treatment regime.

Polymeric nanotechnology has been extensively researched for its application in cancer therapy [13]. Cancer and neurodegenerative disease treatment are similar in that they both require targeted drug delivery to optimize bioavailability and reduce systemic side effects experienced with CNS drugs. Nano-enabled polymeric drug delivery devices have the potential to (i) maintain therapeutic levels of drug, (ii) reduce harmful side effects, (iii) decrease the quantity of drug needed, (iv) reduce the number of dosages (dosage frequency), and (v) facilitate the delivery of drugs with short in vivo half-lives (Kohane, 2006; Gelperina et al., 2005; Langer, 1998).

Further background to this invention involves the use of a site-specific micro- or nano-enabled polymeric pharmaceutical dosage form in conditions/diseases such as Neurodegenerative disorders. Parkinson's disease (PD) (one example of such a disease) is one of the most common and severely debilitating neurodegenerating diseases [2]. This motor condition is characterized by a progressive loss of dopamine-producing neurons in the substantia nigra of the brain. The fundamental symptoms consist of rigidity, bradykinesia, distinctive tremor and postural instability (Nyholm, 2007).

Currently, the main therapy for the treatment of PD is levodopa however, with chronic use comes a host of limitations. L-dopa is essentially the levorotatory isomer of dihydroxy-phenylalanine (dopa) which is the metabolic precursor of dopamine. L-dopa presumably is converted into dopamine in the basal ganglia. The reason for the formulation and current widespread use of the levorotatory isomer (L-dopa) is to enhance transport of the drug across the BBB. Initial therapy with L-dopa significantly restores normal functioning for the patient with PD and every PD-patient will need this treatment at some time during the course of the disease (Samii et al., 2007). However the major limitation to the use of L-dopa comes after long term use of the oral dosage form. The phenomenon which arises is known as the ‘end-of-dose wearing-off’, where the therapeutic benefits of each dose of L-dopa lasts for shorter periods [7]. The patient begins to experience motor fluctuations prior to the time of the next dose; this is when the prescribed dose is no longer able to effectively manage the symptoms of the disease. In many patients, ‘off’ periods of motor immobility are associated with pain, panic attacks, severe depression, confusion and a sense of death [8], which makes the clinical status even more distressing for patients. Clinicians will attempt to overcome this phenomenon by either increasing the frequency/amount of the dose or by replacing the immediate release preparations with a sustained release preparation (Sinemet® CR). Increase of the dose puts the patient at risk for dyskinesia (the inability to control muscles) which occurs at peak plasma drug levels [10]. The dose will also need to be increased on a regular basis as to overcome “the wearing off” effect. With an increase in dose comes an increase in side-effects. Sinemet® CR does provide benefit in that it is able to maintain drug plasma levels however this is only for a 24-hour period [9]. Side-effects such as dizziness, insomnia, abdominal pain, dyskinesia, headache and depression are still experienced with the sustained release preparation. The inclusion of carbidopa (75-100 mg required daily) tends to exacerbate psychiatric, gastrointestinal and motor side-effects. Patients also find that while the dosing schedule proves convenient, there is still evidence of dyskinesia (Pahwa et al., 1996). There have also been reports that, with both the Sinemet® preparations, food retards absorption of the drug [11].

A drug delivery device implanted into the subarachnoid cavity of the brain does not require transport across the BBB and so makes the need for the L-isomer (1-dopa) or carbidopa redundant in this drug delivery device. In the present invention it is preferable to load dopamine hydrochloride into the device so as to avoid the need for metabolism to the active and peripheral loss of the drug thereby increasing its bioavailability. The inclusion of nanoparticles in a polymeric scaffold is advantageous for targeted drug delivery as the nanoparticles allow for higher drug loading, due to its high surface area to volume ratio in comparison to other polymeric systems, and are able to facilitate opening of tight junctions between cells for penetrating the BBB (but do not need to penetrate BBB).

Furthermore, by employing biodegradable polymers during formulation one obviates the need for surgical procedures to remove the drug delivery device once its drug-load has been depleted [14]. The employment of statistical design in the optimization of drug delivery system (DDS) allows for effective and efficient research and design processes. The Box-Behnken design examines the relationship between one or more response variables and a set of quantitative experimental parameters. It is a quadratic design that does not contain an embedded factorial or fractional factorial design. This design requires 3 levels of each factor (Patel, 2005). The design was selected to evaluate the influence the process variables have on such parameters such as in vitro drug release and degradation of barium-alginate scaffolds and CAP DA-loaded nanoparticles for intracranial implantation for the treatment of PD.

In yet further background to the invention, it is envisaged that novel pharmaceutical drug delivery systems based on biocompatible and biodegradable polymers such as polylactic acid (PLA), polylactic-co-glycolic acid (PLGA) and polyvinyl alcohol (PVA) provide solutions to therapeutic challenges associated with conventional drug delivery systems.

The majority of these polymers possess unique inherent physicochemical and physicomechanical properties that facilitate the tailoring of drug delivery systems for a specific therapeutic need. The availability of numerous polymer fabrication techniques reported enables researchers to manipulate the physicochemical and physicomechanical properties of the material in order to obtain optimum drug release kinetics from innovative delivery systems. Phase separation processes have been employed for the development of polymeric membranes for various applications.

Typical examples of polymeric membranes include applications in microfiltration, ultra-filtration, reverse osmosis and gas separation. A huge variety of polymer architectures and functions can be gained by phase separation and hence membrane technology can be extended to biomedical and pharmaceutical applications for example wound healing, tissue engineering and drug delivery. The combination of technologies such as micro- or nanotechnology and membrane technology can lead to the realization of advanced drug delivery systems. This combination of technologies may translate into systems capable of multiple bioactive loading where a bioactive compound is entrapped within the nanostructures embedded in the polymeric membranous scaffold loaded with a different bioactive compound for treatment of various illnesses, for example, in primary brain tumors, or systems for extended drug release where the membrane increases the diffusion path length of the drug from the embedded micro- or nanostructures.

Nanotechnology, a conventional and prospective field in drug delivery research has resulted in the development of efficient nanoscale drug delivery systems for various therapeutic applications. Compared to other polymer based drug delivery devices, nanoparticles (NPs) drug vehiculant systems offer unique advantages owing to their nanoscale dimensions in the range of 10 to 1000 nm. These minute powerful systems have the ability to release an encapsulated drug in a controlled manner and posses the ability to penetrate cellular structures of tissues/organs when tailor made for active targeting.

The release of chemotherapeutic agents from implantable drug-polymer carrier systems intended for local delivery can further be delayed and modulated by embedding drug loaded nanoparticles within a polymer matrix in the place of pure drug. The composite system will result in an increase drug diffusion path length drug release will be delayed. In addition, the burst effect observed with many nanoparticle formulations will be eliminated. The combined unique hydration and swelling dynamics of each system gives rise to higher order drug release kinetics and drug modulation effect compared to a matrix system loaded with pure drug rendering the composite system more suitable for long term drug delivery.

### OBJECT OF THE INVENTION

It is an object of this invention to provide a polymeric pharmaceutical dosage form for the delivery of pharmaceutical compositions in a rate-modulated site-specific manner to the human or animal body and, more particularly, to a polymeric configuration that is a micro- or nano-enabled scaffold capable of controlled, site-specific delivery of at least one active pharmaceutical composition. The invention also provides for a method of manufacturing the said polymeric pharmaceutical dosage form.

### SUMMARY OF THE INVENTION

In accordance with this invention there is provided a polymeric pharmaceutical dosage form for the delivery, in use, of at least one pharmaceutical composition in a rate-modulated and site-specific manner, said dosage form comprising a biodegradable, polymeric, scaffold incorporating nanoparticles, alternatively microparticles loaded with at least one active pharmaceutical ingredient (API) which, in use, are released from said scaffold as the polymer or polymers degrade in a human or animal body, the polymer or polymers being selected to degrade in response to or in the absence of specific biological stimuli and, thus, release the API or APIs in an area where said stimuli are encountered.

There is also provided for the polymeric scaffold to be a membranous-like polymeric scaffold which, in use, releases the or each API in a desired rate modulated manner which is achievable by selecting one or more polymers making up the scaffold according to the rate of biological degradation of said polymers and the consequent release of the or each API in the human or animal body.

There is further provided for the or each polymer making up the polymeric scaffold to be hydrophilic, preferably polyvinyl alcohol (PVA), alternatively hydrophobic, preferably polylactic acid (PLA), further alternatively a combination of hydrophilic and hydrophobic polymers, preferably selected from the group consisting of polycaprolactone (PCL), pectins, and alginates as native polymers. Preferably the polymeric scaffold is formed from poly (D,L-lactide) (PLA) and polymethacrylate (Eudragit S100/ES100) polymers.

There is further provided for at least one the or each polymer making up the polymeric scaffold to be include a modifier chemical which, in use, causes the or each polymer to undergo, in use, a controlled swelling, shrinking and/or erosion, for the modifier to be selected from a group of substances that interact with the or each polymer, one example being HCl which reacts with alginate to reduce the swellibility of the latter.

There is also provided for the inherent polymeric structure of the native polymer or polymers to be manipulated through crosslinking using crosslinking reagents, preferably with biocompatible inorganic salts which may be ionic of a mono-, di-, or trivalent nature, preferably selected from the group consisting sodium chloride, aluminium chloride or calcium chloride.

There is further provided for a desired release rate-modulatable polymeric configuration to be attained in use by a combination of any one of a number of combination permutations of hydrophilic and hydrophobic polymeric, preferably PCL, matrices, active pharmaceutical compositions and inorganic salt(s), and wherein the release profile of the pharmaceutical composition or compositions are governed by the crosslinking reagent, polymer matrix size and hydration, porosity, embedded nanostructures and the architectural structure of the resulting polymeric network as well as the degree of hydration of the polymer or polymers which, in turn, depends on the pKa, concentration and valence of release rate-modulating chemical substances used.

There is further provided for the API or APIs to display, in use, flexible yet-rate modulated release kinetics, thereby providing a steady supply of pharmaceutical compositions over the desired period of time that may vary from hours to months depending on the polymeric configuration.

There is further provided for the dosage form to be orally ingestible in use and for it to be in the form of a tablet, caplet or capsule. Alternatively there is provided for the dosage form to be surgically implantable in use. Further alternatively there is provided for the dosage form to be insertable, in use, into a body cavity such as a nasal passage, rectum or vagina.

The invention also provides for the dosage form to be adapted to treat, in use, a chronic medical condition, preferably Parkinson's disease, and for the dosage form to comprise a barium-alginate scaffold incorporating CAP dopamine-loaded nanoparticles.

The invention also provides for the dosage form to be adapted to treat, in use, a chronic medical condition, preferably brain tumors, and for the dosage form to comprise a membranous-like polymeric scaffold incorporating API-loaded nanoparticles.

The invention also provides for the dosage form to be adapted to treat, in use, a chronic medical condition, preferably Aids Dementia Complex, and for the dosage form to comprise a polymeric scaffold incorporating API-loaded nanoparticles.

The invention extends to a method of preparing a polymeric pharmaceutical dosage form for the delivery, in use, of at least one pharmaceutical composition in a rate-modulated and site-specific manner, said method comprising preparing a biodegradable, polymeric, scaffold, loading nanoparticles, alternatively microparticles with at least one active pharmaceutical ingredient (API) and incorporating the nanoparticles, alternatively microparticles into the scaffold so that the nanoparticles, alternatively microparticles, and, consequently, the API is released, in use, from said scaffold as the polymer or polymers degrade in a human or animal body, the polymer or polymers being selected to degrade in response to or in the absence of specific biological stimuli and, thus, release the API or APIs in an area where said stimuli are encountered.

There is also provided for the polymeric scaffold to be a membranous-like polymeric scaffold which, in use, releases the or each API in a desired rate modulated manner which is achievable by selecting one or more polymers making up the scaffold according to the rate of biological degradation of said polymers and the consequent release of the or each API in the human or animal body.

There is further provided for the or each polymer making up the polymeric scaffold to be hydrophilic, preferably polyvinyl alcohol (PVA), alternatively hydrophobic, preferably polylactic acid (PLA), further alternatively a combination of hydrophilic and hydrophobic polymers, preferably selected from the group consisting of polycaprolactone (PCL), pectins, and alginates as native polymers. Preferably the polymeric scaffold is formed from poly (D,L-lactide) (PLA) and polymethacrylate (Eudragit S100/ES100) polymers. There is also provided for the inherent polymeric structure of the native polymer or polymers to be manipulated through crosslinking using crosslinking reagents, preferably with biocompatible inorganic salts which may be ionic of a mono-, di-, or trivalent nature, preferably selected from the group consisting sodium chloride, aluminium chloride or calcium chloride.

There is further provided for a desired release rate-modulatable polymeric configuration to be attained in use by a combination of any one of a number of combination permutations of hydrophilic and hydrophobic polymeric, preferably PCL, matrices, active pharmaceutical compositions and inorganic salt(s), and wherein the release profile of the pharmaceutical composition or compositions are governed by the crosslinking reagent, polymer matrix size and hydration, porosity, embedded nanostructures and the architectural structure of the resulting polymeric network as well as the degree of hydration of the polymer or polymers which, in turn, depends on the pKa, concentration and valence of release rate-modulating chemical substances used.

There is further provided for the API or APIs to display, in use, flexible yet rater modulated release kinetics, thereby providing a steady supply of pharmaceutical compositions over the desired period of time that may vary from hours to months or years depending on the polymeric configuration.

There is further provided for the dosage form to be orally ingestible in use and for it to be in the form of a tablet, caplet or capsule. Alternatively there is provided for the dosage form to be surgically implantable in use. Further alternatively there is provided for the dosage form to be insertable, in use, into a body cavity such as a nasal passage, rectum or vagina or after a surgical procedure.

According to the invention, there is provided a method of obtaining rate-modulated drug release characteristics from an implantable polymeric, nano-enabled pharmaceutical dosage form and a biodegradable drug delivery system.

Further, according to the invention, polymeric permutations have been employed in simulating a polymer configuration to deliver drug-loaded polymeric nanostructures, preferably nanoparticles, with superior drug permeability to attain selected drug release profiles. The implantable polymeric configuration, comprising biodegradable polymers and drug-loaded nanostructures may be employed for achieving rate-modulated drug release in a site-specific manner to various organs in a human or animal body.

There is provided for the said nanostructures to facilitate in achieving selected release profiles in order to improve the delivery of bioactives to an intended site of action.

Further, there is provided for the polymeric material employed in formulating the said polymeric configuration and pharmaceutical dosage form to be a hydrophilic, hydrophobic or a combination of the two polymeric types. Preferably such polymers are from the group comprising biodegradable polymers such as polycaprolactone (PCL), pectins, and alginates.

There is also provided for the pharmaceutical dosage form to be prepared with the said polymers by manipulation of the inherent polymeric structure of the native polymer(s) through crosslinking using crosslinking reagents.

The crosslinking reagents are selected from a class of biocompatible inorganic salts, used in the crosslinking reactions of the polymer or polymer and pharmaceutical agent, and are ionic of either mono-, di-, or trivalent nature, examples of which are sodium chloride, aluminium chloride or calcium chloride.

There is also provided for the attainment of a release rate-modulatable polymeric configuration composed of permutations of a hydrophilic and hydrophobic polymeric matrix, preferably PCL, active pharmaceutical compositions, inorganic salt(s), wherein the release profile of the pharmaceutical composition(s) is governed by the crosslinking reagent, polymer matrix size and hydration, porosity, embedded nanostructures and the architectural structure of the resulting polymeric network.

There is provided for the release profiles to display flexible yet-rate modulated release kinetics, thereby providing a steady supply of pharmaceutical compositions over the desired period of time that may vary from hours to months.

There is also provided for a polymeric nano-enabled scaffold to be employed for the treatment of chronic conditions, like Parkinson's disease, where there is no sign of a cure or effective treatment

There is also provided for the pharmaceutical dosage form to be prepared preferably from a barium-alginate scaffold and incorporating CAP dopamine-loaded nanoparticles.

Further, there is provided a method of manufacturing the polymeric configuration, the biodegradable pharmaceutical dosage form and the nanostructures containing active pharmaceutical compositions that may or may not be embedded within the said polymeric configuration, substantially as described herein.

Further, there is also provided a method of obtaining rate-modulated drug release characteristics from a membranous polymeric scaffold and a biodegradable pharmaceutical dosage form formulated from the said scaffold comprising active pharmaceutical compositions that may or may not be embedded within micro- or nanostructures.

There is also provided for the said micro- or nanostructures to achieve selected release profiles in order to improve the delivery of bioactives to an intended site of action.

Further, there is provided for the said membranous polymeric scaffold to achieve selected release profiles in order to improve the delivery of bioactives to an intended site of action due to the physicochemical and physicomechanical properties of the said scaffold.

There is also provided for the polymeric material employed in formulating the said membranous scaffold and pharmaceutical dosage form to be a hydrophilic, hydrophobic or a combination of the two polymeric types. Preferably, such polymers may be from the group comprising polyvinyl alcohol (PVA) (hydrophilic) or polylactic acid (PLA) (hydrophobic) and their variants or various permutations of polymer-types. The scaffold is prepared with the said polymers by manipulation of the inherent polymeric structure of the native polymer(s) through phase-separation and crosslinking using crosslinking reagents.

There is also provided for the said scaffold to be prepared with the said polymers by manipulation of the inherent polymeric structure of the native polymer(s) through phase-separation and addition of chemical substances from among the group comprising, preferably triethanolamine to function as nodal points on the polymeric backbone structure for the conjugation of bioactive molecules.

There is also provided for the crosslinking reagents to be selected from a class of biocompatible organic or inorganic salts, used in the crosslinking reactions of the polymer or polymer and pharmaceutical agent, and are ionic of either mono-, di-, or trivalent nature, examples of which are sodium chloride, aluminium chloride or calcium chloride.

There is also provided for a release rate-modulatable membranous polymeric scaffold composed of permutations of a hydrophilic and hydrophobic polymeric matrix, preferably PVA and PLA, a pharmaceutical agent, inorganic salt(s), chemical substances, such as triethynolamine, wherein the release profile of the pharmaceutical agent from the system is governed by the crosslinking reagent, membrane pore size, embedded nanostructures and the architectural structure of the resulting polymeric network.

Further, according to the invention, the pre-determined rate-modulated release profile is controlled by the rate of polymeric hydration within the system which depends on the pKa, concentration and valence of the release rate-modulating chemical substances used.

Further, according to the invention, the pre-determined rate-modulated release profile is controlled by the rate of diffusion of the embedded micro- or nanostructures that may also influence polymeric hydration within the system which depends on the pKa, concentration and valence of the release rate-modulating chemical substances used.

There is also provided for the release profiles to display flexible rate-modulated release kinetics, thereby providing a steady supply of a pharmaceutical agent over the desired period of time that may vary from hours to months.

According to another aspect of the invention, an oral drug delivery system is derived from the membranous polymeric scaffold consisting of the said membrane enclosed within a protective platform; in use, the said protective platform may be a capsule.

The drug delivery system prepared by phase separation of polymeric materials, as described above may be an oral or an implantable drug delivery system.

Further, according to the invention, there is provided a method of manufacturing the said membranous polymeric scaffold, the biodegradable pharmaceutical dosage form and the micro- or nanostructures containing active pharmaceutical compositions that may or may not be embedded within the said micro- or nanostructures, substantially as described herein.

There is also provided for a method of manufacturing the said micro- or nano structures, preferably from poly (D,L-lactide) (PLA) and polymethacrylate (Eudragit S100/ES100) polymers.

### DESCRIPTION OF EXAMPLES OF THE INVENTION

The above and additional features of the invention will be described below with reference to three non-limiting examples namely a biodegradable cellulose acetate phthalate nano-enabled scaffold device (NESD) for subarachnoid implantation for targeted dopamine delivery in Parkinson's disease (Example 1), a biodegradable polycaprolactone nano-enabled implantable scaffold (PNIS) for modulated site-specific drug release in the treatment of Aids Dementia Complex (Example 2) and a nano-enabled biopolymeric membranous scaffold (NBMS) for site-specific drug delivery in the treatment of primary central nervous system lymphoma (Example 3) and the following figures in which:

FIG. 1 is a schematic representation of the mechanism of drug delivery into the brain;

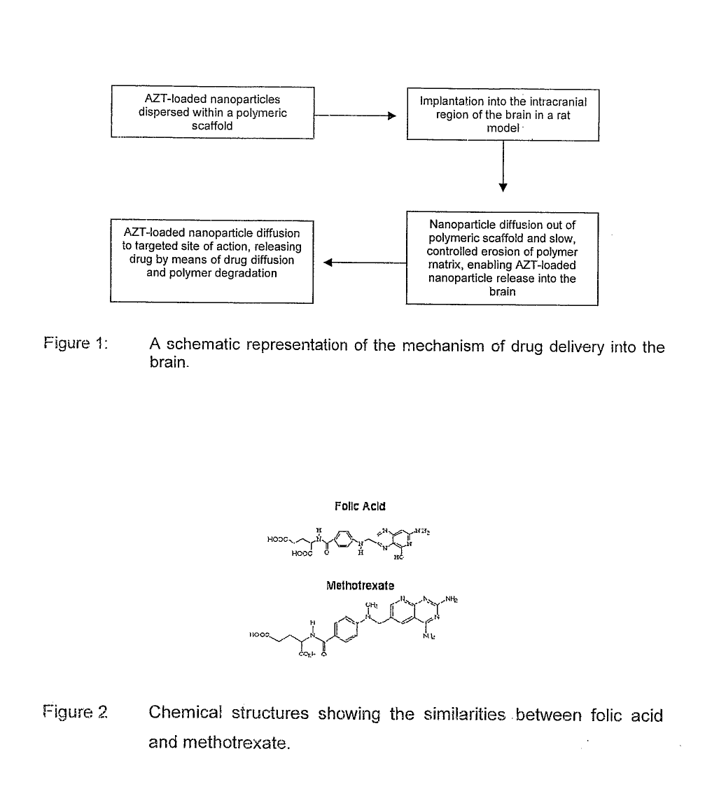

FIG. 2 illustrates chemical structures showing the similarities between folic acid and methotrexate;

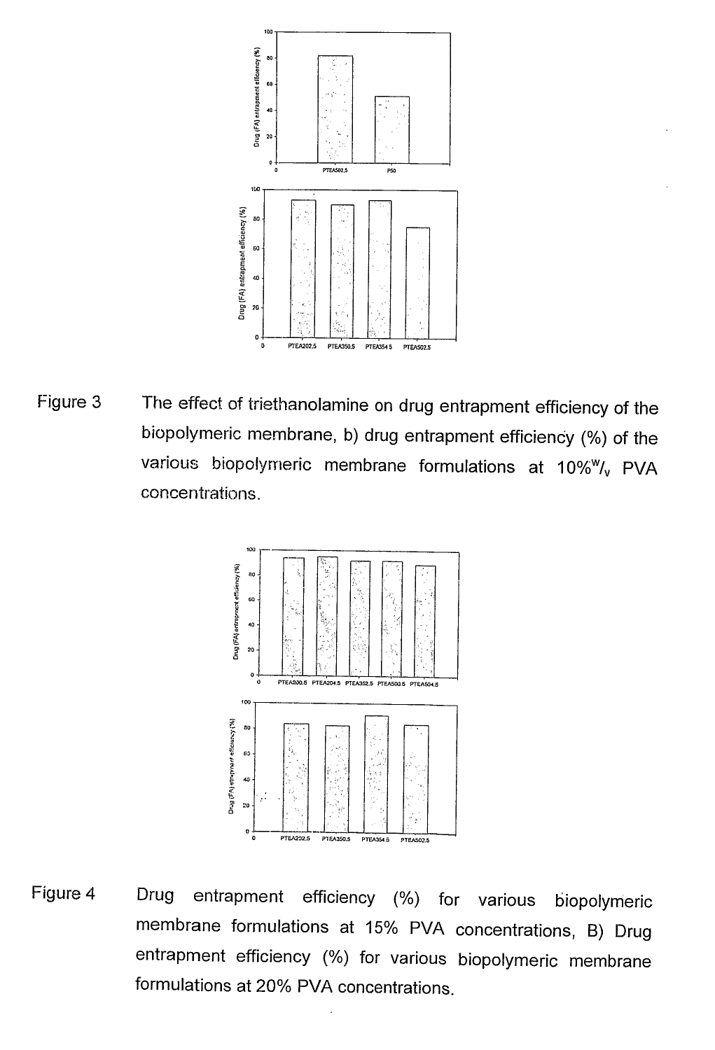

FIG. 3 shows the effect of triethanolamine on drug entrapment efficiency of the biopolymeric membrane, b) drug entrapment efficiency (%) of the various biopolymeric membrane formulations at 10% w/v PVA concentrations;

FIG. 4 shows drug entrapment efficiency (%) for various biopolymeric membrane formulations at 15% PVA concentrations, B) Drug entrapment efficiency (%) for various biopolymeric membrane formulations at 20% PVA concentrations;

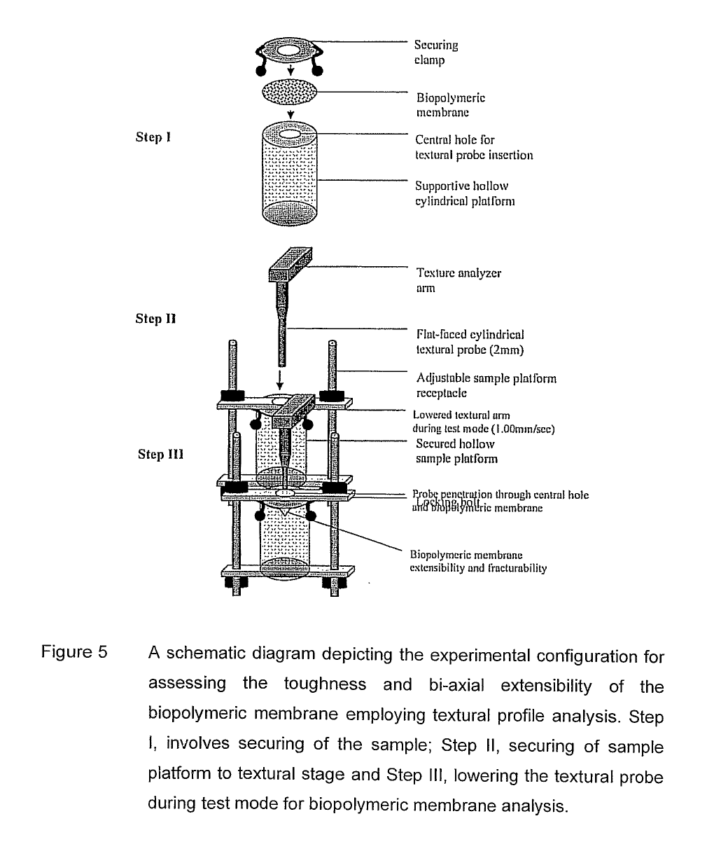

FIG. 5 is a schematic diagram depicting the experimental configuration for assessing the toughness and bi-axial extensibility of the biopolymeric membrane employing textural profile analysis. Step I, involves securing of the sample; Step II, securing of sample platform to textural stage and Step III, lowering the textural probe during test mode for biopolymeric membrane analysis;

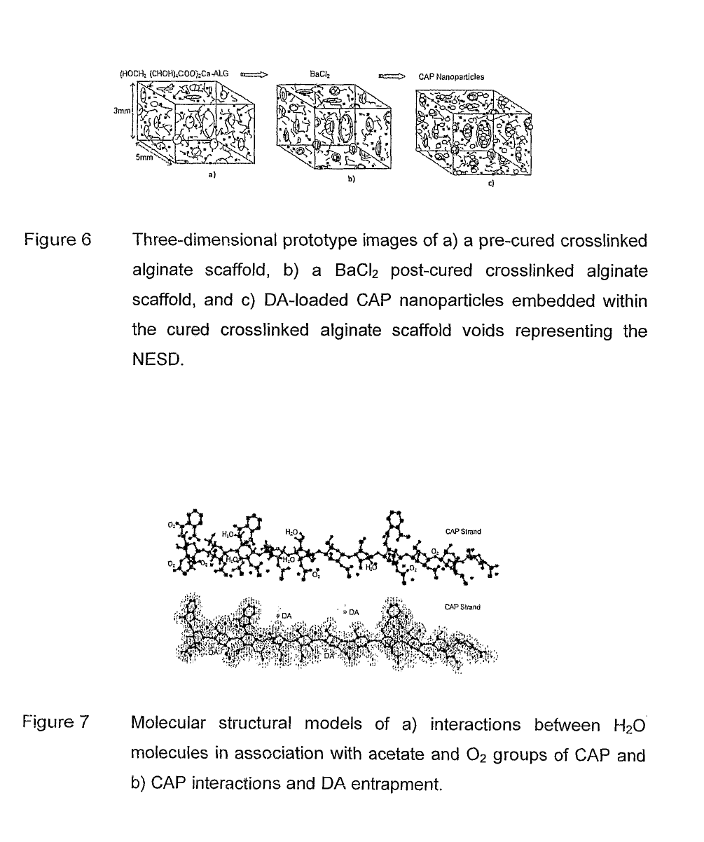

FIG. 6 shows three-dimensional prototype images of a) a pre-cured crosslinked alginate scaffold, b) a BaCl₂ post-cured crosslinked alginate scaffold, and c) DA-loaded CAP nanoparticles embedded within the cured crosslinked alginate scaffold voids representing the NESD;

FIG. 7 depicts molecular structural models of a) interactions between H₂O molecules in association with acetate and O₂ groups of CAP and b) CAP interactions and DA entrapment;

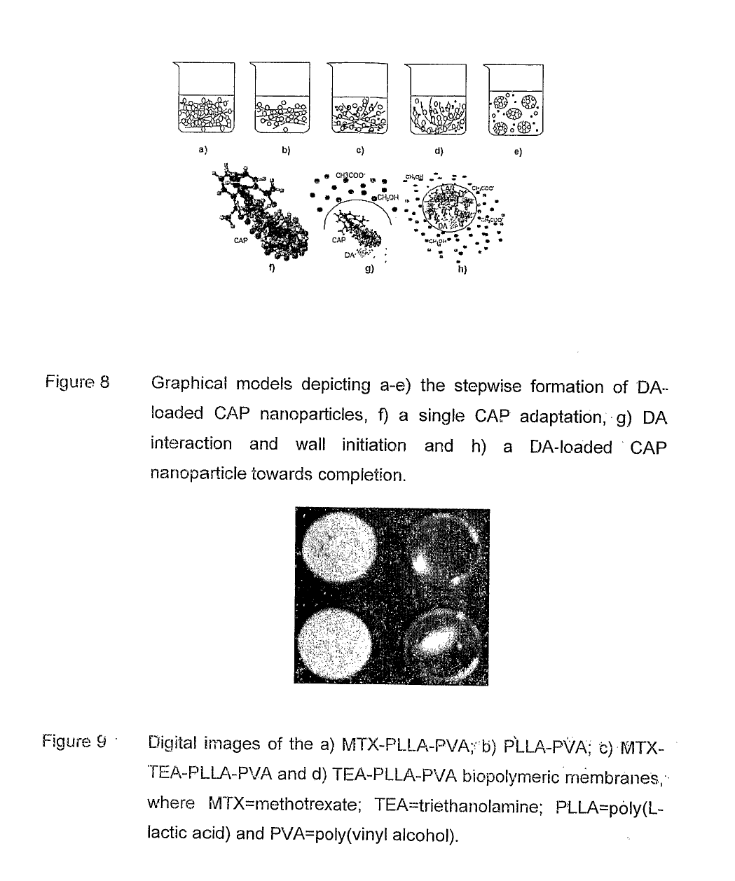

FIG. 8 presents graphical models depicting a-e) the stepwise formation of DA-loaded CAP nanoparticles, f) a single CAP adaptation, g) DA interaction and wall initiation and h) a DA-loaded CAP nanoparticle towards completion;

FIG. 9 are digital images of the a) MTX-PLLA-PVA; b) PLLA-PVA; c) MTX-TEA-PLLA-PVA and d) TEA-PLLA-PVA biopolymeric membranes, where MTX=methotrexate; TEA=triethanolamine; PLLA=poly(L-lactic acid) and PVA=poly(vinyl alcohol);

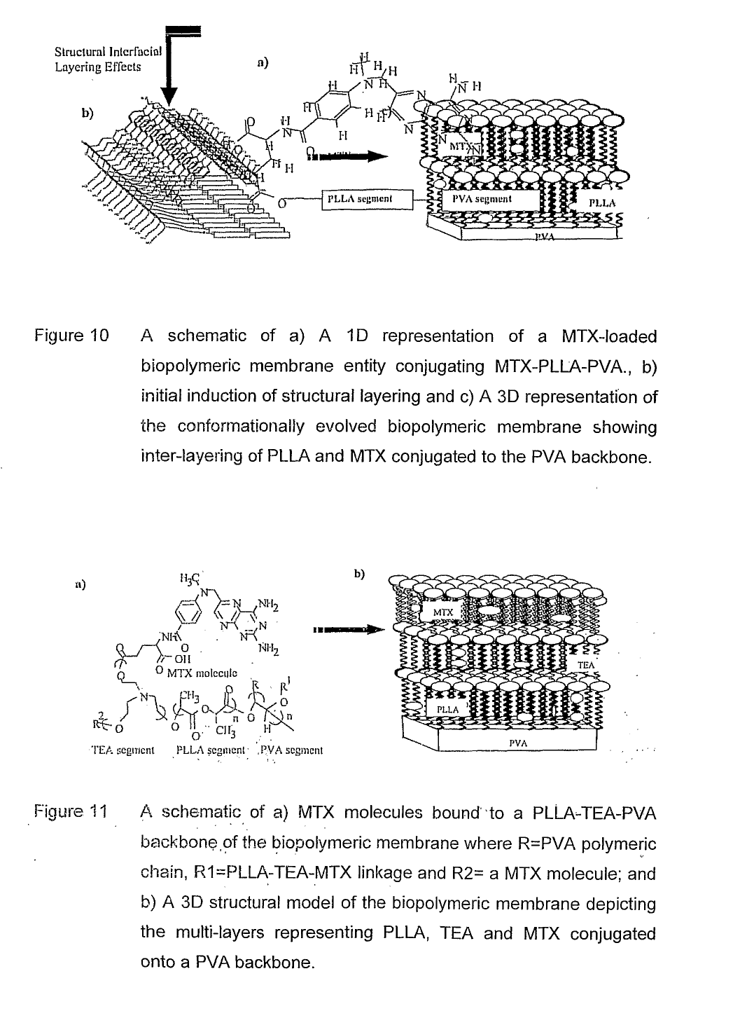

FIG. 10 is a schematic of a) A 1D representation of a MTX-loaded biopolymeric membrane entity conjugating MTX-PLLA-PVA, b) initial induction of structural layering and c) A 3D representation of the conformationally evolved biopolymeric membrane showing inter-layering of PLLA and MTX conjugated to the PVA backbone;

FIG. 11 is a schematic of a) MTX molecules bound to a PLLA-TEA-PVA backbone of the biopolymeric membrane where R═PVA polymeric chain, R1=PLLA-TEA-MTX linkage and R2=a MTX molecule; and b) A 3D structural model of the biopolymeric membrane depicting the multi-layers representing PLLA, TEA and MTX conjugated onto a PVA backbone;

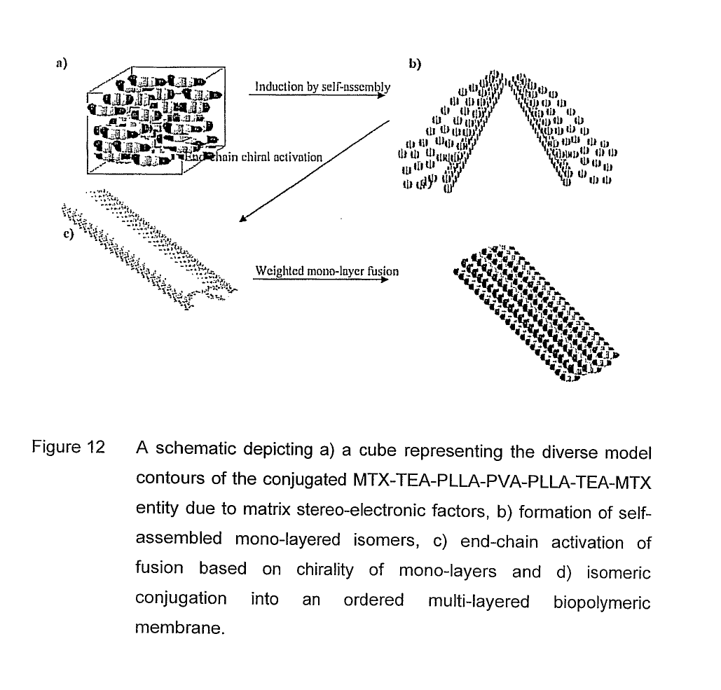

FIG. 12 is a schematic depicting a) a cube representing the diverse model contours of the conjugated MTX-TEA-PLLA-PVA-PLLA-TEA-MTX entity due to matrix stereo-electronic factors, b) formation of self-assembled mono-layered isomers, c) end-chain activation of fusion based on chirality of mono-layers and d) isomeric conjugation into an ordered multi-layered biopolymeric membrane;

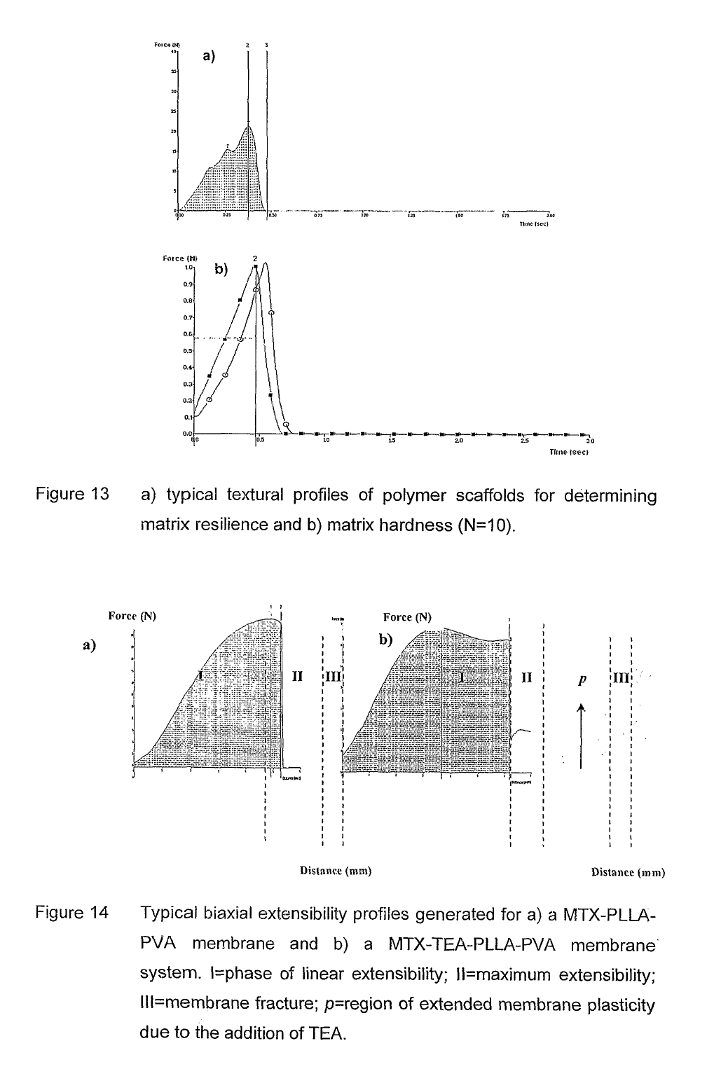

FIG. 13 illustrates a) typical textural profiles of polymer scaffolds for determining matrix resilience and b) matrix hardness (N=10);

FIG. 14 illustrates typical biaxial extensibility profiles generated for a) a MTX-PLLA-PVA membrane and b) a MTX-TEA-PLLA-PVA membrane system. I=phase of linear extensibility; II=maximum extensibility; III=membrane fracture; p=region of extended membrane plasticity due to the addition of TEA;

FIG. 15 SEM images (1 mm=0.5 μm) of a BaCl₂ a) un-cured and b) cured crosslinked alginate scaffold surface, c) DA-free nanoparticles, d) DA-loaded nanoparticles, and TEM images (1 mm=40 nm) of e) DA-free nanoparticles and f) DA-loaded nanoparticles;

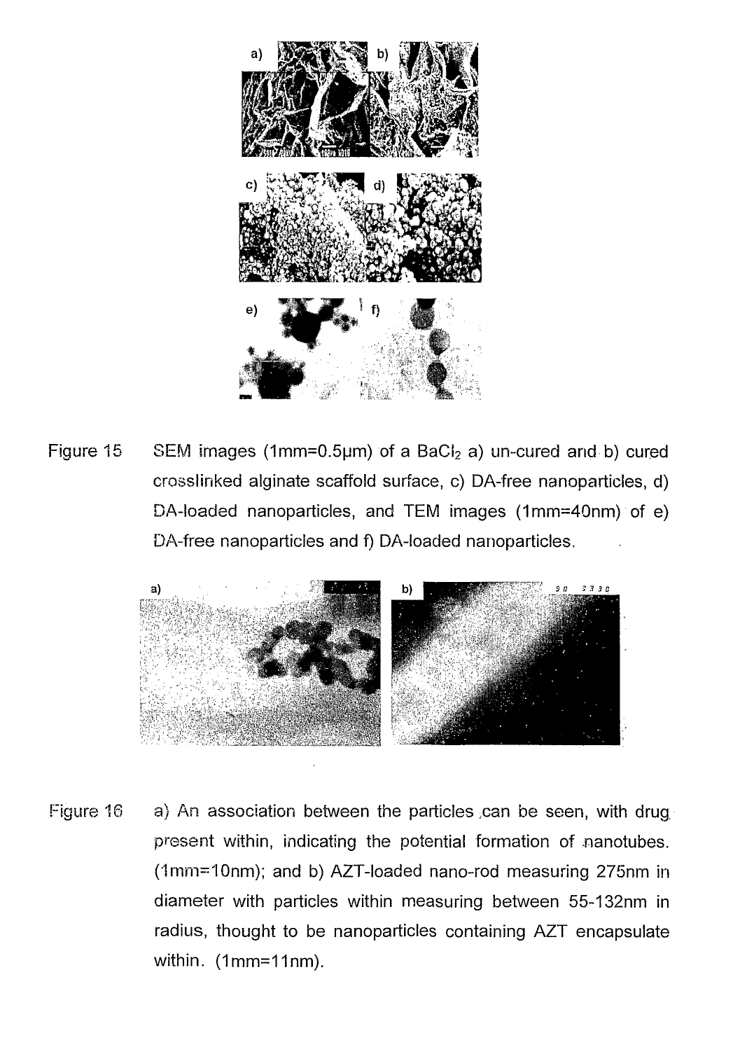

FIG. 16 illustrates a) An association between the particles can be seen, with drug present within, indicating the potential formation of nanotubes. (1 mm=10 nm); and b) AZT-loaded nano-rod measuring 275 nm in diameter with particles within measuring between 55-132 nm in radius, thought to be nanoparticles containing AZT encapsulate within. (1 mm=11 nm);

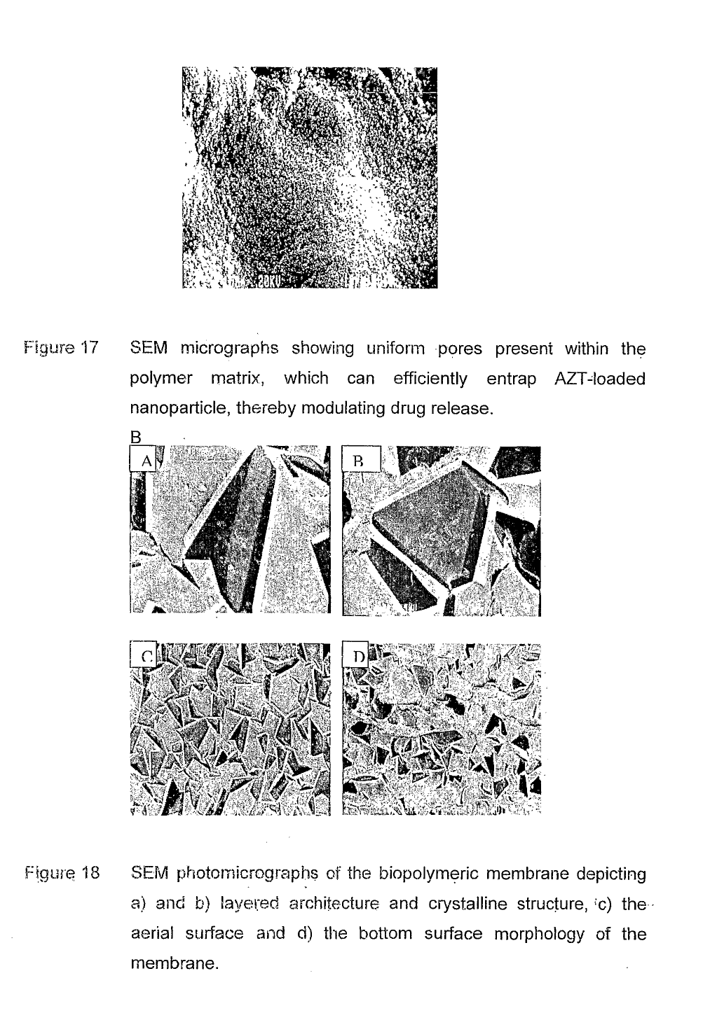

FIG. 17 SEM micrographs showing uniform pores present within the polymer matrix, which can efficiently entrap AZT-loaded nanoparticle, thereby modulating drug release;

FIG. 18 SEM photomicrographs of the biopolymeric membrane depicting a) and b) layered architecture and crystalline structure, c) the aerial surface and d) the bottom surface morphology of the membrane;

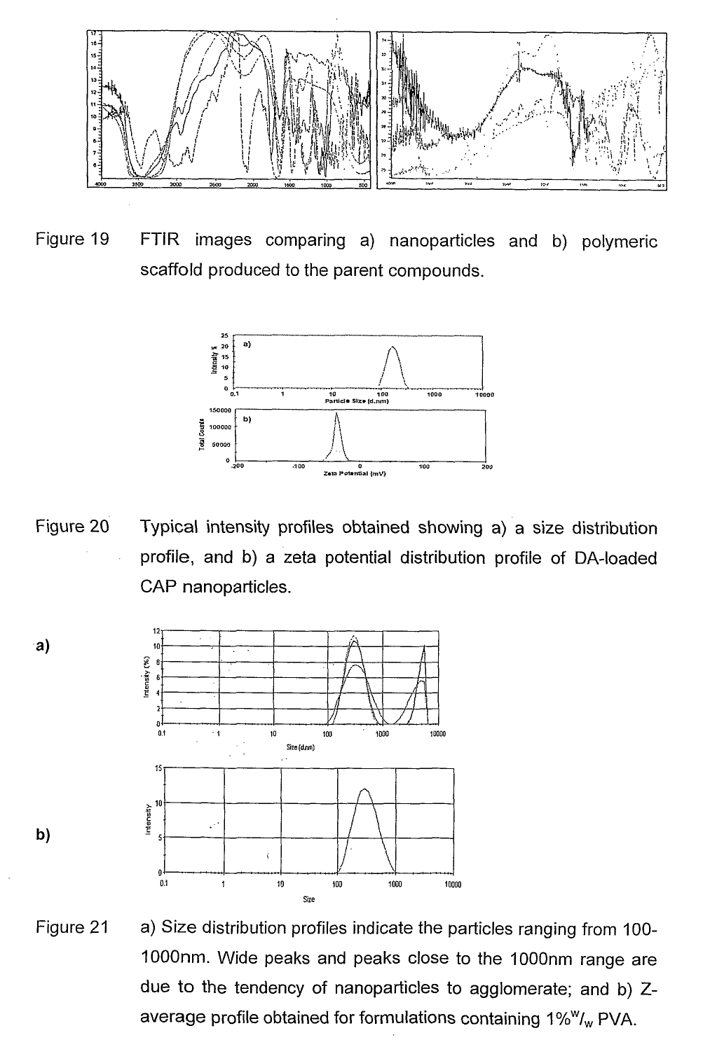

FIG. 19 FTIR images comparing a) nanoparticles and b) polymeric scaffold produced to the parent compounds;

FIG. 20 typical intensity profiles obtained showing a) a size distribution profile, and b) a zeta potential distribution profile of DA-loaded CAP nanoparticles;

FIG. 21 a) Size distribution profiles indicate the particles ranging from 100-1000 nm. Wide peaks and peaks close to the 1000 nm range are due to the tendency of nanoparticles to agglomerate; and b) Z-average profile obtained for formulations containing 1% w/w PVA;

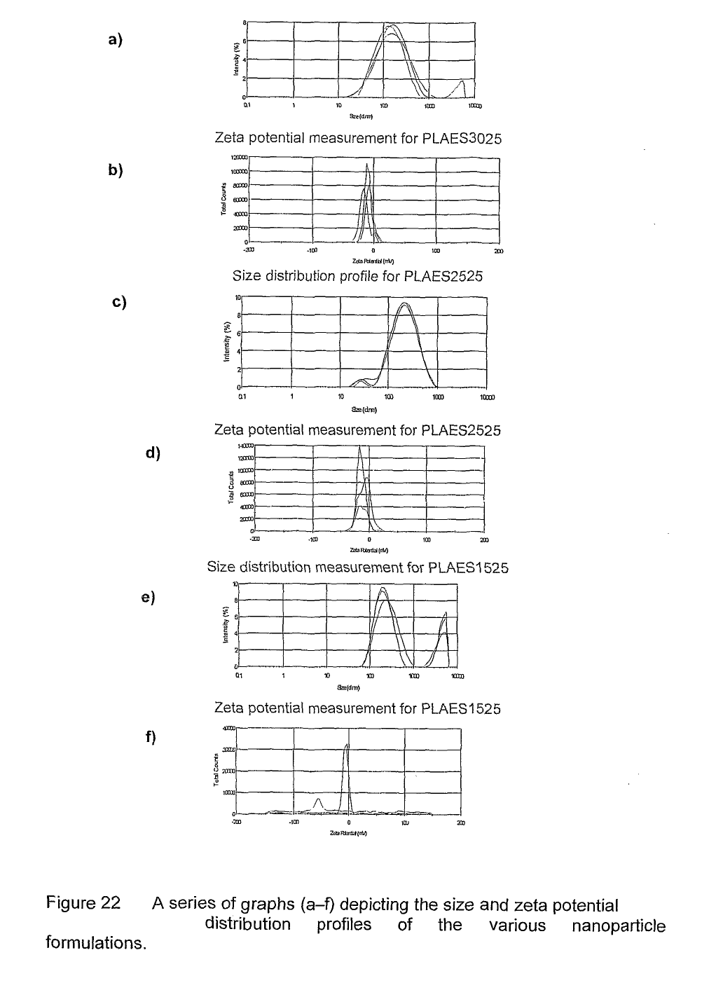

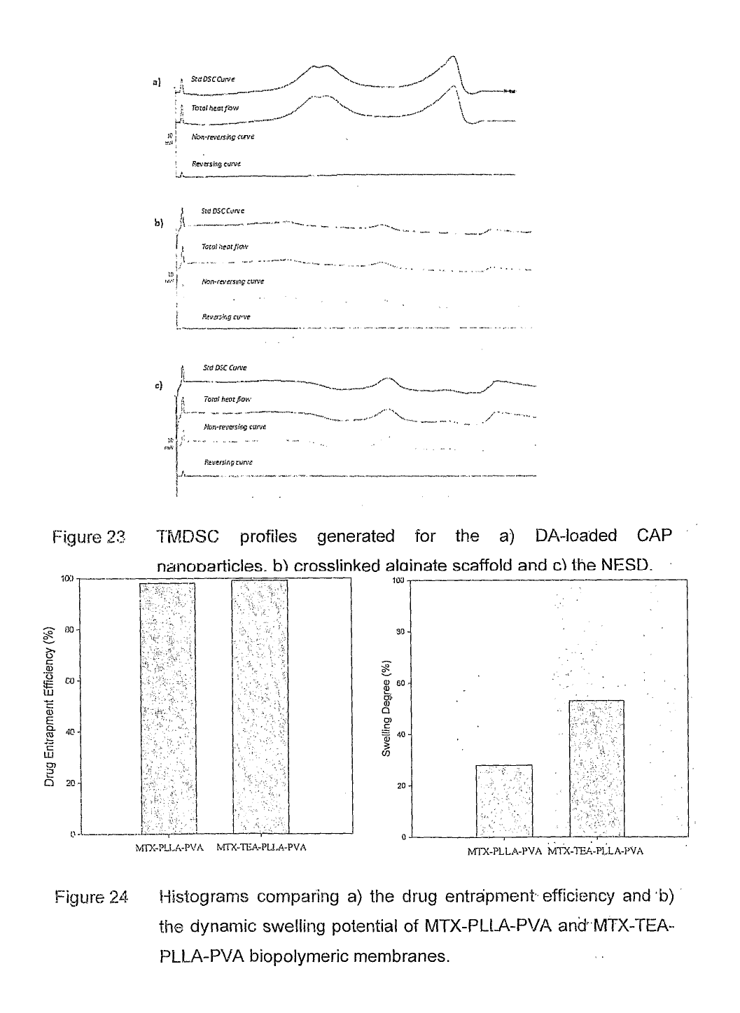

FIG. 22 a series of graphs (a-f) depicting the size and zeta potential distribution profiles of the various nanoparticle formulations;

FIG. 23 TMDSC profiles generated for the a) DA-loaded CAP nanoparticles, b) crosslinked alginate scaffold and c) the NESD;

FIG. 24 histograms comparing a) the drug entrapment efficiency and b) the dynamic swelling potential of MTX-PLLA-PVA and MTX-TEA-PLLA-PVA biopolymeric membranes;

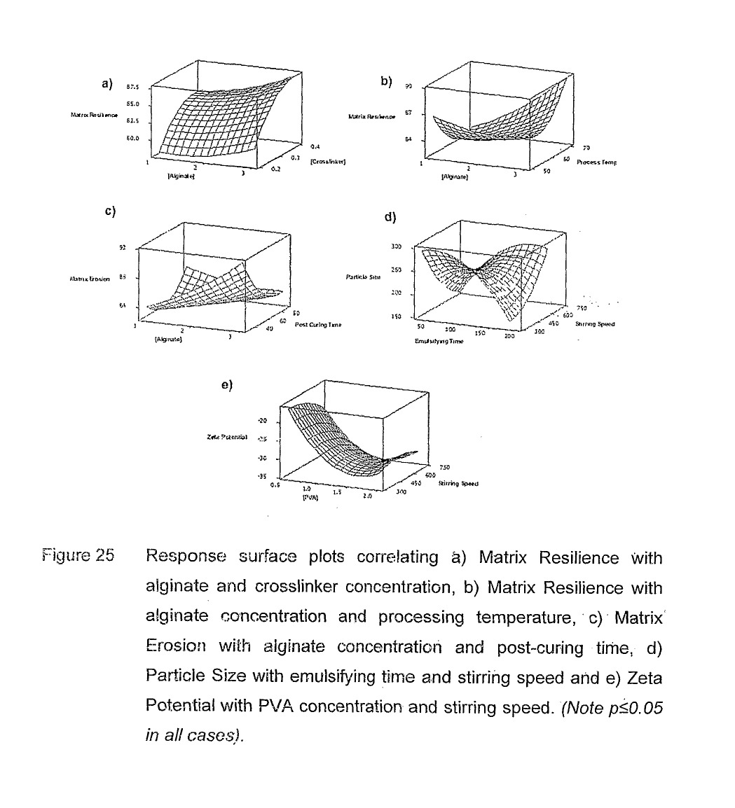

FIG. 25 response surface plots correlating a) Matrix Resilience with alginate and crosslinker concentration, b) Matrix Resilience with alginate concentration and processing temperature, c) Matrix Erosion with alginate concentration and post-curing time, d) Particle Size with emulsifying time and stirring speed and e) Zeta Potential with PVA concentration and stirring speed. (Note p≦0.05 in all cases);

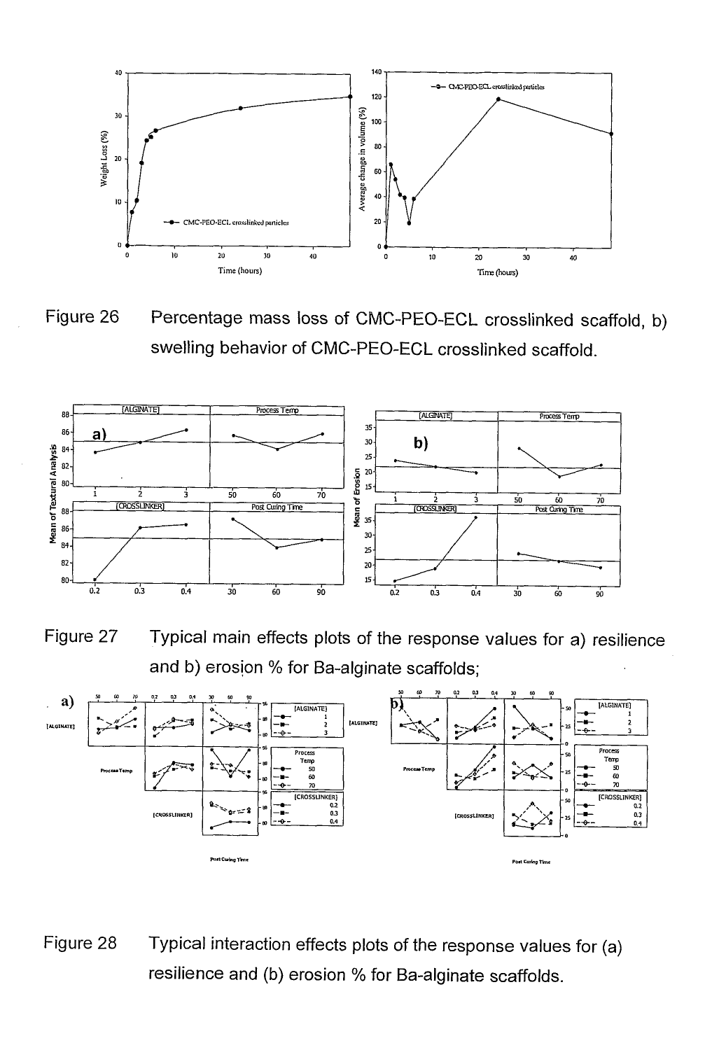

FIG. 26 percentage mass loss of CMC-PEO-ECL crosslinked scaffold, b) swelling behavior of CMC-PEO-ECL crosslinked scaffold;

FIG. 27 typical main effects plots of the response values for a) resilience and b) erosion % for Ba-alginate scaffolds;

FIG. 28 typical interaction effects plots of the response values for (a) resilience and (b) erosion % for Ba-alginate scaffolds;

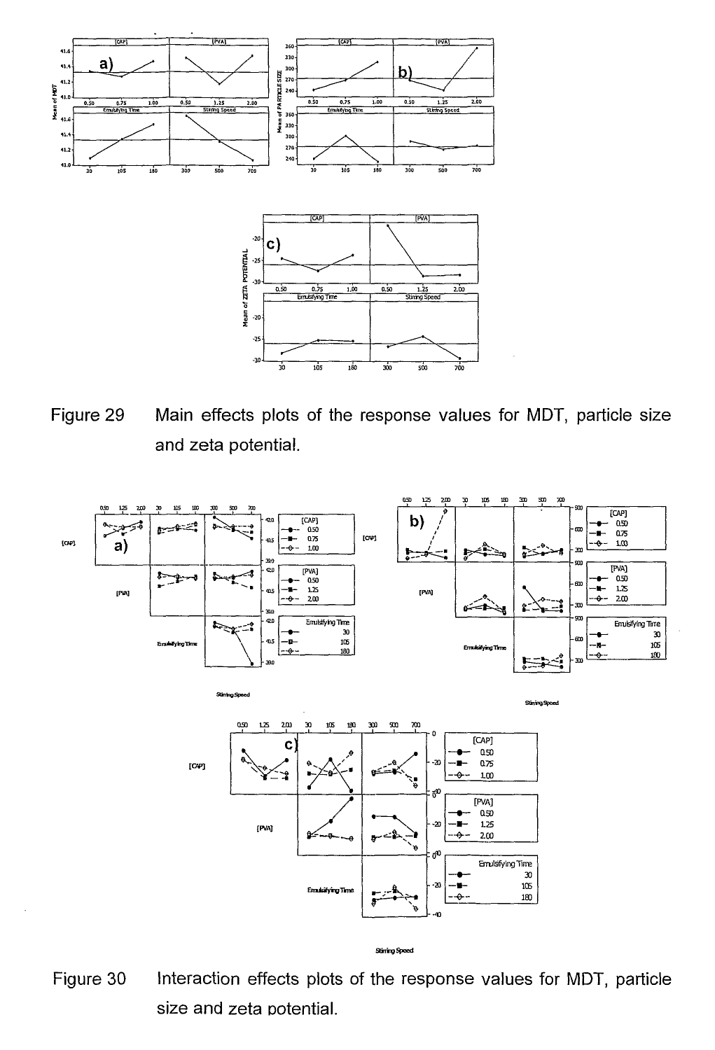

FIG. 29 typical main effects plots of the response values for MDT, particle size and zeta potential;

FIG. 30 interaction effects plots of the response values for MDT, particle size and zeta potential;

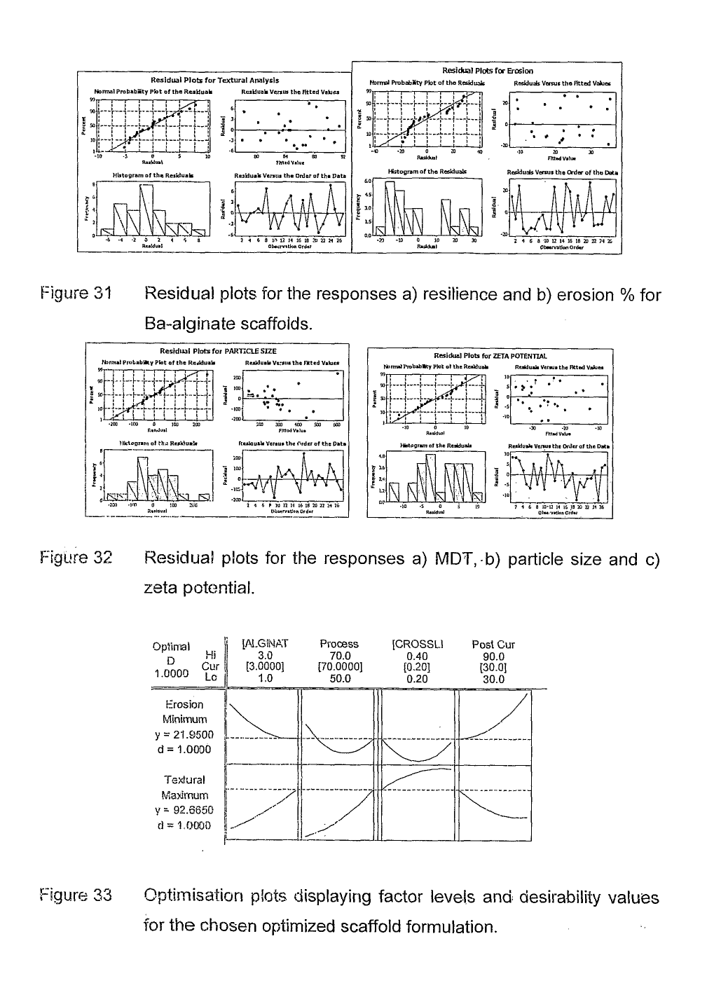

FIG. 31 residual plots for the responses a) resilience and b) erosion % for Ba-alginate scaffolds;

FIG. 32 residual plots for the responses a) MDT, b) particle size and c) zeta potential;

FIG. 33 optimisation plots displaying factor levels and desirability values for the chosen optimized scaffold formulation;

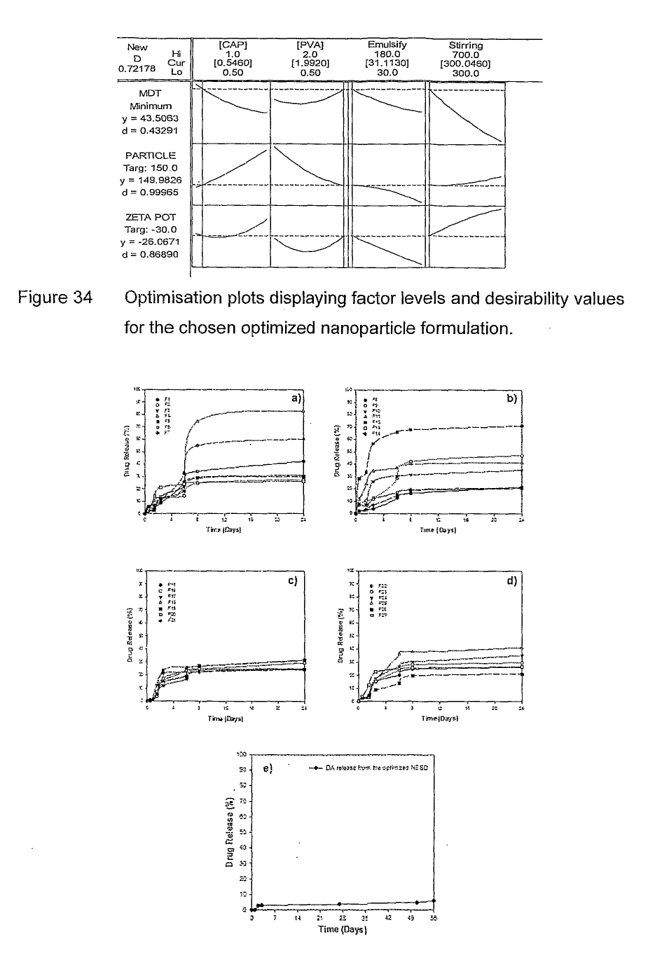

FIG. 34 optimisation plots displaying factor levels and desirability values for the chosen optimized nanoparticle formulation;

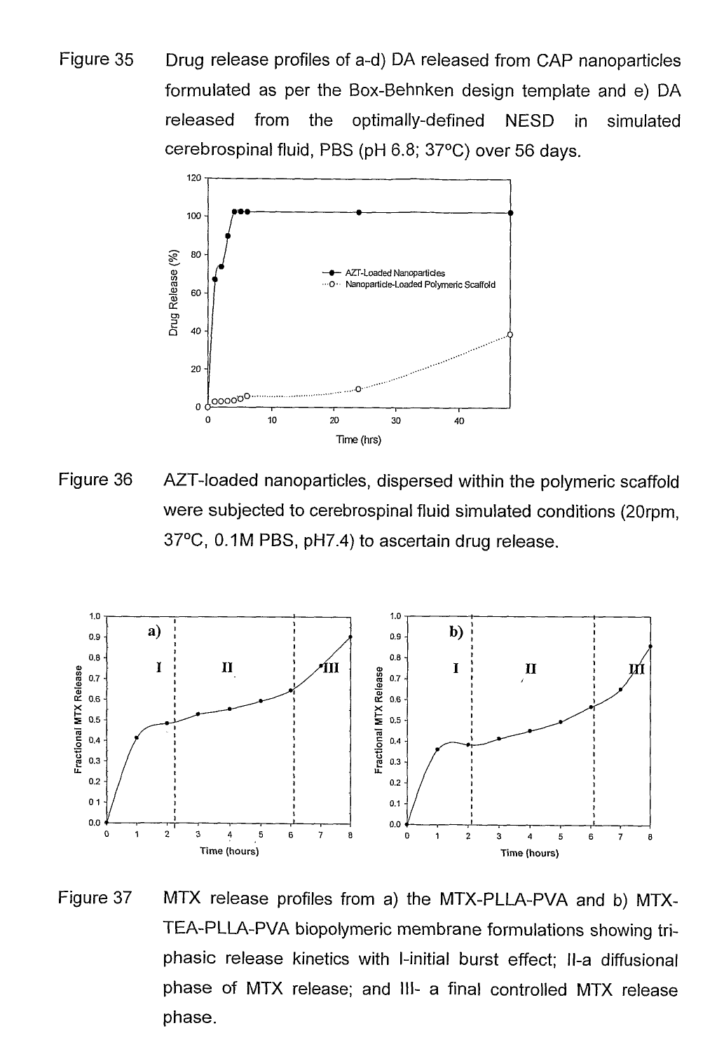

FIG. 35 drug release profiles of a-d) DA released from CAP nanoparticles formulated as per the Box-Behnken design template and e) DA released from the optimally-defined NESD in simulated cerebrospinal fluid, PBS (pH 6.8; 37° C.) over 56 days;

FIG. 36 AZT-loaded nanoparticles, dispersed within the polymeric scaffold were subjected to cerebrospinal fluid simulated conditions (20 rpm, 37° C., 0.1M PBS, pH7.4) to ascertain drug release;

FIG. 37 MTX release profiles from a) the MTX-PLLA-PVA and b) MTX-TEA-PLLA-PVA biopolymeric membrane formulations showing tri-phasic release kinetics with I-initial burst effect; II-a diffusional phase of MTX release; and III-a final controlled MTX release phase;

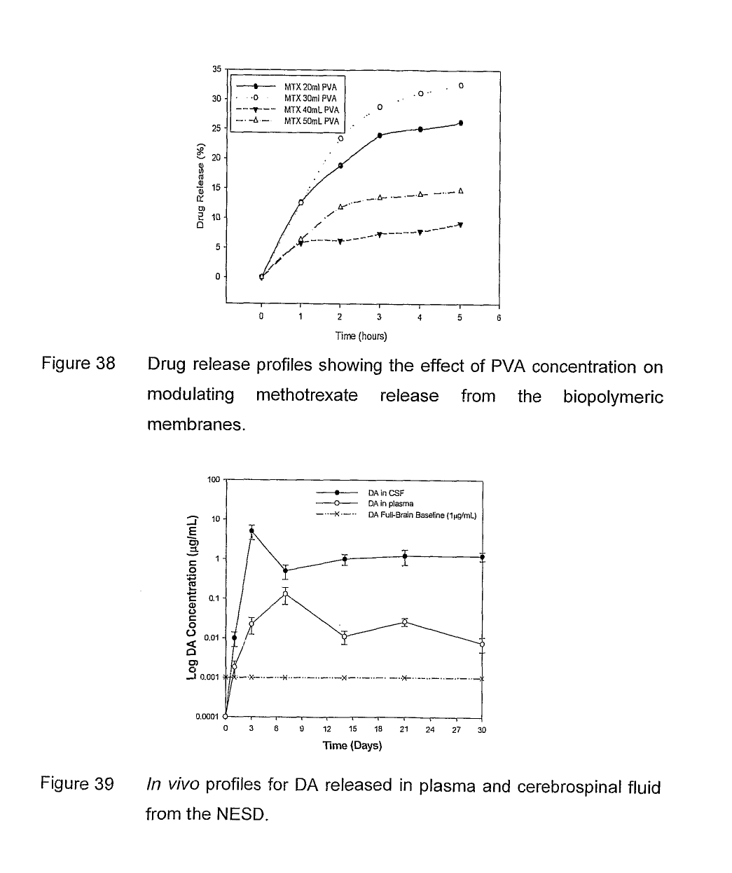

FIG. 38 drug release profiles showing the effect of PVA concentration on modulating methotrexate release from the biopolymeric membranes;

FIG. 39 in vivo profiles for DA released in plasma and cerebrospinal fluid from the NESD;

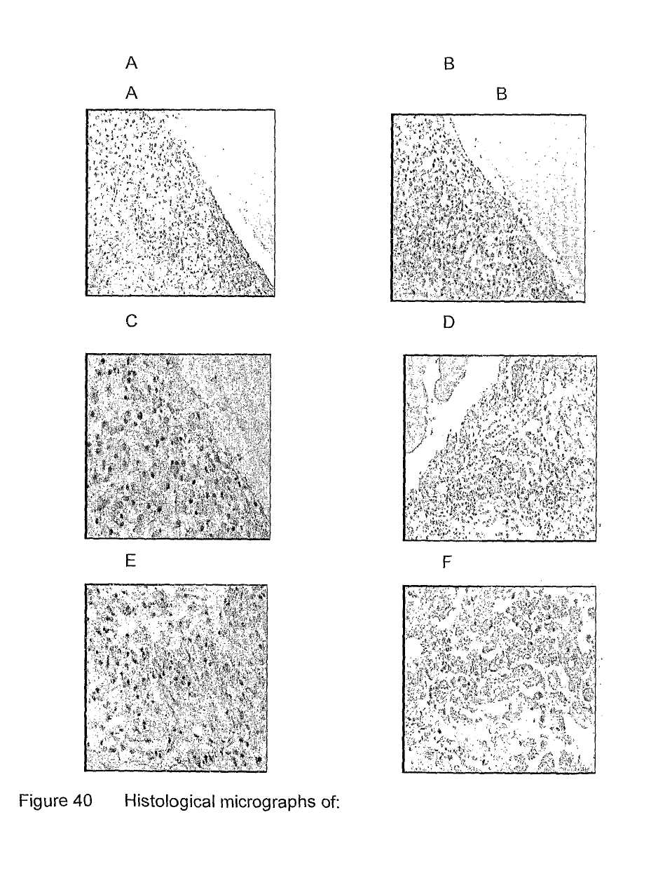

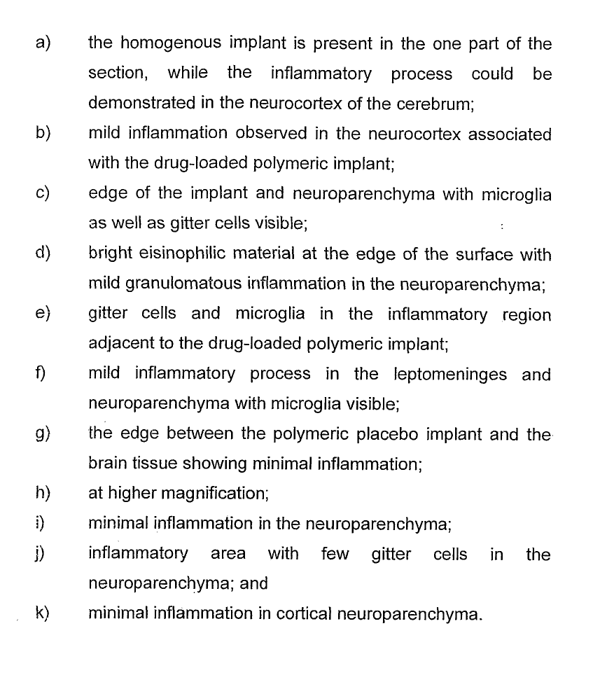

FIG. 40 histological micrographs of:
a) the homogenous implant is present in the one part of the section, while the inflammatory process could be demonstrated in the neurocortex of the cerebrum;
b) mild inflammation observed in the neurocortex associated with the drug-loaded polymeric implant;
c) edge of the implant and neuroparenchyma with microglia as well as gitter cells visible;
d) bright eisinophilic material at the edge of the surface with mild granulomatous inflammation in the neuroparenchyma;
e) gitter cells and microglia in the inflammatory region adjacent to the drug-loaded polymeric implant;
f) mild inflammatory process in the leptomeninges and neuroparenchyma with microglia visible;
g) the edge between the polymeric placebo implant and the brain tissue showing minimal inflammation;
h) at higher magnification;
i) minimal inflammation in the neuroparenchyma;
j) inflammatory area with few gitter cells in the neuroparenchyma; and
k) minimal inflammation in cortical neuroparenchyma.

Each example begins with an exposition on the apparent limitations of previous studies performed in an attempt to address the delivery of a pharmaceutical active compound for site-specific drug delivery and more particularly of polymers and dosage forms according to the invention.

### EXAMPLE 1

### A Biodegradable Cellulose Acetate Phthalate Nano-Enabled Scaffold Device (NESD) for Subarachnoid Implantation for Targeted Dopamine Delivery in Parkinson's Disease.

Drug delivery to the brain remains a highly challenging and essential field of study. Due to the numerous protective barriers surrounding the Central Nervous System (CNS), there is still an urgent need for the effective treatment of patients living with neurodegenerative disorders such as Parkinson's disease (PD) [1]. Parkinson's disease is one of the most common and severely debilitating neurodegenerative diseases [2]. It is characterized by a progressive loss of dopamine neurons in the substantia nigra pars compacta of the brain. This results in the loss of striatal dopaminergic terminals and their ability to store and regulate the release of dopamine. Accordingly, striatal dopamine receptor activation becomes increasingly dependent on the peripheral availability of an exogenously administered dopaminergic agent [3]. As the disease progresses, the patient begins to experience motor abnormalities such as akinesia, resting tremor, and rigidity. The advancement of the disease results in worsening of these symptoms. The Blood-Brain-Barrier (BBB) is a defensive mechanism and therefore the passage of substances into the brain is highly selective. This is a major impediment for drug delivery to the brain as numerous neuroactive drugs are aqueous in nature and therefore unable to penetrate the BBB [4]. Drugs may be delivered systemically as in the case with current drug therapy. However, only a small percentage of drugs reach the brain due to hepatic degradation, and the associated side-effects related to peak-to-trough fluctuation of plasma levels of drug leads to a lack in patient dose-regimen compliance [5]. Currently, levodopa (L-dopa), the levorotatory isomer of dihydroxy-phenylalanine, a metabolic precursor of dopamine is the main therapy used for the treatment of PD. L-dopa is converted into dopamine in the basal ganglia and the current widespread use of L-dopa is to enhance the transport of L-dopa across the BBB. Initial therapy with L-dopa significantly restores the normal functioning of a patient with PD [6]. However a major limitation to the chronic use of L-dopa from conventional oral dosage forms is the resultant ‘end-of-dose wearing-off’ effect where the therapeutic efficacy of each dose of L-dopa resides for shorter periods [7]. Hence, the patient begins to experience motor fluctuations prior to the next dose and therefore the initially prescribed dose is no longer able to effectively manage the symptoms of the disease such as pain, panic attacks, severe depression, confusion and a sense of impending death [8]. Clinicians attempt to overcome this phenomenon by increasing the frequency or quantity of the dose via substituting from immediate release to sustained-release oral formulations to overcome “the wearing off” effect (Sinemet® CR). However, an increased dosing places the patient at a risk of developing side-effects such as dyskinesias [9, 10]. Furthermore, the inclusion of carbidopa with L-dopa tends to exacerbate psychiatric, gastrointestinal and motor side-effects [11, 12]. Polymeric nanotechnology has been investigated for application in targeted cancer therapy [13]. However, there has been minimal progress in the design and institution of nanotechnology for the site-specific treatment of neurodegenerative diseases. Therefore this work explored the design and development of a biodegradable Nano-Enabled Scaffold Device (NESD) to be implanted into the subarachnoid cavity of the brain in order to target the delivery of dopamine for the chronic management of PD. Dopamine was employed as the model drug and thus the peripheral conversion to dopamine that leads to numerous side-effects would be avoided as noted with conventional oral L-dopa delivery systems. The NESD will be able to simplify the treatment of PD, maintain therapeutic levels of dopamine within the brain, reduce the extensive peripheral side-effects experienced by patients and decrease the quantity of dopamine needed as well as the dosing frequency. The inclusion of cellulose acetate phthalate (CAP) nanoparticles into a crosslinked alginate scaffold would facilitate the controlled delivery of dopamine and often higher drug-loading capacities due to the larger surface area to volume ratio as well as facilitating the opening of tight cell-junctions for enhanced BBB penetration [14]. Prototyping technology has created a significant impact in biomedical materials design. Molecular modeling facilitates the design of accurately customized structural models of polymeric devices for various applications [15-20]. This has prompted us to adopt a similar approach to fabricate the NESD with controlled micro-architecture and higher consistency than conventional unsighted techniques. Free-form prototyping technology was used to design the NESD via a three-dimensional (3D) crosslinked alginate scaffold model incorporating CAP nanoparticles. Prototyping provides an alternative that aims to improve the NESD design by employing archetype data manipulation to pre-assemble the complex internal scaffold architectures and nanostructures of the NESD in conjunction with a Box-Behnken statistical design for optimization and an integrated corporeal manufacturing approach that is consistent, reproducible and formulation-specific.

### EXAMPLE 2

### A Biodegradable Polycaprolactone Nano-Enabled Implantable Scaffold (PNIS) for Modulated Site-Specific Drug Release in the Treatment of Aids Dementia Complex

HIV/AIDS is a global concern as the number of people living with the disease is approaching approximately 39.5 million worldwide (UNAIDS/WHO, 2006), with the disease being responsible for 8.7% of deaths in South Africa, as recorded in the last census performed in 2001 (Statistics South Africa). Of the complication associated with HIV/AIDS, AIDS Dementia Complex (ADC) is of particular concern as one third of adults and one half of children living with AIDS are affected by this condition (Bouwer, 1999). ADC is one of the most common and crucial CNS complications of late HIV-1 infection. With little being known of the pathogenesis of the condition, it is a source of severe morbidity, as well as being associated with limited survival (Price, 1998). ADC is responsible for a host of neurological symptoms including memory deterioration; disturbed sleep patterns and loss of fine motor skills (Fernandes et al, 2006). However, cognitive impairment can be reversed by highly active antiretroviral therapy (HAART), or Zidovudine (AZT) monotherapy (Chang et al, 2004). Existing therapies used for the management of ADC are mainly administered via the oral route. However, due to the highly restrictive nature of the Blood Brain Barrier (BBB), bioavailability and therapeutic efficacy of these drugs are poor. Zidovudine (AZT), the current standard for the management of ADC, a nucleoside reverse transcriptase inhibitor (NRTI), has demonstrated the best penetration into the Central Nervous System (CNS), in its class of drugs, being NRTI's. Prior to the introduction of zidovudine (AZT) in 1988, the incidence of ADC in people affected by HIV/AIDS was as high as 53% (1987). However, AZT therapy is hindered by the first pass metabolism, which reduces the bioavailability of this drug. Higher concentrations of this drug are therefore required when used to treat ADC, as high as 1000 mg, as compared to the 600 mg used for HAART therapy, which has been shown to increases the risk of severe aplastic anemia (Aungst, 1999). The poor bioavailability as well as the associated side effects creates the need for localized drug delivery that is capable of bypassing the BBB and systemic circulation, which are responsible for poor bioavailability and many of the side effects experienced with current therapies (Alavijeh et al, 2005). Polymeric nanoparticles used for the controlled delivery of drug were first developed in the 1970's [36]. Drug incorporation into nanosystems is used to achieve site-specific drug delivery, therefore providing better control of drug release, improving improves the efficacy, pharmacokinetics and pharmacodynamics of the drug. Targeted drug delivery improves the therapeutic efficacy of the drug and serves to reduce the quantity of drug administered, thereby minimising side effects experienced due to drug therapy. Drug delivery devices using nanosystems can be manipulated to react in a bioresponsive manner, to provide site-specific drug delivery and to control drug degradation. Nanoparticles are capable of opening tight junctions and are therefore capable of crossing the BBB [32]. Nanoparticles can also be used as carriers for poorly soluble drugs, thereby improving their bioavailability [37, 38, 39]. Polymers with desirable physicochemical and physicomechanical properties can be successfully used to develop nano-enabled implantable devices, which may be used to achieve prolonged release of drug over a desired period of time. Biodegradable polymers such as polycaprolactone (PCL), pectin, and alginate can be used in the design of nano-enabled implantable drug delivery systems, as byproducts of such polymers are biocompatible, nontoxic, and readily excreted from the body [38, 40, 41]. These polymers are non-mutagenic, non-cytogenic and non-teratogenic and are therefore safe for implantation. Such polymers have been employed in simulating a polymer scaffold to deliver drug-loaded polymeric nanoparticles, as these polymers possess desirable mechanical properties and superior drug permeability. The device, comprising of a polymeric scaffold and drug-loaded nanoparticles is intended for intracranial implantation to achieve modulated drug release in a site-specific manner. FIG. 1 illustrates a proposed method of drug delivery into the brain. (38, 40, 41, 42, 43). The development an implantable polymeric, nano-enabled drug delivery device, capable of controlled, site-specific drug delivery will greatly enhance therapy used for the management of ADC [38] (Alavijeh et al, 2005; Tilloy et al, 2006).

### EXAMPLE 3

### A Nano-enabled Biopolymeric Membranous Scaffold (NBMS) for Site-Specific Drug Delivery in the Treatment of Primary Central Nervous System Lymphoma

Advances in biomaterials research has provided solutions for combating numerous challenges posed by various disease conditions [48]. The amalgamation of polymeric science with the pharmaceutical sciences and medicine has led to the development of novel biomaterials for specific applications [49-52]. Despite the progress in the development of such biomaterials a large number of biomaterial-based devices are currently used clinically with unsatisfactory clinical performance [53]. Furthermore, very few synthetic devices are approved by the US Food and Drug Administration (FDA) due to the fact that the time, complexities and attempts to tailor the properties of polymers to complement specific applications are mostly based on trial and error [54]. Therefore there is a need to extend and focus biomaterials research toward economical approaches that may overcome the challenges of designing new biomaterials. Refined approaches such as combinatorial methods, high-throughput experimentation and computational molecular modeling for the development of biomaterials are able to significantly contribute to this area of research by creating opportunities to simulate, investigate, model and predict the structure and properties of newly synthesized biomaterials [55-57]. Computational molecular modeling and structural rationalization techniques are becoming fundamental for the innovative development of biomaterials that were initially unexploited due to the complex nature of biological and pharmacological domains and the expertise and interdisciplinary commitments required to formulate computational models of various phenomena [58-59]. However, the fusion of polymer and pharmaceutical science with computational chemistry has resulted in the incorporation of theoretical chemistry into efficient computer software to gain further insight into the complexity and behaviour of newly synthesized biomaterials in order to justify theoretical concepts when conclusive postulations correlate well with experimental results [60-61]. Computational chemistry employs molecular mechanics and quantum mechanics such as semi-empirical, ab initio and Density Functional Theory (DFT) to predict the molecular structure of biomaterials and compute different molecular descriptors. Computational modeling can be regarded as a third element in the research triad complementing experiment and theory [62, 63]. Ironi and Tentonis [58] employed a computational framework to explore the mucoadhesive potential of sodium carboxymethylcellulose. Polymer-mucin mixtures at varying concentrations underwent standard creep testing and accurate ordinary differential equation models were obtained from the data [58]. Computational modeling functions are best supported by techniques that facilitate the development of predictive models and reveal the molecular structure and underlying physical phenomena governing performance of a biomaterial that would not otherwise be revealed by laboratory experiments [64-66]. Biocompatible and biodegradable polymers in particular have been regarded as suitable materials for developing optimized drug delivery systems with improved therapeutic efficacies, better patient compliance and reduced side-effects [56]. Polymers are a versatile class of materials with well-defined physicochemical and physicomechanical properties [67-70]. Depending on the requirements placed upon a certain material, polymeric drug carriers can be fabricated into various geometries by employing processing methods ranging from implants, stents, grafts, microparticles or nanoparticles or membranes. Combining different polymers is an approach that leads to the formation of a modified polymer provides a broader spectrum for fulfilling the needs drug delivery system. Aliphatic polyesters, such as poly (lactic acid) and their copolymers have been widely used for fabrication of drug delivery devices [7]-73]. In addition, formulations tend to show polyphasic drug release profiles which deviates from the ideal ‘infusion-like’ profile generated by zero-order release formulations [74-76]. Grafting of polyester chains onto hydrophilic backbones would alter the degradation and release properties of the carrier system [77]. Kissel et al [78, 79] successfully formulated a drug delivery system based on a modified polyester fabricated by grafting poly(lactic-co-glycolic acid) onto poly(vinyl alcohol) (PVA-PLGA) or amine modified poly(vinyl alcohol) or sulfobutylated poly(vinyl alcohol) to yield PVA-g-PLGA, DEAPA-PVA-g-PLGA and SB-PVA-g-PLGA respectively. Microparticles prepared from PVA-grafted PLGA also displayed superior encapsulation efficiencies for proteins ranging from 70-90% with yields of approximately 60-85%. Drug release modulation and erosion could be adjusted to meet specific applications when formulated into various drug delivery vehicles such as microparticles, nanoparticles, tablets, implants and membranes with erosion times ranging from hours to weeks [78, 79]. Therefore this study focused on applying computational chemistry as a modeling tool for the rational design of a biopolymeric membrane system for the delivery of methotrexate (MTX). The information obtained from virtual molecular structures and computer models will be used to formulate theoretical postulations on factors such as drug entrapment efficiency and the mechanisms of drug release. MIX was selected as the model drug due to the potential of employing the biopolymeric membrane as an intracranial implant for the treatment of Primary Central Nervous System Lymphoma [80]. Intra-tumoral and site-specific drug delivery strategies have gained momentum recently as a promising modality in cancer therapy. The reason is that most chemotherapeutic agents used for the treatment of brain tumors cannot cross the blood-brain barrier when given intravenously; hence this necessitates frequent and higher dosing of cancer drugs to achieve optimum therapeutically active concentrations at the tumor site. However, albeit these higher drug concentrations, local tumor recurrences are common and detrimental side-effects make cancer treatment unbearable to most patients. Primary Central Nervous System Lymphoma (PCNSL), once a rare type of a brain tumor and a subject of individual case reports now afflicts many people each year. The tumor resides behind the intact blood-brain barrier and can completely regress with either corticosteroid or cranial irradiation only to recur. Unlike malignant gliomas appropriate treatment may result in prolonged survival and or even cure. High dose of methotrexate (MTX) (8 g/m²) as part of the initial therapeutic regimen has been shown to provide dramatic benefits compared with radiotherapy alone. However these benefits are associated with chemotherapy-related toxicity. Therefore site-specific delivery of MTX may be beneficial in achieving a more effective therapeutic outcome and improving patient compliance.

### 2. Materials and Methods

### 2.1. Materials

The following materials were used for the NESD development: Alginate (Protanal® LF10/60; 30% mannuronic acid, 70% guluronic acid residues) was purchased from FMC Biopolymer (Drammen, Norway). Calcium gluconate [(HOCH₂ (CHOH)₄COO)₂Ca], barium chloride (BaCl₂), cellulose acetate phthalate (CAP) (M_{w}=49,000 g/mol.), poly(vinyl alcohol) (PVA), acetone, methanol and dopamine hydrochloride (DA) (M_{w}=189.64 g/mol.) were purchased from Sigma Aldrich (St. Louise, Mo., USA). Double deionized water was obtained from a Milli-Q water purification system (Milli-Q, Millipore, Billerica, Mass., USA). Solid phase extraction procedures were performed with Oasis® HLB cartridges purchased from Waters® (Milford, Mass., USA). Healthy adult Sprague Dawley rats were used for the in vivo release study weighing 400-500 g and housed in groups of three per cage under controlled environment (20±2° C.; 65±15° C. % relative humidity) and maintained under 12:12 h light: dark cycle. Theophylline was used as an internal standard during UPLC analysis. All solvents used for UPLC analysis were of analytical grade.

The following materials were used for the PNIS development: Biodegradable, biocompatible polymers, alginate, pectin, polycaprolactones and sodium carboxymethylcellulose (NaCMC), were purchased from Sigma, (Johannesburg, South Africa), and utilized to synthesize nanoparticles and the polymer scaffold. Calcium chloride (CaCl₂), barium chloride (BaCl₂) and sodium thiosulphate salts were used as crosslinking agents in the synthesis of nanoparticles and the polymer scaffold. Polyvinyl alcohol was required in the synthesis of the nanoparticles, serving as a surfactant. Solvents used during the study include dimethyl sulfoxide (DMSO), (Sigma, South Africa) and distilled water. Alginate sodium (Protanal® LF) was purchased from FMC Biopolymer (Drammen, Norway). Calcium gluconate [(HOCH₂(CHOH)₄COO)₂Ca] cellulose acetate phthalate (CAP), acetone, poly(vinyl alcohol) (PVA), methanol and dopamine hydrochloride (DA) were all purchased from Sigma (Johannesburg, South Africa).

The following materials were used for the NBMS development: Methotrexate (MTX) (model drug) and stannous octoate (catalyst) (Tin (II) 2-ethylhexanoate) were purchased from Sigma Aldrich (St Louis, Mo., USA). Poly(vinyl alcohol) (PVA; M_{w}=49,000 g/mol) and triethanolamine (TEA) (plasticizer) was purchased from Saarchem (Krugersdorp, South Africa). Poly (L-lactic acid) (PLLA; Resomer® grade R203H) was purchased from Boehringer Ingelheim (Ingelheim, Germany) and dimethyl-sulphoxide (DMSO) (solvent), reagent grade acetone and methanol (non-solvent blend) were purchased from Rochelle Chemicals (Johannesburg, South Africa). The rationale for using folic acid (FA) as a model drug is as follows, FA serves as a metabolite in biochemical pathways. It undergoes reduction catalysed by an enzyme dihydrofolate reductase (DHFR) to give dihydrofolic acid which is subsequently transformed to folate co-factors. The folate co-factors serve the important biochemical function of donating one-carbon unit at various levels of oxidation which leads to the synthesis of amino acids, purines, and DNA. MTX is a′ FA antagonist that binds to the active catalytic site of DHFR, interfering with the synthesis of the reduced form that accepts one-carbon unit. Lack of this cofactor interrupts the synthesis of thymiylate, purine, nucleotides, and the amino acids serine and methionine, thereby interfering with the formation of DNA and RNA and proteins. The enzyme binds MTX with high affinity and virtually no dissociation of the enzyme-inhibitor complex occurs at pH 6.0 (inhibition constant=μmol/L) [48]. MTX inhibits FA from binding to DHFR and blocks the intermediary metabolic step of proliferating cancerous cells [1]. MTX, N-[4-{[2,4-d]amino-6-pteridinyl)-methyl]methyl amine}benzoyl]glutamic acid is a structural analogue of FA N-(p-{2-amino-4-hydroxypyramido [4,4-b]pyrazi-6-yl) methylamino]benzyol}glutamic acid (FIG. 2).

### 2.2. Computer-Aided Prototyping of the Devices

### 2.2.1. The NESD Device

The implicit design of the nano-enabled scaffold device (NESD) required customization of the crosslinked alginate scaffold for embedding the DA-loaded CAP nanoparticles with the ability to support bioadhesion and the physicomechanical stability for intracranial implantation of the device. CAP and [(HOCH₂(CHOH)₄COO)₂Ca]-crosslinked alginate were selected for producing the nanoparticles and scaffold components of the NESD respectively. The crosslinked scaffold was subsequently cured in a BaCl₂ solution as a secondary crosslinking step. The componential NESD properties were modulated through computational prototyping to produce a viable scaffold embedded with stable CAP nanoparticles. The fundamental design parameters were pivoted on the polymer assemblage, curing methods, surface properties, macrostructure, physicomechanical properties, nanoparticle fixation and biodegradation of the NESD. In order to incorporate fine control within the complexities of three-dimensional (3D) design, the physical properties of the crosslinked alginate scaffold such as the pore size, shape, wall thickness, interconnectivity and networks for nanoparticle diffusion was regulated to produce a 3D prototype NESD model. The NESD topography was predicted for intracranial implantation with pre-defined micro-architecture and physicomechanical properties equilibrating frontal lobe brain tissue as the site of implantation to provide mechanical support during sterilizability prior to function. A suppositional 3D graphical model with potential inter-polymeric interactions during formation was generated on ACD/I-Lab, V5.11 Structure Elucidator Application (Add-on) biometric software (Advanced Chemistry Development Inc., Toronto, Canada, 2000) based on the step-wise molecular mechanisms of scaffold and nanoparticle formation, polymer interconversion and DA-loaded nanoparticle fixation as envisioned by the chemical behaviour and physical stability. A combination of a computationally rapid Neural Network (NN) and a modified Hierarchal Organization of Spherical Environments (HOSE) code approach were employed as the fundamental algorithms in designing the prototype NESD. The associated energy expressions were chemometrically designed based on the assumption of the scaffold behaving initially as a gel-like structure with higher states of combinatory energy for the complete NESD.

### 2.2.2. The NBMS Device

Computational and molecular structural modeling was performed to deduce a hypothesized chemical structure and potential inter-polymeric interaction during membrane balance and layering. Semi-empirical, ab initio and Density Functional Theories (DFT) of molecular and quantum mechanics was used to generate predictions of the molecular structure of the materials and compute various molecular attributes based on the inherent interfacial phenomena underlying the formation of the biopolymeric membranes prepared by the immersion precipitation technique. Models and graphics based on the step-wise molecular mechanism of membrane formation, polymer interconversion and grafting and drug chelation as envisioned by the chemical behaviour and stability were generated on ACD/1-Lab, V5.11 (Add-on) software (Advanced Chemistry Development Inc., Toronto, Canada, 2000).

### 2.3. A Box-Behnken Design Strategy for Device Preparation and Optimization

### 2.3.1. The NESD Device

Two separate quadratic 4-factor Box-Behnken statistical experimental designs were constructed in order to produce concise experimental batches of the crosslinked alginate scaffold and DA-loaded CAP nanoparticles as the solitary components of the NESD. The scaffold and nanoparticles were optimized within each design matrix in constraints of maximizing the scaffold Matrix Resilience in the hydrated state, minimizing the scaffold Matrix Erosion, maximizing the Mean Dissolution Time (MDT) of DA from the CAP nanoparticles and minimizing the Zeta Potential and Particle Size of the CAP nanoparticles. The upper and lower limits of the independent formulation factors, the responses selected and the optimization constraints for the crosslinked alginate scaffold and CAP nanoparticles are listed in Table 1. Quadratic relationships linking the independent formulation factors and responses were generated, and the constituents of the NESD were optimized under pre-determined constraints intimated by the initial prototyping technology employed. The study design was generated and analyzed using Minitab® V15 software (Minitab® Inc, Pa., USA) with two separate formulation design templates for the crosslinked alginate scaffold and CAP nanoparticles with a total of 27 experimental runs for each blueprint.

TABLE 1
Independent formulation factors and responses selected for NESD
preparation and optimization

| | | | |
|---|---|---|---|
| Independent Factors | Lower | Levels | Upper |

Crosslinked alginate scaffold

| | | | |
|---|---|---|---|
| Alginate (% ^{W}/_{V}) | 1 | | 3 |
| Calcium gluconate (% ^{W}/_{V}) | 0.2 | | 0.6 |
| Temperature (° C.) | 50 | | 70 |
| Post-curing time (min) | 30 | | 90 |
| Responses | Lower | Upper | Objective |
| Matrix Resilience (%) | 86 | 94 | Maximize |
| Matrix Erosion (%) | 3 | 59 | Minimize |

DA-loaded CAP nanoparticles

| | | |
|---|---|---|
| CAP (g) | 0.5 | 1 |
| PVA (% ^{W}/_{V}) | 0.5 | 2 |
| Stirring speed (rpm) | 300 | 700 |
| Emulsifying time (min) | 30 | 180 |
| Responses | Lower | Objective |
| Mean Dissolution Time (MDT) | 38 | Maximize |
| Zeta Potential (mV) | −20 | Maximize |
| Particle Size (nm) | 150 | Minimize |

### 23.1.1. Corporeal Assembly of the NESD

Production of the NESD required the initial componential preparation and optimization of the crosslinked alginate scaffold and the DA-loaded CAP nanoparticles. Once the two components were optimized the DA-loaded CAP nanoparticles were incorporated via intermittent blending and lyo-fusion (spontaneous freezing followed by lyophilization) into the [(HOCH₂(CHOH)₄COO)₂Ca]-crosslinked and BaCl₂-cured alginate scaffold.

### 2.3.1.2. Preparation of the Crosslinked Alginate Scaffold

A 2% w/v, alginate solution in deionized water (Milli-DI® Systems, Bedford, Mass., USA) was prepared at 50° C. and a primary 0.4% w/v [HOCH₂(CHOH)₄COO]₂Ca-crosslinking solution was added and agitated until a homogenous mixture was obtained. The resulting ‘gel-like’ solution was then placed in Teflon moulds and lyophilized for 24 hours at 25 mtorr [21]. Thereafter the lyophilized structures were immersed in a secondary 2% w/v BaCl₂ crosslinking solution for 3 hours as a curing step followed by a further lyophilization phase of 24 hours at 25 mtorr (Virtis, Gardiner, N.Y., USA). The resultant cured scaffolds were removed from the moulds, washed with 3×100 mL deionized water to leach out unincorporated salts and air-dried under an extractor until a constant mass was achieved. All formulations were prepared in accordance with a Box-Behnken experimental design template.

### 2.3.1.3. Preparation of the DA-Loaded Cap Nanoparticles

Nanoparticles were prepared using an adapted emulsification-diffusion technique [22], in accordance with a Box-Behnken experimental design template generated. Briefly, 500 mg of CAP and 50 mg of DA were dissolved in a binary solvent system of acetone and methanol in a 3:7 ratio (100 mL). A 1% w/v PVA solution was then added as a surfactant. The solution was agitated for 30 minutes using a magnetic stirrer set at 700 rpm. A sub-micronized o/w emulsion was spontaneously formed due to immediate reduction of the interfacial tension with rapid diffusion of the binary organic solvent system into the aqueous phase known as the Marangoni Effect [23]. Excess solvent was evaporated using a Rotavap (Rotavapor® RII, Switzerland) maintained at 60° C. for 1 hour and the resulting concentrate was centrifuged (Optima® LE-80K, Beckman, USA) at 20,000 rpm for 20 minutes. The sedimentary layer containing CAP nanoparticles was then removed and lyophilized for 24 hours at 25 mtorr to obtain a free-flowing powder for incorporation into the crosslinked alginate scaffold via lyo-fusion.

2.3.1.4. Assimilation of the Crosslinked Scaffold and Cap Nanoparticles into the NESD

The NESD was assembled by a lyo-fusion process. Briefly, the optimally defined DA-loaded CAP nanoparticles (200 mg) were placed into moulds containing a [HOCH₂(CHOH)₄COO]₂Ca-alginate solution (2 mL) obtained in accordance with set optimization constraints. The mixture was agitated and spontaneously frozen at −70° C. for 24 hours. The frozen structures were lyophilized for 48 hours at 25 mtorr and thereafter immersed in a 2% w/v BaCl₂ crosslinking solution for 3 hours as a curing step followed by a further lyophilization phase of 24 hours at 25 mtorr to induce fusion of the DA-loaded CAP nanoparticles and the crosslinked and cured alginate scaffold.

### 2.3.2. The PNIS Device

### 2.3.2.1. Preparation of AZT-Loaded Polymeric Nanoparticles

Nanoparticles were prepared using a controlled gelification of alginate approach, whereby sodium alginate and AZT were dissolved in distilled water and stirred at maximum speed. A 90% w/v CaCl₂ solution was then added to the alginate-AZT solution in a drop-wise manner over 30 min to facilitate crosslinking. A 0.05% w/v pectin solution and a 1% w/v PVA solution were then added to the crosslinked suspension to stabilize the nanoparticle suspension. Nanoparticles were then centrifuged to further precipitate nanoparticles, dried at ambient temperatures and lyophilized (Vits, Gardiner, N.Y., USA) for 24 hours to obtain a free-flowing powder.

### 2.3.2.2. Preparation of a Cellulose/Caprolactone Polymeric Scaffold

Sodium carboxymethylcellulose (NaCMC), epsilon-caprolactone (ECL) and polycaprolactone (PCL) were dissolved in deionized water. AZT-loaded nanoparticles were evenly dispersed within the polymer solution, which was then crosslinked with a 10% w/v CaCl₂ and BaCl₂ solution to prepare the polymeric scaffold. Crosslinked scaffolds were dried at ambient temperature and lyophilised to remove residual water. The scaffolds were then exposed to gamma radiation to further facilitate crosslinking. Another batch of scaffolds were produced using a combination of PCL and ECL in varying concentrations, which were dissolved in acetone, and allowed to evaporate at room temperature.

2.3.2.3. Preparation of a Ba-Alginate Scaffold Alginate (2 g) was dissolved in 100 mL deionized water (Milli-DI® Systems, Bedford, Mass., USA) at 50° C. A 100 mL of Ca-gluconate solution (0.4% w/v) was added to the polymeric solution and agitated until a homogenous mixture was obtained. The resultant mixture was then placed in teflon moulds and lyophilized for 24 hours (Virtis, Gardiner, N.Y., USA) (Zmora et al., 2002). Thereafter the lyophilized scaffolds were placed in BaCl₂ (2% w/v) solutions for 3 hours as a post-curing step followed by lyophilization for a further 24 hours. The resulting scaffolds were removed from the moulds, washed in deionized water to leach out any remaining salts and air-dried under an extractor to constant mass.

### 2.3.3. The NBMS Device

MTX-loaded biopolymeric membranes were fabricated by layered hydrophile-lipophile conjugation and graft co-polymerization of PLLA and PVA with and without the addition of the amphiphile TEA (PLLA-PVA and TEA-PLLA-PVA) employing stannous octoate as a catalyst at a reaction temperature of 150° C. TEA was added due to it's relatively balance interphase absorption and was reacted with the modified co-polymer to induce backbone activation for the addition of model drug methotrexate (MTX). Phase separation was achieved by an immersion precipitation technique. Briefly, homogenous solutions of PLLA and PVA (10% w/v) were blended after solubilization in DMSO. The polymers were reacted in a ratio of 1:1.75 (15:20 mL) PLLA/PVA in the presence of stannous octoate at 150° C. for 1 hour. Thereafter, 2.5 mL TEA was added to the polymeric solution and the reaction was allowed to proceed for a further 1 hour. MTX (15 mg) dissolved in 0.5 mL DMSO was added to 2.5 mL of the composite polymeric solutions and casted on a glass petri dish (15 mm in diameter) and then immersed in a mixed non-solvent system comprising acetone:methanol in a ratio of 1:1. The resultant biopolymeric membranes were recovered after 24 hours from the coagulation bath and allowed to dry at room temperature (21±0.5° C.) prior to further characterization. All reactions were performed with purified core molecules and monomers. Phase separation and subsequent membrane formation was highly dependent on the concentration of PVA and the volume ratio of PLA/PVA (Table 2). Phase separation did not occur when the polymer volume ratio was less than 1:1.3 and greater than 1:3.3 PLA/PVA. Similarly, PVA concentrations less than 10% w/v and greater than 20% w/v did not favour phase separation. Biopolymeric membranes formed outside the limits degraded rapidly and released the entire drug within 24 hours (Table 3).

TABLE 2
Upper and lower formulation variables

| | PVA concen- | PVA volume | Triethanol- | Drug |
|---|---|---|---|---|
| Limits | tration (% ^{W}/_{V}) | ratio (mL) | amine (mL) | loading |
| Lower limit | 8 | 20 | 0.5 | 10 mg |
| Upper limit | 10 | 50 | 4.5 | 30 mg |

TABLE 3
Experimental design template for the various
statistically generated formulations

| Formulation | | VOLUME RATIO | |
|---|---|---|---|
| [TEA] | [PVA] | (PVA) | |
| 1 | 10 | 50 | 2.5 |
| 2 | 20 | 35 | 4.5 |
| 3 | 10 | 35 | 4.5 |
| 4 | 15 | 35 | 2.5 |
| 5 | 15 | 50 | 0.5 |
| 6 | 20 | 50 | 2.5 |
| 7 | 10 | 35 | 0.5 |
| 8 | 15 | 35 | 2.5 |
| 9 | 15 | 20 | 0.5 |
| 10 | 20 | 20 | 2.5 |
| 11 | 15 | 20 | 4.5 |
| 12 | 15 | 35 | 2.5 |
| 13 | 15 | 50 | 4.5 |
| 14 | 10 | 20 | 2.5 |
| 15 | 20 | 35 | 0.5 |

2.4. Determination of the Particle Size and Zeta Potential of the Various Nanoparticle Formulations from the NESD, PNIS and NBMS Devices

In order to assess the physical stability of the drug-loaded nanoparticles produced, the zeta potential value was analyzed using a Zetasizer NanoZS instrument (Malvern Instruments Ltd, Malvern, Worcestershire, UK) to measure the particle surface charge. DA-loaded CAP nanoparticle samples (1% w/v) produced in accordance with the Box-Behnken formulation design template was appropriately suspended in deionized water as the dispersant, passed through a membrane filter (0.22 μm, Millipore Corp., Bedford, Mass., USA) to maintain the number of counts per second in the region of 600, and placed into folded capillary cells. The viscosity and refractive index of the continuous phase were set to those specific to deionized water. Particle size measurements were performed in the same manner using quartz cuvettes. Measurements were taken in triplicate with multiple iterations for each run in order to elute size intensity and zeta potential distribution profiles. Analysis of particle size and zeta potential of the PNIS and NBMS devices were also undertaken with a ZetaSizer NanoZS to determine the average sizes and size distribution of the nanoparticles produced, employing dynamic light scattering. Zeta potential was employed to determine overall surface charge distribution and stability of the nanoparticles. Nanoparticles were dispersed in phosphate buffered saline (PBS) at pH 7.4. The dispersion was then analysed over a designated time period to observe degradation and solubilization behaviour of the nanoparticles.

2.5. Assessment of Drug Entrapment Efficiency within the Nanoparticles

### 2.5.1. The NESD and PIM Devices

In order to assess the entrapment efficiency of drug within the nanoparticles, post-lyophilized powdered samples were accurately weighed and completely dissolved in phosphate-buffered saline (PBS) (pH 6.8; 37° C.). The drug content was analyzed by UV spectrophotometry (Hewlett Packard 8453 Spectrophotometer, Germany) and computed from a standard linear curve of drug in PBS (pH 6.8; 37° C.) (R²=0.99). Equation 1 was utilized to compute the Drug Entrapment Efficiency (DEE).

$$DEE \% = \frac{{D}_{a}}{{D}_{t}} \times 100 \qquad Equation 1$$

Where DEE % is the drug entrapment efficiency, Dₐ is the actual quantity of drug (mg) measured by UV spectroscopy and Dᵣ is the theoretical quantity of drug (mg) added in the formulation.

### 2.5.2. The NBMS Device

DEE analysis of the biopolymeric membrane was performed by re-dissolving membrane samples in 100 mL PBS (pH 7.4; 37° C.) and subsequently determining the quantity of MTX entrapped using a previously constructed standard linear curve generated at the maximum UV wavelength of λ₃₀₃ₙₘ, for MTX (CECIL 3021 Spectrophotometer, Cecil Instruments, Cambridge, England). The DEE value was calculated employing Equation 2.

$$DEE = \frac{( {M}_{i} - {M}_{d} )}{{M}_{i}} \times 100 \qquad Equation 2$$

Where, Mᵢ is the initial mass of MTX dissolved in the casting polymer solution and M_{d} is the mass of MTX quantified in the media after membrane samples were completely dissolved.

The highest drug loading was achieved when 30 mg of FA was incorporated into the system. Incorporation of drug amounts <30 mg also resulted in membranes with acceptable physical and chemical properties. Increasing drug concentrations >30 mg compromised the physicochemical properties of the formulation resulting in formulations with rapid dissolution/degradation kinetics (2 days) in phosphate buffered saline (PBS, pH 7.4) at 37° C. FIGS. 3 and 4 illustrate DEE of various FA-loaded NBMS devices. The first two digits after PTEA designate the volume of PVA and the last two digits designate amount of triethanolamine (TEA). TEA played a role as a drug binding motif with dendrimeric qualities capable of binding multiple drug molecules. Its inclusion in the formulations improved DEE. The minimum amount, of TEA that showed significant improvement in DEE was 0.5 mL and the highest amount that could be included in the formulation was 4.5 mL. DEE increased with increasing amount of TEA. Amounts of TEA outside the specified limits did not show any significant effects on the DEE of the formulations. Furthermore, the presence of TEA in the formulation contributed to drug modulating effects. Quantities of TEA outside the specified limits appeared to not have any significant effect on the formulation. Highest drug-loading was achieved with 30 mg of FA or MTX. Higher drug concentrations resulted in rapid polymeric membrane degradation (2 days) in phosphate buffered saline (PBS) (pH 7.4; 37° C.).

### 2.6. Morphological Characterization of the Devices

Morphological characterization of the crosslinked alginate scaffold and DA-loaded CAP nanoparticles was instituted. The shape, size homogeneity and possible degree of aggregation were identified for the DA-loaded and native CAP nanoparticles. In addition the scaffold parameters such as the micro-structure, pore length, pore distribution and inter-pore wall thickness was also examined. The surface morphology of the cured and un-cured crosslinked alginate scaffolds were also characterized to assess the influence of crosslinking and subsequent curing on potential surface morphological transitions (N=10). SEM (JEOL, SEM 840, Tokyo Japan) was employed and photomicrographs were captured at various magnifications for analyzing the scaffold and nanoparticle samples that were prepared after sputter-coating with carbon or gold. The nanoparticle size and shape was also explored using Transmission Electron Microscopy (TEM) (JEOL 1200 EX, 120 keV) for higher definition and resolution. Samples were prepared by placing a dispersion of nanoparticles in ethanol on a copper grid with a perforated carbon film followed by evaporation and viewing at room temperature (N=10). SEM was also employed on samples of the PNIS and NBMS devices that were coated with carbon and gold-palladium, after which they were visualized under different magnifications. Various photomicrographs were attained under an electrical potential of 15 kV by scanning fields selected at different magnifications. Photomicrographs were obtained and analyzed to study surface morphology. The degree of entanglement, network density and porosity of the polymeric scaffolds was determined using the photomicrographs obtained. Nanoparticles were also analyzed using cryo-TEM to assess the size and morphology of individual particles produced.

### 2.7. Physicomechanical Characterization of the Devices

### 2.7.1. The NESD Device

One of the key approaches to intricate crosslinked polymeric scaffold engineering is the assessment of the physicomechanical properties of the scaffold matrix following 3D prototyping and prior to sterilization and intracranial implantation. The micro-mechanical properties of the crosslinked alginate scaffold may directly influence the ability of the CAP nanoparticles to fuse and migrate during preparation, sterilization and function. Textural profile analysis was therefore conducted to characterize the 3D salient core regions of the crosslinked alginate scaffold using a Texture Analyzer (TA.XTplus Stable Microsystems, Surrey, UK) in terms of the scaffold Matrix Resilience. Hydrated samples of the crosslinked alginate scaffold were analyzed. Serial Force-Time profiles were sufficient to perform the necessary computations of Matrix Resilience (N=5). The parameter setting employed comprised a Pre-Test Speed=1.0 mm/sec, a Test Speed and Post-Test Speed=1.5 mm/sec, 50% Strain under a Compressive Test Mode with a Trigger Force of 0.05N.

### 2.7.2. The PNIS Device

A Texture Analyzer was also used to establish various stress-strain parameters of the polymeric scaffold. Samples in both the hydrated and unhydrated states were assessed. Force-Distance and Force-Time profiles were obtained and matrix resilience and hardness were calculated.

### 2.7.3. The NBMS Device

Textural profile analysis was employed for all physicomechanical investigations. The bi-axial extensibility was determined from Force-Distance profiles generated on a Texture Analyzer equipped with a 2 mm flat cylindrical probe, a 5 kg Ioadcell and Texture Exponent V3.2 software for data processing. The method involved securing the biopolymeric membranes on a ring assembly with a 5 mm diameter central hole using a secure raised platform (FIG. 5). The centralized test probe was then lowered and embedded onto the membrane surface according to the relevant test parameter settings for determining the biopolymeric membrane toughness and bi-axial extensibility as specified in Table 4.

TABLE 4
Test parameters employed bi-axial extensibility
testing of the biopolymeric membrane

| Parameter | Setting | |
|---|---|---|
| Test mode | Compression | |
| Pre-test speed | 1.00 | mm/sec |
| Test speed | 1.00 | mm/sec |
| Post test speed | 1.00 | mm/sec |
| Trigger mode | Distance | |
| Distance | 10 | mm |

| | |
|---|---|
| Trigger force | 0.5000N |

Biopolymeric membranes with desirable physicochemical and physicomechanical properties were formed by ensuring that the ratio of PVA:SnOct was maintained at 1:10. Stannous octoate was used as a catalyst. (esterification reagent) to facilitate the reaction between PVA and PLA. Keeping the catalyst at constant volume resulted in the formation of biopolymeric membranes with rapid degradation and drug release kinetics.

### 2.8. Determination of Polymeric Structural Variations Due to Device Formation

2.8.1. The NESD Device The molecular structure of native CAP, DA and the CAP nanoparticles produced were analyzed using Fourier Transmission Infrared (FTIR) spectroscopy to elucidate any variations in vibrational frequencies and subsequent polymeric structure as a result of DA-CAP interaction during nanoparticle formation. Molecular structural changes in the CAP backbone may alter the inherent chain stability and therefore affect the physicochemical and physicomechanical properties of the selected polymer type for the intended purpose. Samples of DA-free and DA-loaded CAP nanoparticles were blended with potassium bromide (KBr) in a 1% w/w ratio and compressed into 1×13 mm disks using a Beckmann Hydraulic Press (Beckman Instruments, Inc., Fullerton; USA) set at 8 tons. The sample disks were analyzed in triplicate at high resolution with wavenumbers ranging from 4000-400 cm⁻¹ on a Nicolet Impact 400D FTIR Spectrophotometer coupled with Omnic FTIR research grade software (Nicolet Instrument Corp, Madison, Wis., USA). FTIR was also utilized for the PNIS and NBMS devices to establish whether a new compound had been produced. This was established by comparing the chemical structure of the parent compounds with that of the compounds produced to determine whether structural transitions had occurred during the preparation process.

### 2.9. Componential Thermal Characterization of the Devices

### 2.9.1. The NESD Device

The inherent and sequential transient thermal behaviour of polymers may influence the physicochemical and physicomechanical properties as well as the final performance of the system [24]. Temperature Modulated Differential Scanning calorimetry (TMDSC) was therefore performed to provide a distinct interpretation of the polymeric thermal transitions with improved sensitivity and the ability to separate reversible glass transition temperatures (T_{g}) that have minimal changes in heat capacity (ΔH) from overlapping non-reversible relaxation endotherms [25-27]. Thermal analysis was therefore undertaken on the DA-loaded CAP nanoparticles, the crosslinked alginate scaffold and the assimilated NESD in order to assess thermal behavior using TMDSC (Mettler Toledo DSC1, STAR^{e} System, Switzerland). Thermal transitions were assessed in terms of the T_{g}, measured as the reversible heat flow due to variation in the magnitude of the Cₚ-complex values (ΔCₚ); melting temperature (Tₘ) and crystallization temperature (T_{c}) peaks that were consequences of irreversible heat flow corresponding to the total heat flow. The temperature calibration was accomplished with a melting transition of 6.7 mg indium. The thermal transitions of native CAP were compared to the CAP nanoparticles. Samples of 5 mg were weighed on perforated 40 μL aluminum pans and ramped within a temperature gradient of 150-500° C. under a constant purge of N₂ atmosphere in order to diminish oxidation. The instrument parameter settings employed comprised a sine segment starting at 150° C. with a heating rate of 1° C./min at an amplitude of 0.8° C. and a loop segment incremented at 0.8° C. and ending at 500° C.

### 2.10. In Vitro Assessment of the Matrix Erosion of the Devices

### 2.10.1. The NESD Device

Samples of the biodegradable crosslinked alginate scaffolds were immersed in 100 mL phosphate-buffered saline (PBS) (pH 6.8, 37° C.) and agitated at 20 rpm in a shaking incubator (Labex, Stuart SBS40®, Gauteng, South Africa). At pre-determined time intervals samples were removed, blotted on filter paper and dried to a constant mass at 40° C. in a laboratory oven. Equation 3 was then used to compute the extent of Matrix Erosion after gravimetrical analysis.

$$ME \% = \frac{{M}_{0} - {M}_{t}}{{M}_{0}} \times 100 \qquad Equation 3$$

Where ME % is the extent of scaffold Matrix Erosion, Mₜ is the mass of the scaffold at time t and M₀ is the initial mass of the scaffold.

### 2.10.2. The PNIS Device

Samples were immersed in phosphate buffered saline (PBS) (pH 7.4, 37° C.) and placed into an orbital shaker incubator set to rotate at 20 rpm at 37° C., (Caleva®, Model 7ST, England). Samples were then removed from the PBS solution at specified time intervals, convection dried at 25° C. for 24-48 hours and weighed to gravimetrically determine the degree of matrix erosion. A second set of samples was tested for change in volume after exposure to PBS at predetermined intervals to assess the degree of swelling of the polymeric scaffold.

### 2.10.3. The NBMS Device

Swelling of the NBMS device was determined by immersing a known mass of samples in 10 mL PBS (pH 7.4; 37° C.) in petri dishes (90 mm in diameter) and allowed hydration to take place for 30 minutes. The membranes were allowed to reach the maximum hydration potential and thereafter the swollen mass of the membranes was determined by gravimetric analysis using an electronic analytical mass balance (Mettler Toledo, Inc., Columbus, Ohio, USA) after removing the samples from the PBS solution and blotted with filter paper to adsorb water on the membrane surface. The degree of swelling was calculated as a difference between the mass of the non-hydrated and hydrated membranes (%) employing Equation 4.

$${S}_{D} = \frac{( {W}_{s} - {W}_{i} )}{{W}_{i}} \times 100 \qquad Equation 4$$

Where, S_{D} is the degree of swelling in PBS, and Wᵢ and Wₛ are the masses of the biopolymeric membranes before and after hydration, respectively.

2.11. In Vitro Drug Release from the Devices

### 2.11.1. The NESD Device

In vitro release studies were performed on the DA-loaded nanoparticle formulations and the final NESD utilizing a shaking incubator (Labex, Stuart SBS40®, Gauteng, South Africa) set at 20 rpm. The DA-loaded nanoparticles and NESD was immersed separately in 100 mL phosphate-buffered saline (PBS) (pH 6.8, 37° C.) contained in 150 mL glass jars. At predetermine time intervals 3 mL samples of each release media were removed, filtered through a 0.22 μm Cameo Acetate membrane filter (Millipore Co., Bedford, Mass., USA) and centrifuged at 20,000 rpm [28]. The supernatant was then removed and analyzed by UV spectroscopy at a maximum wavelength of λ₂₈₀ₙₘ for DA content analysis. DA release was quantified using a linear standard curve (R²=0.99). An equal volume of DA-free PBS was replaced into the release media to maintain sink conditions. The Mean Dissolution Time (MDT) values were calculated at 8 hours for each sample using Equation 5. Computing the release data in this manner allowed for the effective model-independent comparison of all formulations in terms of their respective DA release behaviour. All release studies were performed in triplicate.

$$MDT = {\sum }_{i = 1}^{n} {t}_{i} \frac{{M}_{t}}{{M}_{∞}} \qquad Equation 5$$

Where Mₜ is the fraction of dose released in time tᵢ=(t₁+tᵢ₋₁)/2 and M_{∞} corresponds to the loading dose.

### 2.11.2. The PNIS Device

Drug release studies were performed by subjecting scaffolds containing DA-loaded nanoparticles to an orbital shaker incubator, after being immersed in PBS. Samples were taken at predetermined intervals, which were then analysed using Ultra Violet (UV) spectroscopy.

### 2.11.3. The NBMS Device

In vitro release studies were performed in PBS (pH7.4; 37° C.). The biopolymeric membranes were placed in closed 150 mL glass vessels containing 100 mL of the release medium. The membranes were incubated at 37±0.5° C. in an oscillating incubator set at 20 rpm. At predetermined time intervals 5 mL samples of the release medium were removed. Drug-free buffer was replaced into the vessel after sample removal in order to maintain sink conditions. The concentration of MTX was assayed by UV spectroscopy at the maximum drug wavelength λ₃₀₃ₙₘ using a standard calibration curve of known concentrations range from 0.005-0.025 mg/mL with a correlation coefficient R²=0.99.

2.13. In Vivo Analysis of Drug Release from the Devices in a Sprague Dawley Rat Model

Forty five adult male Sprague Dawley rats were used to perform the in viva study. Rats were anaesthetized with a mixture of ketamine (65 mg/kg) and xylazine (7.5 mg/kg) before being placed in a Kopf stereotaxic frame. A straight midline incision (5-10 mm) was made from nasion to occiput. The skin and perisoteum was reflected exposing the dorsal surface of the skull in order to facilitate identification of the cranial sutures and to ensure the skull trephination was made in the frontal hone. A surgical drill was then used to produce a controlled perforation of the skull with an opening of approximately 0.5 mm in diameter followed by sharp incision of the dural lining. The brain parenchyma was then ready for insertion of the NESD. The device was <20% of the rat brain volume (0.000354 cm³ vs. 0.865±0.026 cm³). The wound was sealed with wax and the scalp insertion was closed with a single layer of non-absorbable suture. Temgesic (1 mL) was administered post-operatively for pain relief with a rehydration treatment of 5% glucose in 0.9% saline and a series of behavioral asymmetry tests were performed on the rats to assess any degree of motor dysfunction present. At days 0, 3, 7, 14, 21 and 30 post implantation, the animals were anaesthetized and blood samples (2.5 mL) were collected via cardiac puncture as well as cerebrospinal fluid (CSF) (100-150 μL) through puncturing the cisternal magna and gently withdrawing CSF through a 30-gauge needle and syringe attached to polyethylene tubing. The rats were subsequently euthanized with an overdose of sodium pentobarbitone. All plasma and CSF samples were stored at −80° C. prior to Ultra Performance Liquid Chromatography (UPLC) analysis. A standard curve of drug in fresh plasma was generated from a primary stock aqueous solution of drug (100 mg/mL) and serially diluted to obtain concentrations ranging from 0.0016-30.00 μg/mL. An internal standard was used. Plasma and CSF samples were thawed and acetonitrile (0.4 mL) was added to each sample and centrifuged at 15000 rpm for 10 min. The supernatant was removed and subjected to a generic Oasis® HLB Solid Phase Extraction (SPE) procedure and placed in Waters® certified UPLC vials (1.5 mL). UPLC analysis was performed on a Acquity Ultra Performance Liquid Chromatography system (Waters®, Milford, Mass., USA) coupled with a PDA detector. Separation was achieved on an Acquity® UPLC BEH C₁₈ column (50×2.1 mm, i.d., 1.7 μm particle size) maintained at 25° C. Samples were injected with an injection volume of 5 μL.

2.14. Surgical Implantation of the NBMS Device into the Rat Brain Parenchyma

The rats were anaesthetised with solution of xylazine. Their heads, were shaved and then placed and secured in a stereotaxic frame. A small (0.5-1 cm) para-midline right sided scalp skin incision was made and the scalp periosteum reflected. An electric twist drill was used to make a controlled perforation of the skull approximately 0.5 mm in diameter. The skull opening was followed by sharp incision of the dural lining. The implant was inserted into the brain parenchyma. Post-implantation, the skull defect was sealed with wax and the scalp insertion closed with a single layer of appropriately sized non-absorbable suture. The rats received analgesic medication in the post-operative period. One group of rats was implanted with a placebo device while the other group was implanted with a drug-loaded device.

### 2.15. Histological Evaluation of the NBMS Device

From the brain samples (placebo and drug-loaded implant) recovered at day 30 post implantation, cross-sections were selected from:

A: Mid-section of the anterior half of the cerebrum including the tissue implant on the dorsal aspect of the right cerebral hemisphere.
B: A cross-section from the middle of the posterior half of the cerebrum
C: A cross-section in the middle of the cerebellum
D: a cross-section from the medulla oblongata

From the abovementioned cross-sections tissue blocks specific sections were produced after routine histological processing and stained with haematoxylin and eosin staining in an automated stainer.

### 3. Results and Discussion

### 3.1. Computer-Aided Prototyping for the NESD Design

An output format of serial bitmap images generated via the prototyping technology employed enabled the step-wise 3D volumetric construction of the NESD model. 3D construction was initiated by ascribing an assumed height to each image in order to represent a volume unit or a stacked voxel depicting a prototype model of the NESD described by the grayscale intensity threshold images shown in FIG. 6. Prototyping of the NESD device revealed that the functional properties of the NESD depended on the characteristics of the polymeric materials employed, the processing technique, and the subsequent interaction of fixated CAP nanoparticles within the crosslinked alginate scaffold. The 3D prototype design of the device permitted the porosity, surface area, and surface characteristics to be semi-optimized in the pre-cured and post-cured phases with BaCl₂ for each component of the NESD (FIG. 6a). Fine control of the micro-architectural characteristics influenced the mechanical properties of the scaffold that was significant for nanoparticle fixation and mechano-transduction in order to control the release of DA. A significant advantage of employing prototyping technology to develop the NESD was the elimination of reliance on individual skills that are required for conventional techniques of device fabrication. Commencing with a limited range of fundamental structural units a NESD with precise micro-architectures was designed using prototyping technology with internal channels or cavities resembling the negative image of the final required NESD as depicted in FIGS. 1a, b and c. Visibly, the scaffold models depicted channels that extended through the entirety of the tetragon matrices in both horizontal and vertical axes with consistency in the strand layout after DA-loaded CAP nanoparticle fixation. At the periphery of the matrix, a region of thick and blurred pore deposition was visible after curing the alginate scaffold in BaCl₂ (FIG. 6b). This entire matrix region was approximately 5×3 mm at the edge of the tetragon (FIG. 6 enlarged for clarity). SEM images confirmed the strut and pore widths to be in the range of 100-200 μm. Furthermore, the unconnected pore space, when inspected qualitatively, comprised diminutive cavities within the matrix for controlling the outward diffusion of the DA-loaded CAP nanoparticles from the crosslinked alginate scaffold.

The computational design process revealed that curing of the crosslinked alginate scaffold in BaCl₂ involved the residual crosslinking of open, approachable and chemically reactive molecular functional groups that possessed chemical affinity towards BaCl₂ as the secondary crosslinker and produced an equivalent of edging and interlocking of the matrix surface functional groups with a superiorly compact matrix structure (FIG. 6b). Furthermore DA was not covalently bonded to the CAP with no amide bond formation but interacted ionically via physical associations involving H-bonding and smaller force interactions through the influence of the external crosslinking medium. FIG. 7a represents a structural model of the interactions between H₂O molecules in association with acetate and O₂ functional groups of strongly hydrophilic CAP sites. DA, other ionic species and molecules revealing an interactive model of CAP and DA entrapment constituents are also depicted in FIG. 7b.

FIGS. 8a-e depicts a step-wise single CAP chain structural model under the influence of surrounding interactive forces within the emulsified medium such as solvent molecules at the periphery, PVA as the surfactant and DA. The affinity interactions with explicit lipophilic and hydrophilic orientations towards the formation of a nanoparticle wall are also shown (FIGS. 8f-h). CAP was initially suspended in the binary acetone:methanol solvent system as unorganized random orientations with irregular lipophilic rings (FIG. 8a). The addition of DA and ionic or physical interactions with the hydrophilic functional groups of CAP and free DA molecules resulted in CAP conforming to orientations of the affinity-wise molecular sites in terms of lipophilicity and hydrophilicity of the medium (FIG. 8b). DA also influenced the overall polarity spectrum of the medium. The addition of PVA as a surfactant produced strong molecular associations and crosslinker ions with the subsequent energy supplied via agitation and processing temperatures contributing to surface interactions that produced CAP molecules pivoted toward surface minimization, compactness and orientations of the lipophilic regions (FIG. 8c). The stronger energetic orientations and the presence of PVA as the surfactant tended to sphericalize the CAP strands (FIG. 8d). The CAP strands sphericalized completely to produce nanoparticles under the primary influence of solvent diffusion phenomena and the presence of PVA with the inner core containing DA molecules and lipophilic regions of CAP conforming toward the periphery as the boundary between the outer hydrophilic medium (FIG. 8e). Thus, DA molecules orientated within the hydrophilic voids of the nanoparticles shielded by the lipophilic boundary to form stable CAP nanoparticles.

### 3.2. Computer-Aided Prototyping of the NBMS Device

The immersion precipitation reaction of PLLA and PVA in the presence of the catalyst stannous octoate and triethanolamine (TEA) at 150° C. resulted in the formation of a modified co-polymer with a branched structure. The biopolymeric membranes revealed various consistencies ranging from non-opaque coarse MTX-loaded membranes (FIGS. 9a and c) to opaque smooth membranes (FIGS. 9b and d). The hydrophobic PLLA polymeric chains were conjugated in a graft-like manner onto the hydrophilic PVA backbone via esterification of the hydroxyl groups to form an amphiphilic polymer. The drug (MTX) was subsequently bonded to the PLLA segment as shown in (FIG. 10a). The resultant membrane was shaped through structural polymeric layering to form a porous crystalline hydrogel-based drug delivery matrix (FIG. 10b). The hydration and swelling kinetics of the system were mainly governed by the presence of the hydrophilic PVA backbone that controlled the quantity of water sorption and the extent of swelling of the polymeric matrix. A distinction was the insolubility of the adsorbate in the liquid sub-phase that resulted in the formation of a stable absolute conformation of the biopolymeric membrane that was dependant on the associated surface tension, the surface excess of TEA in comparison to the bulk phase and the concentration of TEA in the bulk phase (FIG. 10c).

Steric hindrance may have shielded MTX binding sites and thus prevented MTX molecules from attaching at every PLLA monomer available along the entire modified polymer backbone accounting for the DEE values attained as discussed later. Thus, MTX binding to the PLLA segment was dependant on the extent of PLLA grafting onto the PVA backbone. To a lesser extent. MTX molecules may also undergo further direct conjugation with free PVA monomers or assemble as freely dispersible entities within the modified polymeric complex. TEA molecules inherently possess dendrimeric properties due to the large number of nitrogen atoms in the entity. A single TEA entity has the capacity of bearing two MTX molecules and may be regarded as a nodal point for drug attachment and drug release. In contrast to the MTX-PLLA-PVA matrices, TEA molecules in the MTX-TEA-PLLA-PVA matrices afforded the system with additional sites for drug attachment (FIG. 11a). The layered structure led to the formation of a multi-layered matrix (FIG. 11b) possessing unique hydration and swelling dynamics and MTX release kinetics. The sparse branching of polymeric chains in the MTX-TEA-PLLA-PVA matrix system afforded greater flexibility due to reduced steric hindrance. The average free volume per molecule available for MTX was increased in contrast to the MTX-PLLA-PVA membrane system.

PLLA co-polymeric conjugate blends with PVA can be modified significantly robust structures by the addition of amphiphilic TEA as a discrete plasticizing and drug binding entity within the matrix. TEA molecules are able to act as stress concentrators, which reduce the overall yield stress of the biopolymeric membrane, allowing plastic deformation, enhanced extensibility and ductile fracture during physicomechanical analysis and drug release studies in PBS (pH 7.4; 37° C.). Crystallized PLLA has significantly reduced impact strength and therefore could be toughened by the addition of TEA as a separate immiscible rubbery phase in conjunction with PVA. Since the biopolymeric membrane is to be used in biomedical applications as a potential drug delivery device, the plasticizer TEA was chosen due to its ability to degrade into substances that are absorbable in the body that are hydrophilic and non-toxic. To develop a mechanistic structural molecular model for the effectual layering of the biopolymeric membrane a mono-layered membranous fusion approach was employed, which has been previously attempted as an effective approach for the formation of supported lipid bi-layered membranes that are able to describe biological cellular membranes with one or more components [81, 82]. The conjugated MTX-TEA-PLLA-PVA-TEA-MTX membrane can be represented by a diverse contoured model in various spatial conformations due to the inherent stereo-electronic factors at the matrix site (FIG. 12a). The formation of a layer is induced by self assembly of conjugated MTX, TEA, PLLA and PVA entities in different ordered orientations, (FIG. 12b). Chirality is able to induce activation at one end of the optically active molecules through linking, binding and association of the conjugated entities that ultimately lead to the formation of a multi-layered membrane structure (FIG. 12c). The process of membrane multi-layering is based primarily on stereochemical factors and the weighted fusion of mono-layers to eventually form a multi-layered structure (FIG. 12d).

Preliminary factors that are required for multi-layered membrane formation is to obtain an even surface following PLLA deposition to ensure the fusion of subsequent layers incorporating MTX molecules. As depicted in the computational structural model generated in FIG. 12 TEA linkage provided an even molecular surface, with a refractivity value of 38.78 A³ for the modeling area (Table 5). The subsequent MTX layer provided a central platform region for structural layering between the isomeric mono-layers (FIG. 12b). Since TEA is amphiphilic the deposition of the tri-branched polyelectrolyte on the membrane surface improved the fusion process due to electrostatic interaction and allowed uniform supported multi-layering to occur. Furthermore, DFT (6-31G) and ab initio computational results of the molecular characteristics employing HyperChem® V7.5 software (Hypercube Inc., Gainesville, Fla., USA) indicated that the addition of TEA dramatically improved the layering effect (Table 5). Approximate surface area values obtained were 0.12 A² and 16.17 A² for the MTX-PLLA-PVA and MTX-OTEA-PLLA-PVA membranes respectively. The acetone:methanol blend was a strong non-solvent for PLLA and PVA. For this approach, compositions with high volume fractions of PLLA at the tri-nodal TEA were in equilibrium with the continuous solvent phase. Due to the strong non-solvent character of the acetone:methanol blend the miscibility gap proved to be sufficient for the immersion precipitation process to occur. Polarizability values of 65.49 A³ and 80.09 A³ were obtained for the MTX-PLLA-PVA and MTX-OTEA-PLLA-PVA membranes respectively. The difference in the polarizability values between the membrane formulations was aligned with the initial difference of 7.54 A³ and 9.39 A³ for native PLLA and PVA respectively (Table 5). Formation of the biopolymeric membranes were a combination of thermodynamic and diffusion kinetic phenomena. In order to induce phase separation by a diffusion-driven process thermodynamic and kinetic conditions were fulfilled. The membrane formation process was governed by diffusion over the interface between the PLLA/PVA solution within the petri dish and the coagulation bath. Although two polymeric components were present in the casting solution only solvent and non-solvent diffused outward. The differences in hydration energy potentials (−11.81 Kcal/mol and 6.86 Kcal/mol for PLLA and PVA respectively) and Log P values (0.47 and 0.12 for PLLA and PVA respectively) conferred the induction of a diffusion flux that was sufficient to compensate for the energy needed to create a new insoluble surface during phase separation resulting in membrane formation at the interface (Table 5). A semi-porous membrane structure was formed and the polymeric solution was in equilibrium with the coagulation bath creating a new structure.

TABLE 5
HyperChem ® V7.5 (Hypercube Inc., Gainesville, FL, USA) computational
molecular attributes at ab initio and Density Functional Theory (DFT) (6-31G)

| | PLLA | PVA | P-P¹ | M-P-P² | T-P-P³ | M-T-P-P | TEA |
|---|---|---|---|---|---|---|---|
| Mass^{a} | 89.0 | — | 232.23 | — | — | — | — |
| Polarizability^{b} | 7.54 | 9.39 | 21.65 | 65.49 | 32.17 | 80.09 | 15.05 |
| Refractivity^{c} | 15.27 | 38.87 | 47.15 | 170.80 | 84.33 | 208.97 | 38.87 |
| Log P | 0.47 | 0.12 | 1.31 | 0.81 | 1.14 | 0.10 | 1.17 |
| Hydration energy^{d} | −11.81 | 6.86 | 7.02 | 0.12 | 1.32 | 16.75 | 13.85 |
| Volume^{e} | 309.6 | A³ | 77.63 | 398.04 | 358.56 | 1807.91 | 166.36 |
| Surface area (approx)^{f} | 38.88 | 159.45 | 393.82 | 16.85 | 160.23 | 823.71 | 242.22 |
| Surface area (grid)^{g} | 228.02 | 178.90 | 422.80 | 245.72 | 243.86 | 980.00 | 143.11 |
| Partial charges^{h} | 0.00 | 0.00 | 0.00 | 0.00 | 0.00 | | |

^{a}amu (atomic mass unit)
^{b,c}and^{e}A³ (Angstrom cube)
^{d}Kcal/mol
^{f}and^{g}A² (Angstrom square)
^{h}and^{e}(net)
¹PLLA-PVA
²MTX-PLLA-PVA
³TEA-PLLA-PVA
4-MTX-TEA-PLLA-PVA

The membranous polymeric scaffold was formed by immersion precipitation, a wet phase separation method based on solvent-non-solvent exchange. Polyvinyl alcohol and polylactic acid 10% w/w, polymer solutions were prepared by dissolving the polymers separately in dimethyl sulphoxide at room temperature 21° C. Polymers were mixed in predetermined ratios and reacted with stannous octoate (esterification reagent) at 150° C. for 60 minutes. The composite polymer was allowed to react with triethanolamine for a further 60 minutes. Polymer samples with folic acid were cast on plastic moulds 15 mm in diameter and immersed in a non-solvent bath composted of 1:1 acetone-methanol mixture for 24 hours. The formed membranes were allowed to dry at room temperature at 21° C. In yet another example, the biopolymeric membrane was prepared by phase separation (immersion precipitation), a wet phase separation method based on solvent-non-solvent exchange. Polymer solutions 10% w/v (PVA and PLA), were prepared by co-dissolving the polymers in dimethyl sulphoxide at room temperature 21° C. Polymers were mixed and further reacted with stannous octoate at 150° C. for 60 minutes. The formed composite polymer solution was then reacted with triethanolamine for 60 minutes. Folic acid 10 mg % was added to the composite polymer solution and cast on glass moulds approximately 15 mm in diameter followed by immersion in a non-solvent bath composted of 1:1 acetone-methanol mixture for 24 hours. The formed membranes were allowed to dry at room temperature at 21° C. The nanoparticles were prepared by double emulsion solvent evaporation technique. The first aqueous solution (W1) was prepared by dissolving folic acid (FA) in a slightly alkaline medium followed by the addition of polysorbate 80 (3% w/v). The organic phase (O) was prepared by co-dissolving the polymers PLA and ES100 in 10 mL mixed solvent system consisting of dichloromethane-isopropyl alcohol in a ratio of 1:1. The aqueous phase (W1) and the organic phase were mixed for 10 min by stirring at room temperature 25° C. to form an emulsion (W1/O). The external aqueous phase (W2) was prepared by dissolving PVA in 200 mL of deionised water. The emulsion (W1/O) was added to the external aqueous phase and emulsification was continued for 30 min using a homogenizer to form a multiple emulsion (W1/O/W2). The nanoparticles were collected by centrifuge, washed two times with deionised water and lyophilised for 24 hours. Tables 6-13 show the experiments used to determination of the upper and lower limits of the independent formulation variables of the membrane and the nanoparticle formulation.

TABLE 6
PVA concentration

| | | Stannous | | Stannous | |
|---|---|---|---|---|---|
| Sample | PLA | Octoate | Triethanolamine | Octoate per | [PVA] |
| Code | (mL) | (mL) | (mL) | 10 mL PVA | (% ^{w}/ᵥ) |
| A-1 | 15 | 0.5 | 0.5 | 1 | 10 |
| A-2 | 15 | 0.5 | 0.5 | 1 | 15 |
| A-3 | 15 | 0.5 | 0.5 | 1 | 20 |
| A-4 | 15 | 0.5 | 0.5 | 1 | 25 |

TABLE 7
PVA volume ratios

| | | Stannous | | Stannous | PVA |
|---|---|---|---|---|---|
| Sample | PLA | Octoate | Triethanolamine | Octoate per | volume |
| Code | (mL) | (mL) | (mL) | 10 mL PVA | ratio |
| B-1 | 15 | 0.5 | 0.5 | 1 | 25 |
| B-2 | 15 | 0.5 | 0.5 | 1 | 35 |
| B-3 | 15 | 0.5 | 0.5 | 1 | 45 |
| B-4 | 15 | 0.5 | 0.5 | 1 | 55 |

TABLE 8
Triethanolamine concentration

| | | Stannous | | PVA | |
|---|---|---|---|---|---|
| Sample | PLA | Octoate per | [PVA] | volume ratio | Triethanolamine |
| Code | (mL) | 10 mL PVA | (% ^{w}/ᵥ) | (mL) | (mL) |
| C-1 | 15 | 1 | 10 | 20 | 0.5 |
| C-2 | 15 | 1 | 10 | 20 | 1.5 |
| C-3 | 15 | 1 | 10 | 20 | 2.5 |
| C-4 | 15 | 1 | 10 | 20 | 5.0 |

TABLE 9
Drug loading capacity

| | | Stannous | | PVA | |
|---|---|---|---|---|---|
| | | octoate | PVA | volume | |
| Sample | PLA | per 10 mL | concentration | ratio | Triethanolamine |
| code | (mL) | PVA | ^{w}/ᵥ (%) | (mL) | (mL) |
| D-1 | 15 | 1 | 20 | 0.5 | 15 |
| D-2 | 15 | 1 | 20 | 0.5 | 30 |
| D-3 | 15 | 1 | 20 | 0.5 | 30 |

TABLE 10
PLA concentration

| | | | Volume of | |
|---|---|---|---|---|
| Formulation | PLA | ES100 | aqueous | Concentration of the |
| code | (mg/mL) | (g/mL) | phase (mL) | external phase |
| PLAES3025 | 3.0 | 2.5 | 2 | 0.5 |
| PLAES2525 | 2.5 | 2.5 | 2 | 0.5 |
| PLAES1525 | 1.5 | 2.5 | 2 | 0.5 |

TABLE 11
ES 100 concentration

| | | | Volume of | Concentration |
|---|---|---|---|---|
| Formulation | PLA | ES 100 | aqueous | of the external |
| code | (mg/mL) | (mg/mL) | phase | phase (mg/mL) |
| PLAES3050 | 3.0 | 5.0 | 2 | 0.5 |
| PLAES3030 | 3.0 | 3.0 | 2 | 0.5 |
| PLAES3015 | 3.0 | 1.5 | 2 | 0.5 |

TABLE 12
Volume of aqueous phase

| | | | Volume of | Concentration of the |
|---|---|---|---|---|
| Formulation | PLA | ES 100 | aqueous | external phase |
| code | (mg/mL) | (mg/mL) | phase | (mg/mL) |
| PLAES30251 | 3.0 | 2.5 | 1 | 0.5 |
| PLAES30252 | 3.0 | 2.5 | 2 | 0.5 |
| PLAES30253 | 3.0 | 2.5 | 3 | 0.5 |

TABLE 13
External phase concentration

| | | | Volume of | Concentration of the |
|---|---|---|---|---|
| Formulation | PLA | ES100 | aqueous | external phase |
| code | (mg/mL) | (mg/mL) | phase | (mg/mL) |
| PLAES30254 | 3.0 | 2.5 | 2 | 0.25 |
| PLAES30255 | 3.0 | 2.5 | 2 | 0.5 |
| PLAES30256 | 3.0 | 2.3 | 2 | 1.0 |

### 3.3. Physicomechanical Analysis of the Devices

### 3.3.1. The PNIS Device

Polymer scaffolds displayed an average resilience of 4.92%, confirming the presence of uniformly sized pores within the polymer matrix, which may serve to reduce matrix erosion, enabling prolonged drug released once implanted into the intracranial cavity of the brain. Scaffold hardness was calculated to 3.45 Nm, which is expected to decrease with prolonged exposed to PBS (FIGS. 13a and b).

### 3.3.2. The NBMS Device

The physicomechanical strength of the biopolymeric membranes depended profoundly upon the polymer linkages. Dissimilar and unique degrees of extensibility were observed for the various biopolymeric membranes (FIGS. 14a and b). Extensibility can be defined as the degree to which a material can be extended/stretched prior to fracture and is related to the elasticity of the material. The inclusion of TEA in the membrane formulation resulted in a significant transition of the physicomechanical properties of the membranes. The MTX-TEA-PLLA-PVA membrane was superiorly robust with a considerably higher extensibility compared to the MTX-PLLA-PVA membrane as a greater force of extension and larger fracture distance was required (FIG. 14a). When the elastic limit of the MTX-TEA-PLLA-PVA membranes was reached a greater resistance to structural deformation was noted (region p in FIG. 14b). The variation in the textural properties may be related to the computational structural models that proposed the mechanism of membrane formation for the two formulations (MTX-TEA-PLLA-PVA and MTX-PLLA-PVA) leads to the different layered structural architectures.

Textural profile analysis revealed that the biopolymeric membrane was significantly toughened by the introduction of TEA as a discrete rubbery phase within the co-polymer matrix. The MTX-TEA-PLLA-PVA biopolymeric membrane system was tougher (F=89N) and considerably more extensible (D=8.79 mm) compared to MTX-PLLA-PVA (F=35N, D=3.7 mm) membranes since a greater force of extension and fracture distance was required. The MTX-TEA-PLLA-PVA membrane showed superior resistance to structural deformation. TEA molecules acted as a stress concentrator that reduced the overall yield stress of the membrane, allowing plastic deformation and ductile fracture to occur prior to membrane fracture (FIG. 14b; region p). The grafted TEA molecules lowered the force required for fracture and therefore considerably increased the quantity of dissipated energy during fracture. PLLA quenched from the melt or non-crystallizable L- and D-lactide has a low impact strength. Thus the reduced strain to break during bi-axial extensibility testing was sensitive to minute surface imperfections. PLLA was therefore significantly toughened by blending with TEA as a separate, immiscible rubbery phase. The strength of the MTX-PLLA interface bond was a significant parameter for not only toughening of the biopolymeric membrane but also MIX entrapment and subsequent release. The strength of this interface was modified by the use of TEA as a compatibilizer, graft and block co-polymer.

### 3.4. Morphological Characterization of the Devices

### 3.4.1. The NESD Device

The crosslinked alginate scaffold displayed an average pore size of 100-400 μm with a wall thickness calculated at an average of 10±1.04 μm. The pores allowed for the efficient diffusion and release of CAP nanoparticles within the crosslinked scaffold micro-architecture. Scaffolds that were not subjected to post-curing in a secondary crosslinking BaCl₂ solution revealed a “tissue-like” appearance (FIG. 15a) in comparison to the evenly distributed porous crystalline yet compact appearance of post-cured scaffolds (FIG. 15b).

SEM images of the CAP nanoparticles depicted exemplary particles in both DA-free and DA-loaded states (FIGS. 15c and d). The spherical particles were uniform in size with a distinct non-aggregated architecture. TEM images of DA-free particles revealed opaque structures with variations in size (FIG. 15e). DA-loaded CAP nanoparticles were slightly transparent with a degree of transient aggregation (FIG. 15f). Overall both DA-free and DA-loaded CAP nanoparticles displayed patent surface morphologies.

### 3.4.2. The PNIS Device

TEM images, FIGS. 16a and b, revealed the presence of particles ranging between 200-700 nm as well tubes ranging between 500-900 nm with particles present within the tubes ranging between 50-200 nm.

SEM images revealed highly porous scaffolds, with small uniform pores present within the scaffold matrix, which may aid in the even dispersion of AZT-loaded nanoparticles, serving to enhance AZT delivery (FIG. 17).

### 3.4.3. The NBMS Device

High magnification SEM revealed distinct continuous layers of the biopolymeric membrane with macro-porous mosaic morphologies (FIG. 18a). The high level of crystallinity is evident from the randomly shaped macro-pores with very sharp distinct borders (FIG. 18b). Following the immersion step, the top surface of the membrane (FIG. 18c) is formed spontaneously by the non-solvent-solvent diffusion process which occurs immediately at the polymer-non-solvent interphase. The bottom surface of the membrane (FIG. 18d) is formed gradually as the non-solvent penetrates into deeper membrane depths and this result in the formation of more consistent mosaic morphology.

The formation of pores within the membrane depended on the sequence of the phase transition events in the immersion precipitation process [83, 84]. Numerous parameters, such as the compositions of the polymeric solution the precipitation bath and the temperature during preparation influenced the morphology and surface area of the formed membranes. Since membrane preparation is a non-equilibrium process, this clearly implied that the change in membrane structure was attributed to the arrangement of PLLA and PVA chains during membrane formation. A high polymer concentration region was formed at the interface between the polymer solution and the coagulation bath. High polymer concentration at this interface acted as a diffusion barrier to mass transport. Phase separation at this stage will have no influence on the asymmetry of the process, which explains the symmetry in structure of the biopolymeric membranes. Fine particles were noted under high SEM magnification (FIGS. 18a and b). These particles may have been generated due to crystallization of polymer during the membrane formation process [70, 71].

### 3.5. Polymeric Molecular Structure Variation Analysis of the NESD and PNIS Devices

FTIR spectra for DA-free nanoparticles revealed a broad stretch band (1070-1242 cm⁻¹ and 3200-3600 cm⁻¹) representing OH⁻ groups and a stretch band (2926 cm⁻¹) indicating alkane moieties while a band at 1731 cm⁻¹ revealed the presence of —C═O within the CAP nanoparticle structure. The interpretation demonstrates the definitive presence of impervious CAP in DA-free nanoparticles. The spectra for DA-loaded CAP nanoparticles also confirmed the presence of CAP (bands at 1070, 1242 and 2926 cm⁻¹) while the possible interaction of CAP OH⁻ functional groups with the —NH₂ group of DA may have resulted in the formation of nitro compounds (1390 cm¹). The interaction between the H⁺ of the NH₂ group on DA and the O⁻ atom of the OH⁻ group on CAP may have culminated in the proposed physical interactions of the two compounds retarding DA release as predicted initially via the prototyping technology employed. FTIR images of the PNIS nanoparticles (FIG. 19), indicated a change in the surface morphology of both the nanoparticles and the scaffold due to surface interactions occurring during the preparation process. However, basic polymeric structure of the parent compounds was maintained.

3.6. Assessment of the Size and Stability of the Nanoparticles within the Devices

### 3.6.1. The NESD Device

A nanoparticle z-average size of 1654 nm and 241 nm was recorded for DA-free and DA-loaded CAP nanoparticles, respectively. The result was atypical as it was expected that the DA-free CAP nanoparticles would have a smaller size in comparison to the DA-loaded particles due to the absence of drug. However, the zeta potential of DA-loaded CAP nanoparticles displayed increased stability in comparison to the DA-free particles. DA-free particles therefore aggregated more easily, contributing to the relative increase in size. A polydispersity index (PdI) value of 0.030 was calculated for the DA-loaded CAP nanoparticles indicating minimal variation in particle size (165-174 nm) and highlighting the uniformity of particle size in the formulation. Zeta potential values of −23.1 mV and −35.2 mV were recorded for DA-free and DA-loaded CAP nanoparticles respectively. While this result was indicative of the desirable lack of particle agglomeration in both DA-free and DA-loaded particles, it also revealed that the DA-loaded CAP nanoparticles displayed superior stability in comparison to DA-free particles. FIG. 5 depicts typical size and zeta potential intensity profiles generated (FIG. 20).

### 3.6.2. The PNIS Device

Particle size distribution studies revealed an average size distribution of 576.1 d·nm for AZT-loaded nanoparticles, and 602.4 d·nm for drug-free nanoparticles. Wider peaks were obtained as seen in FIG. 21a. This is due to the tendency of nanoparticles to agglomerate. The average zeta potential of AZT-loaded nanoparticles was −0.174 and that of drug-free nanoparticles was −6.39. Inclusion of a 1% w/v PVA solution in the formulation enhanced the average size distribution and zeta potential to 33.21 d·nm for the Z-average and −2.37 for Z-potential. This may be due to PVA conferring surfactant properties and thus reducing agglomeration.

### 3.6.3. The NBMS Device

Nanoparticles with the size distribution within a range of 160-800 nm were formed by preliminary experimental design. PLA seemed to be the major variable that determined the size of the nanoparticles. High zeta potential measurements (−20 mv) were obtained at 1% PVA external phase indicating good particle stability. The PVA/ES100 nanoparticles are suitable for embedding into PLA/PVA biopolymeric membrane system for sustained modulated delivery of chemotherapeutic agents. FIGS. 22a-f depicts the size and zeta potential distribution profiles of the various nanoparticle formulations.

The size of the nanoparticles increased as the concentration of PLA increased in the formulation. An increase in the amount of Eudragit ES100 also resulted in an increase in the size of the nanoparticle although at a much more less extent compared to PLA. The zeta potential measurement could only be improved by increasing the concentration of the external aqueous phase from 0.25-1.0%.

### 3.7. Componential Thermal Analysis on the NESD

TMDSC profiles portrayed the paradigms of the thermal behavior in the three componential elements of the NESD that included the CAP nanoparticles, the crosslinked alginate scaffold and the NESD as shown in FIGS. 23a, b and c. The changes in T_{g}, Tₘ and T_{c} that occurred upon the formation of DA-loaded CAP nanoparticles, the crosslinked alginate scaffold and the assimilated NESD when compared to native CAP employed for nanoparticle fabrication is depicted in FIGS. 23a-c.

All components presented with triple exothermic peaks depicting a coincidental similarity in crystallization behaviors (T_{c}) (FIGS. 23a, b and c). The similarity in thermal behavior between the crosslinked alginate scaffolds and NESD portrayed a direct indication of the high degree of crystallinity imparted by the secondary crosslinker BaCl₂ that was employed as a curing step for scaffold formation. Noteworthy was the significantly large variation in T_{g} and Tₘ between the native CAP (T_{g}=160-170° C.; Tₘ=192° C.) and the DA-loaded CAP nanoparticles (T_{g}=260° C.; Tₘ=268° C.). The apparent shifts in T_{g} and Tₘ elucidated a possible interfacing between CAP and DA molecules that contributed to the formation of physical interactions culminating into the thermal behaviour observed. The large positive shifts in thermal events may have also influenced the release of DA from the CAP nanoparticles as supported by the initial prototyping technology employed and DA release profiles discussed later on. The presence of transient melting endothermic peaks and further shifts in T_{g} observed on the TMDSC signals of the NESD samples clearly reflected the effect of altered thermal properties produced by initial crosslinking between [HOCH₂(CHOH)₄COO]₂Ca and alginate and further the dispersion of DA-loaded CAP nanoparticles within the BaCl₂ solution as a post-curing process. The altered thermal behaviour influenced the physicomechanical behaviour as supported by the earlier morphological, textural profile and FTIR analysis. Overall, the thermal behavior observed may be due to variation in the ΔH involved, ability to attain near-equilibrium conditions during measurement, and the rapid rate of change in molecular rearrangement compared to the ΔT. These pertinent intermolecular interactions, which resulted in the observed thermal transitions (FIGS. 23a, b and c), may have also contributed substantially to the superior control of DA released from the NESD.

### 3.8. Drug Entrapment Efficiency Studies

### 3.8.1. The NESD Device

An average drug entrapment efficiency (DEE) value of 63±0.35% was computed for the DA-loaded nanoparticles. This was considerably high for a nanoparticle formulation (which exhibits a larger surface area) and in particular for a highly water-soluble molecule such as DA. DA had a greater affinity for the aqueous phase of the emulsion therefore increasing the DEE value.

### 3.8.2. The NBMS Device

Relatively high MTX-loading capacities were achieved for both membrane formulations (FIG. 24a). A significant difference in the swelling potential of the two formulations was also observed (FIG. 24b).

Biopolymeric membranes that are formed by immersion precipitation of polymeric solutions in coagulation baths with a high solvent concentration, variations in the casting solution and the coagulation bath may have significant consequences on the DEE and swelling behavior of the membranes. The MTX-TEA-PLLA-PVA membranes showed a higher degree of swelling (53±0.5%) compared to the MTX-PLLA-PVA membranes (28±0.5%) (FIG. 24b). This was due to the ability of the MTX-TEA-PLLA-PVA system to imbibe a larger quantity of water molecules due to its multi-layered conformational structure. The high MTX entrapment values indicated that the energy gain of de-mixing MTX was probably larger than the energy needed to form a new interface for membrane formation. The extent of phase separation and further MTX entrapment can be enhanced by varying the molecular mass of the polymers, adjusting the blending procedures, and annealing the blended materials.

### 3.9. Statistical Response Surface Analysis

### 3.9.1 Analysis of Matrix Resilience of the NESD Device

An increased scaffold Matrix Resilience (MR) was observed at higher alginate (2-3% w/v) and [HOCH₂(CHOH)₄COO]₂Ca concentrations (0.3-0.4% w/v) (FIG. 7a). This was expected as at higher alginate concentrations an advanced degree of crosslinking occurs producing a superiorly robust and interconnected polymeric networked structure with the increased availability of [HOCH₂(CHOH)₄COO]₂Ca. Higher processing temperatures (60-70° C.) and lower concentrations of alginate (1% w/v) also provided a desirable MR value (FIG. 25b). This was attributed to the enhanced molecular mobility of alginate polymeric chains at higher temperatures that induced participation in the crosslinking reaction resulting in the preferred micromechanical behavior. The concentration of [HOCH₂(CHOH)₄COO]₂Ca had the most significant effect in terms of achieving superior MR (p≦0.05) with increased concentrations providing higher MR values, while the processing temperature displayed the most significant role in matrix design (p≦0.05). A processing temperature of 50° C. also provided desirable MR values. However this was not relevant for post-curing times of 60 minutes.

### 3.9.2. Analysis of Mean Dissolution Time of the DA-Loaded Cap Nanoparticles

The altering DA release profiles for the respective CAP nanoparticulate formulations are represented in FIG. 25, signifying the ability to flexibly modulate the release of DA from the nanostructures. A physical incompatibility described by discontinuous aggregation and subsequent clustering between the predominant polymers CAP and PVA was noted. An increase in CAP concentration (0.75-1% w/v) and a decrease PVA concentration (0.5% w/v) led to a higher MDT value and vice versa. The concentration of PVA had the greatest influence on the MDT value where concentrations that were either < or > 1.25% w/v had a positive effect on MDT. This showed that the increase in PVA concentration (1.5-2% w/v) was able to control and limit DA release. Lower stirring speeds (300 rpm) also displayed higher MDT values (41.75) presumably due to the efficient entrapment of DA at lower agitation during processing. Furthermore a higher MDT value was most significantly contributed by a decrease in stirring rate and time. Thus overall, a decreased stirring speed allowed for the adequate homogenization of the formulation components prior to particle micronization and significantly increased DA entrapment and the ability to control the release of DA from the CAP nanoparticles.

### 3.9.3. Analysis of Particle Size of the DA-Loaded Cap Nanoparticles

FIG. 25d revealed that an increase in stirring speed (300-700 rpm) had an unfavorable effect on particle size with particles produced within a larger size range of 150-300 nm. A prolonged emulsification phase of between 150-180 min coupled with a desirable lower stirring speed resulted in the formation of dispersed non-aggregated particles with a reduced particle size of maximum 200 nm (FIG. 25d). An interesting observation was that a decrease in CAP concentration (0.5% w/v) resulting in increased particle sizes ranging from 200-225 nm. DEE results obtained from the experimental design template (Table 4) demonstrated that a significantly lower DEE was achieved with an increase in CAP concentration which may have resulted in decreased particle sizes. The concentration of PVA was also influential in terms of particle size, with particle sizes increasing with an increase in PVA concentration coupled with higher stirring speeds (p≦0.05). The velocity at which PVA Was agitated was sufficient to ensure homogeneity and the impartation of surfactant properties to the formulation thereby reducing the risk of particle attraction that could produce unfavorably larger particle sizes.

### 3.94. Analysis of Zeta Potential of the DA-Loaded Cap Nanoparticles

An increase in PVA concentration (1.5-2% w/v) provided desirable zeta potential values ranging between −30 mV to −35 mV (FIG. 25e). This was expected as PVA was added due to its ability to act as an absorptive surfactant that decreased the interfacial tension and thus imparted stability to the formulation (p≦0.05). FIG. 12b showed that an increased in stirring speed (500-700 rpm) and a reduced emulsifying time of 30 min also resulted in desirable zeta potential values ranging between −30 mV to −35 mV. The higher agitation velocity prevented the particles from aggregating and eliminated the possibility of sedimentation or caking of the nanoparticles. A distinct relationship between lower CAP concentrations and suitable zeta potential values was noted in consideration of the physical incompatibility between CAP and PVA.

### 3.10. Analysis of Matrix Erosion/Swelling of the Devices

### 3.10.1. The NESD Device

An increase in alginate concentration (2-3% w/v) resulted in a reduced scaffold Matrix Erosion (ME) (FIG. 25c) as a result of a superiorly compact scaffold produced from a precursor solution of increased viscosity. Furthermore an increase in [HOCH₂(CHOH)₄COO]₂Ca resulted in a greater degree of crosslinking thereby increasing the scaffold rigidity and retarding ME. An increase in processing temperature (60-70° C.) and post-curing time (60-90 min) retarded the ME. Higher temperatures enhanced the aqueous solubility of [HOCH₂(CHOH)₄COO]₂Ca and a prolonged post-curing period allowed for optimal crosslinking and subsequently controlling the rate and extent of ME. Furthermore an increase in [HOCH₂(CHOH)₄COO]₂Ca concentration facilitated ME (p≦0.05). This was unexpected and may have resulted from an excess in free [HOCH₂(CHOH)₄COO]₂Ca ions present at the saturation point that initiated auto-catalysis and the rapid dissolution of the highly water soluble crosslinker from the scaffold thus increasing the scaffold ME. A higher processing temperature coupled with a decreased alginate concentration also retarded the (p≦0.05). At higher processing temperatures further uniformity and efficiency in the distribution of alginate became apparent within the scaffold matrix thereby contributing to the superiorly controlled ME dynamics.

### 3.10.2. The PNIS Device

Nanoparticles and polymer scaffold were found to be stable upon exposure to PBS, pH 7.4. Matrix erosion studies performed on the polymer scaffold indicated an average percentage mass loss of 28% over 10 hours (FIG. 26 and Table 14). Scaffolds were found to swell considerable, with an average percentage change in volume of 65% in the first hour, which then decreased to 20% after 5 hours and increased to 120% after 25 hours (FIG. 26).

TABLE 14
Mass loss (%) of PCL-ECL scaffolds

| Time | Initial Mass | Final Mass | % Mass Loss |
|---|---|---|---|
| 1 hour | 631.2 | 555.6 | 11.977 |
| 3 hours | 1257 | 1072.7 | 14.662 |
| 12 hours | 1043.4 | 810.3 | 22.34 |
| 24 hours | 669.2 | 516.2 | 22.86 |
| day 3 | 698.5 | 485 | 30.57 |
| day 5 | 777.9 | 567.7 | 27.02 |
| day 7 | 840.1 | 603.5 | 28.16 |
| day 9 | 1183.3 | 858 | 27.49 |
| day 11 | 868.5 | 610.7 | 29.68 |
| day 13 | 581.6 | 416 | 28.47 |
| day 15 | 1184.2 | 867.1 | 26.78 |

The main effects plots showed that an increase in [crosslinker] promoted mass loss (p=0.098) (FIG. 27b). This was unexpected however this could have resulted from an excess in free ca-gluconate in the formulation due to its inability to crosslink with alginate (as the process had reached saturation) thereby resulted in the rapid dissolution of the highly water soluble crosslinker from the scaffold, decreasing scaffold mass. A higher temperature coupled with decreased [alginate] gave rise to reduced mass loss (FIG. 28b) shown in the interaction plots. At increased temperature, there is more uniform and efficient distribution of the alginate (especially at lower concentrations) throughout the formulation thereby displaying more desirable erosion %. Higher temperatures result in more efficient annealing of the polymer which ultimately improves mechanical integrity of matrix with resultant decreased erosion.

### The Main and Interaction Effects on the Responses: Resilience and Erosion of Ba-Alginate Scaffolds

### The Main and Interaction Effects on the Responses: MDT, Particle Size and Zeta Potential of the DA-Loaded Nanoparticles

### Analysis of the Box-Behnken Design Employed for Formulation Optimization

Ba-alginate scaffolds: Residual analysis for resilience (FIG. 31a) and erosion (FIG. 31b) showed the casual distribution of data. The normal plot of residuals displayed slight curvatures of the lines which occurred due to the decreased observation points (less than 50) however the plot still showed normal distribution of the data. The residuals versus fitted plot showed randomly scattered data points around the horizontal line (residual=0), with some fanning in FIG. 31a indicative of a degree of non-constant variance, and were within 3 standard deviations of the mean, i.e., zero. The histogram supported that the residuals have a normal distribution with zero mean and constant variance. The residuals versus the order of the data was used to identify non-random error, the plot showed a both a positive (clustering of formulations 4-12) and a negative correlation indicated by rapid changes in the signs (−/+) of the consecutive residuals thereafter.

Residual analysis for MDT (FIG. 32a), particle size (FIG. 32b) and zeta potential (FIG. 32c) showed the casual distribution of data. The normal plot of residuals formed a straight line showing normal distribution. The residuals versus fitted plot showed a random pattern of residuals on either side of 0 with no identifiable patterns in the plot thereby indicative of a random scatter and no trends. The histogram supported that the residuals have a normal distribution with zero mean and some constant variance. The residuals versus the order of the data was used to identify ant non-random error, the plot showed a negative correlation is indicated by rapid changes in the signs (−/+) of the consecutive residuals.

### 3.11. Constrained Optimization of the Devices

### 3.11.1. The NESD Device

Optimization of the NESD was performed employing Minitab® V15 statistical software (Minitab Inc., PA, USA) to determine the optimum level for each variable for both the crosslinked alginate scaffold and DA-loaded CAP nanoparticles. The optimization process resulted in the attainment of formulations with a considerably low desirability value for all three outcomes. Thus a selective approach based on the most influential desired outcome was used. The Matrix Resilience and Matrix Erosion were the most significant characteristics optimized for the crosslinked alginate scaffold. The MDT value for the CAP nanoparticles was further controlled by the incorporation of the DA-loaded CAP nanoparticles within the crosslinked alginate scaffold and the zeta potential value was alterable via uniform distribution throughout the scaffold during formulation. Therefore, the CAP nanoparticles having the smallest particle size with high desirability (>99%) was selected as the optimal nanoparticle formulation. Residual analysis of the scaffold Matrix Resilience, Matrix Erosion, the MDT values of the nanoparticle formulations; particle size and zeta potential showed the random distribution of data. Normal residual plots displayed insignificant profile curvature due to a reduction in observation points (<50) however maintained normality for the scaffold optimization. The residual plots for CAP nanoparticle optimization were distinctly linear with normality. Residual versus fitted plots displayed data randomness along the baseline residual value of 0 within three standard deviations of the mean. Furthermore, no expression of blueprinting was indicative of a trendless circumstance. This was supported by histograms depicting the residuals having a normal distribution with a zero mean and a constant variance. Non-random error identification plots revealed typical positive (clustering of formulations 4-12) and negative correlation indicated by rapid changes in the signs (−/+) of the consecutive residuals.

### 3.11.2. The PNIS Device

Optimization was performed employing statistical software (Minitab®, V14, Minitab, USA) to determine the optimum level for each variable for both Ba-alginate scaffolds and CAP DA-loaded nanoparticles (FIGS. 33 and 34). The optimization process resulted in the attainment of various formulations with a significantly low desirability for all three outcomes therefore a selection of the most influential desired outcome was necessary to the detriment of the other two outcomes. MDT of the nanoparticles could be controlled further by the incorporation of nanoparticles into the scaffold while zeta potential could be altered by uniform distribution throughout the scaffold during formulation. Therefore, the nanoparticle formulation displaying the smallest particle size with high desirability (>99%) was selected as the optimal formulation (Table 15). Resilience and erosion were the most important and essential characteristics for the scaffold and so a scaffold formulation displaying both characteristics at optimal level was selected.

TABLE 15
Optimized responses for scaffold

| Measured | | | | |
|---|---|---|---|---|
| Response | Formulation | Predicted | Experimental | Desirability |
| Resilience (%) | 1 | 92.6650 | 82.455 | 88.982 |
| Erosion (%) | 1 | 21.9500 | 18.23 | 83.052 |

### 3.12. Desirability Analysis for the Measured Responses for the Devices

### 3.12.1. The NESD Device

With reference to the optimized crosslinked alginate scaffold, the Matrix Resilience of the experimental formulation (82.46%) displayed favorability to the fitted formulation (88.98%). While the experimental formulation had a slightly lower Matrix Resilience than the fitted, this was counteracted by the Matrix Erosion which was lower than predicted (only 18.23% after 7 days) (Table 16). The optimized NESD formulation proved to have the desired characteristics of increased Matrix Resilience and a decreased Matrix Erosion. For the optimized DA-loaded CAP nanoparticles, the MDT value desirability of 94.41% was the most promising outcome and therefore DA release from the CAP nanoparticles were controlled and sustained for the period of time desired. With reference to the particle size (possessing a statistical desirability of 76.15%); while the value of 197 nm (Table 16) was not ideal for the optimally specified system, it was within the limits set for medicinal nano-therapeutic systems of <200 nm [29]. The desirability value of 76.68% obtained for the zeta potential optimization signified that it differed substantially from the fitted value with a superior value in terms of stability of −34.00 mV for the optimized system. Overall, the optimized system displayed the desirable DA release, size and stability required for utilization as an intracranial device for the prolonged and controlled delivery of DA to the brain tissue.

TABLE 16
Optimized responses for nanoparticles

| Measured | | | Experi- | Desir- |
|---|---|---|---|---|
| Response | Formulation | Predicted | mental | ability |
| Zeta Potential (mV) | 1 | −26.072 | −34.000 | 76.682 |
| Size (nm) | 1 | 150.175 | 197.200 | 76.154 |
| MDT | 1 | 43.505 | 40.956 | 94.414 |

### 3.12.2. The PNIS Device

Ba-alginate Scaffold: The resilience of the experimental formulation was in fair agreement with the predicted value demonstrating the reliability of the optimization procedure (Tables 4 and 5). While the experimental formulation showed slightly lower resilience than predicted, this was counteracted as the erosion was lower than predicted (only 18.23% post one week). The optimized formulation proved have the desired characteristics of increased resilience and decreased erosion.

CAP DA-loaded Nanoparticles: The value for MDT desirability (94.414%) was the most promising outcome and therefore DA release of the nanoparticle system would be controlled and sustained for the period of time desired. As for the particle size, while the value of 197.2 nm for the optimum formulation (FIG. 17a) was not ideal it was within the limits set for medicinal nano-therapeutic systems (<200 nm). Furthermore, the particles do not need to cross through the Blood-Brain Barrier and thus the size may exceed 100 nm. The zeta potential desirability (76.68%) was away from the predicted value however was actually superior (FIG. 17b) than the predicted optimized system in terms of stability. Overall, the optimized system displayed the desirable drug release, size and stability required for the type of drug delivery system developed.

3.13. In Vitro Drug Release Studies from the Devices

### 3.13.1. The Nesd Device

The release of DA from the NESD (FIG. 35) displayed an initial lag phase compared to the CAP nanoparticles which were not configured within the crosslinked alginate scaffold. The mechanically patent and interconnected crosslinked alginate scaffold aided in reducing the initial burst effect of DA. The controlled migration of the CAP nanoparticles from the scaffold to the diffusional environment ultimately served to modulate the release of DA at the site of implantation.

### 3.13.2. The PNIS Device

Drug release studies indicated first-order kinetics, whereby approximately 100% entrapped AZT was released from the nanoparticles within 4 hours. Incorporation of nanoparticles into the CMC-ECL-PEO polymeric scaffold significantly retarded drug release (after 4 hours 3.43% drug was release). Zero-order drug release was observed (FIG. 36).

Nancparticles dispersed within the PCL-ECL scaffold displayed a more significant decrease in drug release, with drug release as low as 2.09% being obtained after 35 days.

### 3.13.3. The NBMS Device

The release of MTX from the biopolymeric system followed tri-phasic kinetics. An initial burst in MTX release was observed due to unbound MTX molecules entrapped within the polymer matrix. The initial burst phase of MTX release (Phase I) (unbounded MTX) was followed by steady state kinetics (Phase II) (TEA-bound MTX) presumably due to the gradual hydration and swelling of the biopolymeric membrane. A final controlled up-curving MTX release phase (Phase III) was observed due to a combination of surface and bulk erosion of the membrane (FIGS. 37a and b). The quantity of MTX released from the MTX-PLLA-PVA system (FIG. 9a) at a particular time-point was greater than that released by MTX-TEA-PLLA-PVA system (FIG. 37b) since more energy was required to break the TEA-MTX bond by hydrolytic cleavage. Therefore confirming the function of TEA to act as a drug binding motif that was able to modulate MTX release.

The biopolymeric membrane formulations, MTX-PLLA-PVA and MTX-TEA-PLLA-PVA, are amphiphilic structures with a thin planar geometry. The amphiphilic character is attributed to the hydrophobic characteristics of the PLLA branches and the hydrophilic characteristics of the PVA backbone. The degradation kinetics of the membranes will therefore deviate from those of a hydrophobic polymeric networks fabricated from native PLLA or PVA based hydrophilic hydrogels. The limited water sorption capabilities of PLLA are improved by conjugation onto the PVA backbone and the resultant modified polymer will thus possess the favourable properties of hydrogels. The computational structural molecular models depicted evidence of in situ MTX loading and therefore the biopolymeric membranes are highly likely to adopt a chemically-controlled mechanism of MTX release. However MTX release profiles from the two formulations (with and without TEA) differed owing to the presence of the MTX-binding motif TEA in the MTX-TEA-PLLA-PVA membrane. A purely kinetic-controlled release mechanism which occurs via bond cleavage and mediated by surface erosion may be responsible for MTX release modulation. The hydrolytic cleavage of the MTX-polymer covalent bond in the MTX-PLLA-PVA membrane is the rate limiting step with regard to MTX release. However, a different situation prevails with the MTX-TEA-PLLA-PVA membranes where TEA is tethered to MTX molecules, the kinetics and thermodynamics of which will determine the release kinetics of MTX from the membrane. The structural integrity of the membranes will be maintained since they would obey surface eroding phenomena.

3.14. In Vivo Analysis of DA Release from the NESD in the Sprague Dawley Rat Model

The generic SPE procedure selected in order to isolate DA from the plasma and CSF samples was suitable for retaining the polar DA compound. Serial dilutions of methanol solutions ranging from 5-100% v/v with either the addition of acetic acid or sodium hydroxide were employed in the SPE procedure. It was noted that during the acidic phase (CH₃COOH) higher integral UPLC peaks and extraction yields were obtained as compared to the basic phase (NaOH), in particular, at 70% v/v methanol with 2% v/v acetic acid. An additional wash-step of 45% v/v methanol produced even larger recoveries and level chromatographic baselines. The extraction recoveries ranged from 95.89-101.02%, while the precision values ranged from 3.5-11.7% over three concentrations evaluated over three consecutive days. Results indicated that the implemented SPE and assay procedure displayed acceptable accuracy and precision. DA release from the NESD was performed over a period of 30 days (FIG. 39). The DA release from the NESD produced a peak at 3 days in both the CSF and plasma, the CSF concentration of DA being 28% while the plasma concentration was only 1.2% of the total concentration administered. The pharmacokinetic profile for plasma maintained low levels of DA release throughout the 30 days of the study whereas the CSF concentration of DA peaked at 3 days and thereafter maintained low levels of DA release for the time. Overall, the NESD was implanted at the site of action and therefore substantially improved the delivery of DA to the brain. In addition, DA concentrations in the plasma were minimal and therefore could culminate in a drastically reduced side-effects profile compared to orally administered L-dopa preparations.

3.15. Surgical Procedure and Wellbeing of the Animals after Implantation of the NBMS Device

Following the surgical procedure, the rats recovered well from anaesthesia and all animals resumed normal life for the duration of the study. However, a slight loss in weight was observed in the first week of the study in rats implanted with the placebo and MTX-loaded device. During the course of the study, no gross behavioural disorders or neurological signs were observed

### 3.16. Histological Findings on the Drug-Loaded NBMS Device

### A: Mid-Anterior Cerebral Section

In the dorsal part of the mid-anterior right cerebral hemisphere a surgical defect of the dura mater and leptomeninges measuring 2.05 mm on the dorsal aspect of the cerebrum was detected. The surgical implant measuring 1×2 mm could be identified in the cerebral cortex and penetrated up to the corpus callosum above the right lateral ventricle which was distorted by the implant. The implant revealed a homogenous mild basophilic staining in the H/E stained section and there was no inflammation present within the implant. The neuroparenchyma directly next to the implant showed mild inflammatory infiltrates with mainly macrophages (microglia) and gitter cells visible in the cerebral cortex. Few perivascular lymphocytes were present in the inflamed brain tissue. A mild spongiosis was also evident. Similar spongiosis could be demonstrated in the underlying corpus callusum. The rest of the cross section at this level showed no significant neuropathology.

### B: Mid-Posterior Cerebral Section

At this level the hippocampus was clearly visible but no diagnostic neuropathology could be demonstrated in the cerebral cortex as well as the underlying white matter of the brain. The aqueduct of Sylvius appeared normal.

### C: Mid-Cerebellar Section

The cerebellar grey matter as well as the cerebellar peduncle, white matter and fourth ventricle were morphologically normal

### D: Medulla Oblongata Section

In the section from the medulla oblongata posterior to the fourth ventricle no pathology was present in the leptomeninges and neuroparenchyma. The central canal and white matter of the medulla oblongata appeared morphologically normal.

### 3.16. Histological Findings on the Placebo NBMS Device

### A: Mid-Anterior Cerebral Section

In the dorsal aspect of the right cerebral hemisphere a defect in the dura mater measuring 2.00 mm could be demonstrated. In the underlying mid-dorsal cerebrum the surgical implant measured 1.10×2.3 mm. There were no morphological differences in the appearance of the implant when compared with similar drug-loaded implant. The surgical defect extended in the cortex up to the corpus callosum above the right lateral ventricle. Minimal inflammation was present in the brain tissue along the surgical implantation site. A few microglia and gitter cells were identified in the cerebral cortex at the junction with the implant. Minimal status spongiosis was visible.

### B: Mid-Posterior Cerebral Section

No neuropathology was present at this level of the brain:

### C: Mid-Cerebellar Section:

The cerebellar grey matter as well as the underlying cerebellar peduncle and white matter appeared morphologically normal. The section includes the fourth ventricle.

### D: Medulla Oblongata Section:

No lesions were detected in the section of the medulla oblongata posterior to the fourth ventricle.

The morphological evaluation confirmed in the dorsal parts of the mid-anterior cerebral sections from the drug-loaded as well as the placebo implants a surgical-induced defect and the implanted material. Thirty days post implantation, organization was visible where microglia were clearing the damaged tissue in both the anterior cerebral cortical sections (drug-loaded implant and placebo implant). The inflammatory reaction in the neuroparenchyma along the implant was graded mild in the drug-loaded implantation site and minimal in the placebo site. At the other levels of the cerebrum, cerebellum and medulla oblongata no neuropathology could be detected in the H/E stained sections from the drug-loaded and placebo specimens. Both the placebo device and the drug-loaded device were biocompatible with the brain tissue. Tissue inflammation was mainly induced by the surgical procedure. Thus, the composite PVA/PLA polymer provides a suitable material which can be employed successful for the development of an implant for interstitial delivery of chemotherapeutic agents.

### 4. CONCLUSIONS

The DEE of DA within the CAP nanoparticles was relatively high and compensated for the rapid in vitro release of DA from the nanoparticles. SEM and TEM images further established the uniformity and sphericity of the DA-loaded CAP nanoparticles with FTIR analysis revealing the presence of both CAP and DA within the nanoparticles. Zetasize analysis confirmed the stability of the nanoparticles within the desirable nano-size range. Significant shifts in thermal events noted with TMDSC analysis of the DA-loaded CAP nanoparticles and NESD supported the mechanism by which modulated release of DA occurred from the device. Biometric simulation and prototyping technology in conjunction with Box-Behnken statistical experimental designs as preparation and optimization strategies for the scaffold and nanoparticles proved robust in selecting optimal components for assembling the NESD. In vitro and in vivo DA release confirmed that the NESD provided higher levels and controlled delivery of DA in the CSF of the Sprague Dawley rat model and thus may serve as a desirable platform for the site-specific delivery of DA for the chronic management of PD.

The employment of a Box-Behnken experimental design for the optimization of the various polymeric scaffold and drug-loaded nanoparticle formulations proved successful in the selection of single candidate formulations intended for the proposed therapeutic applications.

The use of intricate computational models and structural rationalization techniques played a critical role in predicting the structural conformation of the synthesized biopolymeric membrane. Computational modeling has provided a mechanistic insight to further comprehend the formation, molecular structural characteristics, physicomechanical properties and the ability to entrap and modulate the release of MTX from the biopolymeric membrane. The stupendous physicomechanical properties of the membrane resulted from a superior balance of the polymeric phases employed and the addition of TEA which provided a synergistic approach in improving the biaxial extensibility, toughness of the membrane and the ability to modulate the drug release in a tri-phasic manner suitable for the novel delivery of MTX. The present biopolymeric membrane systems which can be fabricated by using various combinations of raw materials within the determined specified limits. The biopolymeric membrane systems can serve as implantable carriers for chemotherapeutic molecules like MTX and premetrex (PMT) for the treatment of primary brain tumors. Drug release can be further modulated by incorporating nanostructures within the biopolymeric membrane systems. High drug entrapment efficiencies were obtained with lower concentrations of TEA. MTX was added last during formulation, therefore as the concentration of TEA was increased the crosslinking density of the membranes increased and less drug was entrapped in the network structure. The order of addition of the components was found to be significant. MTX was added before the addition of TEA for superior drug entrapment efficiency. Drug release was depended on the concentration of PVA. Slower drug release was obtained for formulations comprising higher quantities of PVA. When PLA was consumed in the reaction, the excess stannous octoate reacted with the unreated hydroxyl groups on the PVA backbone and resulted in the formation of strong crosslinks that formed a highly dense networked structure slowing drug release. A method for preparing drug-loaded polymeric membranous scaffolds has been developed. Factors that can potentially affect drug release and the membrane erosion rate have been realized. Optimisation of the formulation will be performed in order to attain slower degradation capable of prolonged drug delivery in a rate-modulated manner. A biocompatible polymeric membrane embedded with drug encapsulated nanostructures capable of modulated drug delivery over a period extending from several hours to months.

### 5. ETHICAL APPROVAL

Ethics clearance was obtained from the Animal Ethics Committee of the University of the Witwatersrand for this study (Ethics Clearance No 2007/76/04).

### 6. REFERENCES

[1] Singh N, Pillay V, Choonara Y E 2007. Advances in the treatment of Parkinson's disease: Prog Neurobiol 81:29-44.
[2] Hall V J, Li J, Brundin P 2007. Restorative cell therapy for Parkinson's disease: A quest for the perfect cell. Seminars in Cell and Developmental Biol 18:859-869.
[3] Mann C, Aguilar E, Mengod G, Cortes R, Rodriguez-Oroz M C, Obeso J A 2008. Entacapone potentiates the long-duration response but does not normalize levodopa-induced molecular changes. Neurobiol Dis 32:340-348.
[4] Elkharraz K, Faisant N, Guse C, Siepmann F, Arica-Yegin B, Oger J M, Gust J, Goepferich A, Benoit J P and Siepmann J 2006. Paclitaxel-loaded microparticles and implants for the treatment of brain cancer: Preparation and physicochemical characterization. Int J Pharm 314:127-136.
[5] Whitney C M 2007. Medications for Parkinson's disease: Patient and family fact sheet. Neurologist 13:387-388.
[6] Samii A, Nutt J G, Ransom B R 2004. Parkinson's disease. The Lancet 363:1783-1793.
[7] Hely M A, Fung V S C, Morris J G L 2000. Treatment of Parkinson's disease. J Clin Sci 7:484-494.
[8] Papapetropoulos S and Mash D C 2005. Psychotic symptoms in Parkinson's disease: From description to etiology. Neurol 252:753-764.
[9] Uitti R J, Vingerhoets F J G, Hayward M, Cooper S and Snow B J 1997. Positron emission tomography (PET) measurements of striatel D₂ receptors in untreated Parkinson's disease patients with follow-up after 6 and 12 months treatment with Sinemet® or Sinemet® CR. Parkinson's and Related Disorders 3:43-46.
[10] Chen J J and Obering C 2005. A review of intermittent subcutaneous apomorphine injections for the rescue management of motor fluctuations associated with advanced Parkinson's disease. Clin Ther 27:1710-1724.
[11] Roos R A C, Tijssen M A J, Van der Velde E A and Breimer D D 1993. The influence of a standard meal on Sinemet CR absorption in patients with Parkinson's disease. Clin Neurol Neurosurg 95:215-219.
[12] Pahwa R and Koller W C 1998. Advances in the treatment of Parkinson's disease. Drugs Today 34:95.
[13] Bilensoy E 2007. Tumor targeted nanoparticles for cancer therapy Fur J Pharm Sci 32:810.
[14] Ahmad F J and Khan R K 2005. Nanotechnology: A revolution in the making. Pharma Rev 75:801-802.
[15] Levy R A, et al 1997. CT-generated porous hydroxyapatite orbital floor prosthesis as a prototype bioimplant. Am J Neuroradiol 18:1522-1525.
[16] Ono I et al, 1999. Treatment of large complex cranial bone defects by using hydroxyapatite ceramic implants. Plastic and Reconstructive Surg 104:339-349.
[17] Chua C K et al. 2000. Fabricating facial prosthesis models using rapid prototyping tools. Integrated Manufacturing Systems: Int J Manufact Tech Manage 11:42-53.
[18] Curodeau A, Sachs E, and Caldarise S 2000. Design and fabrication of cast orthopedic implants with freeform surface textures from 3D printed ceramic shell. J Biomed Mat Res 53:525-535.
[19] Porter N L, Pilliar R M and Grynpas M D 2001. Fabrication of porous calcium polyphosphate implants by solid freeform fabrication: A study of processing parameters and in vitro degradation characteristics. J Biomed Mat Res 56:504-515.
[20] Cheah C M et al. 2002. Characterization of microfeatures in selective laser sintered drug delivery devices. Proceedings of the Institution of Mechanical Engineers, Part H: J Engineer Med 216:369-383.
[21] Zmora S, Glicklis R, Cohen S 2002. Tailoring the pore architecture in 3-D alginate scaffolds by controlling the freezing regime during fabrication. Biomat 23:4087-4094:
[22] Piñón-Segundo E, Ganem-Quintana A, Alonso-Pérez V, Quintanar-Guerrero D 2005. Preparation and characterization of triclosan nanoparticles for periodontal treatment. Int J Pharm 294:217-232.
[23] Poletto F S, Fiel L A, Donida B, Re M I, Guterres S S, Pohlmann A R 2008. Controlling the size of poly(hydroxybutyrate-co-hydroxyvalerate) nanoparticles prepared by emulsification-diffusion technique using ethanol as surface agent. Colleids and Surfaces. A: Physicochem Engineer Aspects 324:105-112.
[24] Liu T X, Liu Z H, Ma K X, Sheri L, Zeng K Y and He C B 2003. Morphology, thermal and mechanical behavior of polyamide 6/layered-silicate nanocomposites. Comp. Sci Tech 63:331-337.
[25] Reading M 1993. Modulated differential scanning calorimetry-a new way forward in materials characterization. Trends Polym. Sci. 1:248-253.
[26] Ferrero M C, Velasco M V, Ford J L, Rajabi-Siahboomi A R, Muñoz A and Jiménez-Castellanos M R 1999. Determination of glass transition temperatures of some new methyl methacrylate copolymers using modulated temperature differential scanning calorimetry (MTDSC). Pharm Res 16:1464-1469.
[27] Sandor M, Bailey N A and Mathiowitz E 2002. Characterization of polyanhydride microsphere degradation by DSC. Polymer 43:279-288.
[28] Redhead M, Davis S S, Ilium L 2001. Drug delivery in poly(lactide-co-glycolide) nanoparticles surface modified with poloxamer 407 and poloxamine 908: In vitro characterization and in vivo evaluation. J Control Rel 70:353-363.
[29] Farokhzad O C and Robert Langer R 2006. Nanomedicine: Developing smarter therapeutic and diagnostic modalities. Adv Drug Deliv Rev 58:1456-1459.
[30] Bawa R, Nanotechnology Patents and Challenges, 2004, ipFrontline.com.
[31] Du Toit L. C, Pillay V, Choonara Y, Pillay S, Harilall S, Patenting of nanoparticles in drug delivery: No small issue, (Recent Patents on Drug Delivery and Formulations), 1, Number 2, (2007), pp. 131-142.
[32] Roney C, Kulkarni P, Arora V, Antich P, Bente F, Wu A, Mailikarjuana N. N, Manohar S, Liang H, Kulkarni A. R, Sung H, Sairam M and Aminabhavi T. M, Targeted nanoparticles for drug delivery through the blood-brain barrier for Alzheimer's disease, (Journal of Controlled Release), 108, Issue 2-3, (2005), pp. 193-214.
[33] Hood E. Nanotechnology: Looking as we leap, (Environmental Health Perspective), 112, Issue 13, (2004), pp. 740-749.
[34] Park K, Nanotcchnology: What it can do for drug delivery, (Journal of Controlled Release), 170, issue 1-2, (2007), pp. 1-3.
[35] Park J. H, Lee S, Kim J, Park K, Kim K and Kwon I. C, Polymeric nanomedicine for cancer chemotherapy, (Progress in Polymer Science), (2007).
[36] Feijen J, Grijpma D. W and Zhang Z, Poly(trimethylene carbonate) and monomethoxy poly(ethylene glycol)-block-poly(trimethylene carbonate) nanoparticles for the controlled release of dexamethasone, (Journal of Controlled Release), (2005).
[37] Pison U, Welte T, Giersig M and Groneberg D. A, Nanomedicine for respiratory disease, (European Journal of Pharmacology), 533, Issues 1-3, (2006), pp. 341-350.
[38] Popovic N and Brundin P, Therapeutic potential of controlled drug delivery systems in neurodegenerative diseases, (International Journal of Pharmaceutics), (2005).
[39] Reis C. P, Neufeld R. J, Ribeiro A. J and Veiga F, Nanoencapsulation II. Biomedical applications and current status of peptide and protein nanoparticulate delivery systems, (Nanomedicine: Nanotechnology, Biology and Medicine), 2, issue 2, (2006), pp. 53-65.
[40] Liu L. S, Fishman M. L, Hicks K. B and Kende M, Interaction of various pectin formulations with porcine colonic tissues, (Biomaterials), 26, Issue 29, (2005), pp. 5907-5916.
[41] Rezwan K, Chen Q. Z, Blaker J. J and Boccaccini A. R, Biodegradable and bioactive porous polymer/inorganic composite scaffolds for bone tissue engineering, (Biomaterials), 27, Issue 18, (2006), pp. 3413-3431.
[42] Luong-Van E, Grondahl L, Ngiap Chua K, Leong K. W, Nurcombe V and Cool S. M, Controlled release of heparin from poly(ε-caprolactone) electrospun fibers, (Biomaterials), 27, Issue 9, (2006), pp. 2042-2050.
[43] McKenzie J. L, Waid M. C, Shi R and Webster T. J, Decreased functions of astrocytes on carbon nanofiber materials, (Biomaterials), 25, Issue 7-8, (2003), pp. 1309-1317.
[44] Bajpai S. K and Sharma S, Investigation of swelling/degradation behaviour of alginate beads crosslinked with Ca2+ and Ba2+ ions, (Reactive and Functional Polymers), 59, Issue 2, (2004), pp. 129-140.
[45] Zactiti E. M and Kieckbusch T. G, Potassium sorbate permeability in biodegradable alginate films: Effects of the antimicrobial agent concentration and crosslinking degree, (Journal of Food Engineering), 77, Issue 3, (2006), pp. 46-467.
[46] Rokhade A. P, Agnihotri S. A, Patil S. A, Mallikarjuna N. N, Kulkarni P. V and Aminabhavi T. M, Semi-interpenetrating polymer network microspheres of gelatin and sodium carboxymethyl cellulose for controlled release of ketorolac tromethamine, (Carbohydrate Polymers), 65, Issue 3, (2006), pp. 243-252.
[47] Zweers M. L. T, Engbers G. H. M, Grijpma D. W and Feijen J, In vitro degradation of nanoparticles prepared from polymers based on DL-lactide, glycolide and polyethylene oxide), (Journal of Controlled Release), 100, Issue 3, (2004), pp. 347-356.
[48] Karp M and Langer R 2007 Development and therapeutic application of advanced biomaterials Curr. Opinion Biotech. 18 454-459
[49] Popovic N and Brundin P 2006 Therapeutic potential of controlled drug delivery systems in neurodegenerative diseases Int. J. Pharm. 34 120-126
[50] Ponce S, Orive G, Gascon A R, Hernandez R M and Pedraz J L 2005 Nanoparticles prepared with different biomaterials to immobilize GDNF secreting fibroblasts Int. J. Pharm. 293 1-10
[51] Whittlerey K J and Shea C D 2004 Delivery systems for small molecule drugs, proteins, and DNA: the neuroscience/biomaterials interface Exp. Neuro. 190 1-16
[52] Ramehandan M and Robinson D 1998 In vitro and in vivo release of ciprofloxacin from PLGA 50:50 implants J. Control. Rel. 45 167-175
[53] Joachim Kohn, William J Wels and Doyle Knight 2007 A new approach to the rationale discovery of biomaterials Biomat. 28 4171-4177
[54] Eisenberg P 1995 Executive summary of NIH workshop on biomaterials and medical implants 16-17 October, Bethesda Md.: National Institutes of Health.
[55] Eksterowics J E, Evensen E, Lemmen C, Brady G P, Lanctot J K, Bradley E K, et al 2002 Coupling structure-based design with combinatorial chemistry: application of active site derived pharmacophores with informative library design J. Mol. Graph. Model 20 469-77
[56] Stanton R V, Mount J and Miller J L 2000 Combinatorial library design: maximising model-fitting compounds within matrix synthesis constraints J. Chem. Inf. Comput. Sci. 40 701-705
[57] Kirkpatrick D L, Watson S and Ulhaq S 1999 Structure-based drug design: combinatorial chemistry and molecular modeling Comb. Chem. High Throughput Screen. 2 211-221
[58] Ironi L and Tentoni S 2003 A model based approach to the assessment of physicochemical properties of drug delivery materials Comput. Chem. Eng. 27 803-812
[59] Brocchini S, James K, Tangpasuthadol V and Khon J 1997 A combinatorial approach for polymer design J. Am. Chem. Soc. 1194553-4554
[60] Selvam P, Tsuboi H, Kubo M, Koyaba M and Miyamoto A 2005 Tight-binding quantum chemical molecular dynamics method: a novel approach to the understanding and design of new materials and catalysts Catalysis Today 100 11-25
[61] Tuzun R E, Noid D W and Sumpter B G 1998 Computer simulation of complex strongly coupled nanometer-scale systems: breaking the billion atom barrier Comput. Math. Applic. 35 93-100
[62] Smith R J, Agnieszka S, Weber N, Knight D, Abramson S and Khon J 2004 Integration of combinatorial synthesis, rapid screening, and computational modeling in biomaterials development Macromol. Rapid Commun. 25 27-40
[63] Gao H 2001 Modeling strategies for nano- and biomaterials In: Ruhle M, Dosch H, Mittemeijer E J, Van de Voorde, Editors, European White Book on Fundamental Research in Material Science 144-148
[64] Abramson S D, Alexe G, Hammer P L and Kohn J A 2005 Computational approach to predicting cell growth on polymeric biomaterials J. Biomed. Mat. Res. 73 116-124
[65] Gubskaya A V, Kholodovych V, Knight D, Kohn J and Welsh WJ 2007 Prediction of fibrinogen adsorption for biodegradable polymers: Integration of molecular dynamics and surrogate modeling Polym. 48 5788-5801
[66] Duce C, Micheli A, Solaro R, Starita A and Tine M R 2006 Prediction of chemical-physical properties by neural networks for structures Macromol. Symp. 234 13-19
[67] Holder AJ and Kilway KV 2005 Rational design of dental materials using computational chemistry Dental Mat. 21 47-55
[68] Omathanu Pillai, Anand Babu Dhanikula and Ramesh Pachagnula 2001 Drug delivery an odyssey of 100 years Curr. Opin. Chem. Biol. 5 439-446
[69] Esperon S, Bossy-Nobs L, Petropolous L K, Gurny R and Guex-Crosier YA 2008 Biodegradable drug delivery system for the treatment of postoperative inflammation Int. J. Pharm. 352 240-247
[70] Jose L, Arias M, Aldofina R, Margarita L and Delgado A V 2008 Poly(alkylcyanoacrylate) colloidal particles as vehicles for antitumor drug delivery: a comparative study Colloids and Surfaces B: Biointerfaces 62 64-70
[71] Dorothee R, Andreas L, Ilka P, Marsha A M and Ralf-Peter F 2006 Biocompatibility testing of novel multifunctional biomaterials for tissue engineering applications in head and neck surgery: an overview Eur. Arch. Otorhinolaryngol. 263 215-222
[72] Foernkier E, Passirani C, Montero-Menei C N and Benoit J P 2003 Biocompatibility of implantable synthetic polymeric drug carriers: focus on brain biocompatibility Biomat. 24 3311-3331
[73] Rajeev A J 2000 The manufacturing techniques of various drug loaded biodegradable poly(lactide-co-glycolide) (PLGA) devices Biomat. 21 2475-2490
[74] Anderson J M and Shive M S1997 Biodegradation and biocompatibility of PLA and PLGA microspheres Adv. Drug Deliv. Rev. 28 5-24
[75] Singh M, Briones M, Ott G and O Hagon D 1997 Cationic microparticles: a potent delivery system for DNA vaccines Proc. Natl. Acad. Sci. USA 811-816
[76] Cui C and Schwendenman SP 2001 Surface entrapment of polylysine in biodegradable poly(DL-lactide-co-glycolide) microparticles Macromol. 34 8426-8433
[77] Burrem D A, Zylsta E, Lonsburg P T and Langer R 1993 Synthesis and RDG peptide modification of a new biodegradable copolymer poly(lactic acid)-co-lysine J. Am. Chem. Soc. 115 11011-11011
[78] Kissel T, Breitenbach A and Pistel K F 2000 Biodegradable comb polyesters. Part II. Erosion and release properties of poly(vinyl alcohol)-g-poly(lactic-co-glycolic acid) Polym. 41 4781-4792
[79] Kissel T, Lea A D and Wittmar M 2005 The role of branched polyesters and their modifications in the development of modern drug delivery vehicles J. Control. Rel. 101 137-149
[80] Mohile N A and Abrey L E 2007 Primary central nervous system lymphoma Neurol. Clin. 25 1193-1207.
[81] Dufrene Y F and Lee G U 2000 Advances in the characterization of supported lipid films with the atomic force microscope Biochimica et Biophysica Acta (BBA)-Biomembranes 1509 14-41
[82] Rinia H A, Wurpel G W H and Muller M 2006 Chapter 4 visualization and characterization of domains in supported model membranes Adv. Planar Lipid Bilayers and Liposomes 3 85-123
[83] Rahimpour A, Madaeni S S and Mehdipour-Ataei S 2008 Synthesis of a novel poly(amide-imide) (PAI) and preparation and characterization of PAI blended polyethersulfone (PES) membranes J. Membr. Sci. 311 349-359
[84] Zeni M, Riveros R, de Souza J F, Mello K, Meireles C and Filho G R 2008 Morphologic analysis of porous polyamide 6,6 membranes prepared by phase inversion Desalination 221 294-297
[85] Esperon S, Bossy-Nobs L, Petropoulos I K, Gurny R, Guex-Crosier Y, A biodegradable drug delivery system for the treatment of postoperative inflammation. Int. J. Pharm. 2008; 352:240-247.
[86] Brem H, Gabikian P, Biodegradable polymer implants to treat brain tumors. J. Cont. Rel. 2001; 74:63-67.
[87] Whittlesey K J, Shea L D, Delivery systems for small molecule drugs, proteins and DNA: the neuroscience/biomaterial interface. Exp. Neur. 2004; 190:1-16.
[88] Karp J M, Langer R, Development and therapeutic applications of advanced biomaterials. Cur. Opi. Biot. 2007; 18:454-459.
[89] Nair L S, Laurencin C T, Biodegradable polymers as biomaterials. Prog. Polym. Sci. 2007; 32:762-798.
[90] Fournier E, Passirani C, Montero-Menei C N, Benoit J P, Biocompatibility of implantable synthetic polymeric carriers: focus on brain biocompatibility. Biom. 2003; 24:3311-3331.

1. A polymeric pharmaceutical dosage form for the delivery, in use, of at least one pharmaceutical composition in a rate-modulated and site-specific manner, said dosage form comprising a biodegradable, crosslinked, polymeric, scaffold incorporating nanoparticles or microparticles loaded with at least one active pharmaceutical ingredient (API) which, in use, are released from said scaffold as the polymer or polymers degrade in a human or animal body, the polymer or polymers being selected to degrade in response to or in the absence of specific biological stimuli and, thus, release the API or APIs in an area where said stimuli are encountered, characterised in that the scaffold serves as a substrate for said nanoparticles or microparticles loaded with one or more APIs which is or are used to treat a neurological condition and which degrades such that, in use, the API or APIs is or are able to circumvent the blood-brain barrier.

2. (canceled)

3. A polymeric pharmaceutical dosage form as claimed in claim 1 in which the or each polymer making up the polymeric scaffold is hydrophilic.

4. A polymeric pharmaceutical dosage form as claimed in claim 1 in which the or each polymer making up the polymeric scaffold is hydrophobic.

5. A polymeric pharmaceutical dosage form as claimed in claim 1 in which the or each polymer making up the polymeric scaffold are a combination of hydrophilic and hydrophobic polymers selected from the group consisting of polycaprolactone (PCL), pectins, and alginates as native polymers.

6. A polymeric pharmaceutical dosage form as claimed in claim 5 in which the polymeric scaffold is formed from poly (D1L-lactide) (PLA) and polymethacrylate (Eudragit S100/ES100) polymers.

7. A polymeric pharmaceutical dosage form as claimed in claim 1 in which at least one of the or each polymer making up the polymeric scaffold includes a modifier chemical which causes the or each polymer to undergo, in use, a controlled swelling, shrinking and/or erosion.

8. A polymeric pharmaceutical dosage form as claimed in claim 7 in which the modifier is selected from a group of substances that interact with the or each polymer to reduce the swellibility of the latter.

9. A polymeric pharmaceutical dosage form as claimed claim 1 characterized in that the inherent polymeric structure of the native polymer or polymers is manipulated through crosslinking using crosslinking reagents comprising biocompatible inorganic salts that are ionic of a mono-, di-, or trivalent nature.

10. (canceled)

11. A polymeric pharmaceutical dosage form as claimed in claim 9 in which the biocompatible inorganic salts are selected from the group consisting sodium chloride, aluminium chloride or calcium chloride.

12. (canceled)

13. (canceled)

14. (canceled)

15. A polymeric pharmaceutical dosage form as claimed in claim 1 characterized in that the dosage form is surgically implantable in use.

16. A polymeric pharmaceutical dosage form as claimed in claim 1 in which the dosage form is insertable, in use, into a body cavity selected from the group consisting of a nasal passage, rectum or vagina.

17. A polymeric pharmaceutical dosage form as claimed in claim 1 in which the dosage form comprises a barium-alginate scaffold incorporating CAP dopamine-loaded nanoparticles and is adapted to treat, in use, Parkinson's disease.

18. A polymeric pharmaceutical dosage form as claimed in claim 1 in which the dosage form comprises a membranous-like polymeric scaffold incorporating API-loaded nanoparticles and is adapted to treat, in use, brain tumors.

19. A polymeric pharmaceutical dosage form as claimed in claim 1 in which the dosage form comprises a polymeric scaffold incorporating API-loaded nanoparticles and is adapted to treat, in use, Aids Dementia Complex.

20. A method of preparing a polymeric pharmaceutical dosage form for the delivery, in use, of at least one pharmaceutical composition in a rate-modulated and site-specific manner, said method comprising preparing a biodegradable, polymeric, scaffold, loading nanoparticles or microparticles with at least one active pharmaceutical ingredient (API) and incorporating the nanoparticles or microparticles into the scaffold so that the nanoparticles or microparticles, and, consequently, the API is released, in use, from said scaffold as the polymer or polymers degrade in a human or animal body, the polymer or polymers being selected to degrade in response to or in the absence of specific biological stimuli and, thus, release the API or APIs in an area where said stimuli are encountered characterized in that the said polymeric scaffold is manufactured from a combination of hydrophilic and hydrophobic polymers.

21. A method of preparing a polymeric pharmaceutical dosage form as claimed in claim 20 in which the polymeric scaffold is a membranous-like polymeric scaffold which, in use, releases the or each API in a desired rate modulated manner which is achievable by selecting one or more polymers making up the scaffold according to the rate of biological degradation of said polymers and the consequent release of the or each API in the human or animal body.

22. (canceled)

23. (canceled)

24. A method of preparing a polymeric pharmaceutical dosage form as claimed in claim 20 wherein the hydrophilic and hydrophobic polymers are selected from the group consisting of polycaprolactone (PCL), pectins, and alginates as native polymers, to form the polymeric scaffold.

25. A method of preparing a polymeric pharmaceutical dosage form as claimed in claim 24 in which includes forming the polymeric scaffold from poly (D1L-lactide) (PLA) and polymethacrylate (Eudragit S100/ES100) polymers.

26. A method of preparing a polymeric pharmaceutical dosage form as claimed in claim 20 which includes manipulating through crosslinking using crosslinking reagents of biocompatible inorganic salts that are ionic of a mono-, di-, or, trivalent nature selected from the group consisting sodium chloride, aluminium chloride or calcium chloride, the inherent polymeric structure of the native polymer or polymers.

27. A method of preparing a polymeric pharmaceutical dosage form as claimed in claim 26 which includes combining any one of > a number of combination permutations of hydrophilic and hydrophobic polymeric selected from PCL, matrices, active pharmaceutical compositions and inorganic salt(s), and wherein the release profile of the pharmaceutical composition or compositions are governed by the crosslinking reagent, polymer matrix size and hydration, porosity, embedded nanostructures and the architectural structure of the resulting polymeric network as well as the degree of hydration of the polymer or polymers which, in turn, depends on the pKa, concentration and valence of release rate-modulating chemical substances used, to provide for a desired, rate-modulatable, release of the or each API.

28. (canceled)

29. (canceled)

30. (canceled)
